(19) United States
(12) Patent Application Publication
(10) Pub. No.: US 2014/0121403 A1
(43) Pub. Date: May 1, 2014

(54) Integrated Process for the Production of Acrylic Acids and Acrylates

(71) Applicant: Celanese International Corporation, Irving, TX (US)

(72) Inventors: Dick Nagaki, The Woodlands, TX (US); Craig J. Peterson, Houston, TX (US); Elizabeth Bowden, Houston, TX (US); Josefina T. Chapman, Houston, TX (US); Sean Mueller, Pasadena, TX (US)

(21) Appl. No.: 13/928,850

(22) Filed: Jun. 27, 2013

Related U.S. Application Data
(63) Continuation-in-part of application No. 13/792,814, filed on Mar. 11, 2013.
Application No. 13/792,814 is a continuation-in-part of application No. 13/664,494, filed on Oct. 31, 2012.
Application No. 13/664,494 is a continuation-in-part of application No. 13/664,477, filed on Oct. 31, 2012, now Pat. No. 8,669,201.
Application No. 13/664,477 is a continuation-in-part of application No. 13/664,478, filed on Oct. 31, 2012.

(51) Int. Cl. C07C 51/377; C07C 67/08

(52) U.S. Cl. CPC C07C 51/377; C07C 67/08
USPC 560/210; 562/599

(57) ABSTRACT

A process for producing an acrylate product from methanol and acetic acid, in which, in a reaction zone A, the methanol is partially oxidized to formaldehyde in a catalyzed gas phase reaction, the product gas mixture A obtained and an acetic acid source are combined to form a reaction gas input mixture B which comprises acetic acid in excess over formaldehyde, and the formaldehyde in reaction gas input mixture B is aldol-condensed to acrylic acid in the presence of a catalyst in a reaction zone B to form an acrylic acid-containing product gas mixture B from which an acrylate product stream may be separated. Suitable aldol condensation catalysts include vanadium-bismuth, vanadium-titanium-bismuth, vanadium-bismuth-tungsten, vanadium-titanium-tungsten, vanadium-titanium and vanadium-tungsten.

### CROSS REFERENCE TO RELATED APPLICATIONS

This application is a continuation-in-part of U.S. patent application Ser. No. 13/792,814, which was filed on Mar. 11, 2013; U.S. patent application Ser. No. 13/664,494, which was filed on Oct. 31, 2012; U.S. patent application Ser. No. 13/664,477, which was filed on Oct. 31, 2012; and U.S. patent application Ser. No. 13/664,478, which was filed on Oct. 31, 2012. The entireties of these applications are incorporated by reference herein.

### FIELD OF THE INVENTION

The present invention relates generally to the production of acrylic acid via a process that integrates a methanol oxidation reaction zone, an aldol condensation reaction zone, and a separation zone.

### BACKGROUND OF THE INVENTION

The present invention relates to a process for preparing acrylic acid from methanol and acetic acid. The present invention also relates to the preparation of conversion products from acrylic acid thus obtained.

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes: (1) the reaction of acetylene with water and carbon monoxide; and/or (2) the reaction of an alcohol and carbon monoxide, in the presence of an acid, e.g., hydrochloric acid, and nickel tetracarbonyl, to yield a crude product comprising the acrylate ester as well as hydrogen and nickel chloride. Another conventional process involves the reaction of ketene (often obtained by the pyrolysis of acetone or acetic acid) with formaldehyde, which yields a crude product comprising acrylic acid and either water (when acetic acid is used as a pyrolysis reactant) or methane (when acetone is used as a pyrolysis reactant). These processes have become obsolete for economic, environmental, or other reasons.

More recent acrylic acid production processes have relied on the gas phase oxidation of propylene, via acrolein, to form acrylic acid. The reaction can be carried out in single- or two-step processes but the latter is favored because of higher yields (see, for example, DE-A 103 36 386). The oxidation of propylene produces acrolein, acrylic acid, acetaldehyde and carbon oxides. Acrylic acid from the primary oxidation can be recovered while the acrolein is fed to a second step to yield the crude acrylic acid product, which comprises acrylic acid, water, small amounts of acetic acid, as well as impurities such as furfural, acrolein, and propionic acid. Purification of the crude product may be carried out by azeotropic distillation. Although this process may show some improvement over earlier processes, this process suffers from production and/or separation inefficiencies. In addition, this oxidation reaction is highly exothermic and, as such, creates an explosion risk. As a result, more expensive reactor design and metallurgy are required.

Propylene can be produced from mineral oil with comparatively low production costs. In view of the foreseeable shortage in the fossil resource of mineral oil, however, there may be a need for processes for preparing acrylic acid from other raw materials.

WO 2005/093010 proposes the use of the two-stage heterogeneously catalyzed partial gas phase oxidation of propylene to acrylic acid. The propylene may be obtained from methanol. The advantage of such a procedure is that methanol is obtainable both from base fossil raw materials such as coal, for example brown coal and hard coal as disclosed in WO 2010/072424, and/or natural gas, as disclosed in WO 2010/067945. Both of these sources have a much longer lifetime than mineral oil. A disadvantage of the procedure proposed in WO 2005/093010, however, is that the selectivity to propylene based on methanol converted is less than 70 mol %, which is unsatisfactory (in addition to propylene, for example, ethylene and butylene are also formed).

WO 2008/023040, for example, has disclosed the preparation of acrylic acid and the conversion products thereof starting from glycerol, a renewable raw material. A disadvantage of such a procedure, however, is that glycerol is only feasibly obtainable as a renewable raw material essentially as a coproduct of biodiesel production. And the current energy balance of biodiesel production is unsatisfactory.

Some references, for example, DE-A 102006024901, have disclosed the preparation of acrylic acid from propane, which is a raw constituent of natural gas. A disadvantage of such a method, however, is that propane is generally high unreactive.

The aldol condensation reaction of formaldehyde and acetic acid and/or carboxylic acid esters has been disclosed in literature. This reaction forms acrylic acid and is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987).

US Patent Publication No. 2012/0071688 discloses a process for preparing acrylic acid from methanol and acetic acid in which the methanol is partially oxidized to formaldehyde in a heterogeneously catalyzed gas phase reaction. The product gas mixture thus obtained and an acetic acid source are used to obtain a reaction gas input mixture that comprises acetic acid and formaldehyde. The acetic acid is used in excess over the formaldehyde. The formaldehyde present in reaction gas input mixture is aldol-condensed with the acetic acid via heterogeneous catalysis to form acrylic acid. Unconverted acetic acid still present alongside the acrylic acid in the product gas mixture is removed therefrom and is recycled to the reaction gas input mixture.

Although the methanol oxidation reaction and the aldol condensation reaction are disclosed in US Patent Publication No. 2012/0071688, the aldol condensation catalysts disclosed therein can be improved upon in terms of providing high acetic acid conversions and acrylate production yields.

Thus, the need exists for a process for producing purified acrylate product, e.g., acrylic acid, which utilizes improved aldol condensation catalysts capable of providing high acetic acid conversions and acrylate production yields.

The references mentioned above are hereby incorporated by reference.

### SUMMARY OF THE INVENTION

In one embodiment, the invention relates to a process for producing an acrylate product. The process comprises the step of reacting a reaction gas mixture A comprising methanol, oxygen, and at least one diluent gas other than steam to form a product gas mixture A. The product gas mixture A may comprise formaldehyde, steam, and at least one inert diluent gas other than steam. The reaction may be conducted in a first reaction zone. The process may further comprise the step of combining at least a portion of the product gas mixture A and acetic acid to form a reaction gas mixture B in the presence of at least one aldol condensation catalyst. The reaction gas mixture B may comprise acetic acid, formaldehyde, steam, and at least one diluent gas other than steam. The process may further comprise the step of reacting at least a portion of the acetic acid in the reaction gas input mixture B with at least a portion of the formaldehyde in the reaction gas input mixture B to form a product gas mixture B. The product gas mixture B may comprise acrylic acid, acetic acid, steam, and at least one inert diluent gas other than steam. The reaction may be conducted in a second reaction zone. Aldol condensation catalysts which have been found to be particularly useful are catalysts comprising an active phase comprising vanadium, titanium, bismuth, and/or tungsten. These catalysts may comprise from 0.3 wt. % to 32 wt. % vanadium; and/or from 0.1 wt. % to 75 wt. % bismuth and the molar ratio of vanadium to bismuth in the active phase of the at least one aldol condensation catalyst composition may be at least 0.02:1. The aldol condensation catalyst may correspond to the formulae VₐBi_{b}P_{c}O_{d}; VₐBi_{b}Ti_{c}P_{d}Oₑ; VₐBi_{b}W_{c}P_{d}Oₑ; VₐTi_{b}W_{c}P_{d}Oₑ; and/or VₐTi_{b}P_{c}O_{d}(oxide additive)ₑ. In one embodiment, the active phase may further comprise titanium, e.g., from 0.015 wt. % to 22 wt. % titanium. In one embodiment, the active phase may further comprise tungsten, e.g., from 0.1 wt. % to 61 wt. % tungsten. A molar ratio of vanadium to tungsten in the active phase of the catalyst composition may be at least 0.033:1, and a molar ratio of bismuth to tungsten in the active phase of the catalyst composition may be at least 0.0033:1. A molar ratio of vanadium to bismuth in the active phase of the catalyst composition may be at least 0.033:1, and a molar ratio of vanadium to tungsten in the active phase of the catalyst composition may be at least 0.033:1. The at least one oxidation catalyst comprises a catalytically active material which is a mixed oxide of the general formula [Fe₂(MoO₄)₃]₁[M¹ₘOₙ]_{q}. The aldol condensation catalyst may comprise an active phase comprising vanadium, titanium and tungsten, and a molar ratio of vanadium to tungsten in the active phase of the catalyst composition may be at least 0.02:1. The active phase may comprise from 0.2 wt. % to 30 wt. % vanadium; and/or from 0.016 wt. % to 20 wt. % titanium; and/or from 0.11 wt. % to 65 wt. % tungsten. The aldol condensation catalyst may comprise an active phase comprising vanadium and titanium, and a molar ratio of vanadium to titanium in an active phase of the catalyst composition is greater than 0.5:1. The aldol condensation catalyst may further comprise at least one oxide additive in an amount of at least 0.1 wt % based on the total weight of the aldol condensation catalyst; and the molar ratio of oxide additive to titanium in an active phase of the at least one aldol condensation catalyst may be at least 0.05:1.

### DETAILED DESCRIPTION OF THE INVENTION

Production of unsaturated carboxylic acids such as acrylic acid and methacrylic acid and the ester derivatives thereof via most conventional processes have been limited by economic and environmental constraints. In the interest of finding a new reaction path, the aldol condensation reaction of acetic acid and formaldehyde has been investigated. The formaldehyde may be formed via the oxidation of methanol. This aldol condensation reaction may yield a unique crude product that comprises, inter alia, a higher amount of (residual) formaldehyde, which is generally known to add unpredictability and problems to separation schemes. Although the aldol condensation reaction of acetic acid and formaldehyde is known, there has been little if any disclosure relating to aldol condensation catalysts that may be employed to effectively provide purified acrylic acid from the aldol condensation crude product.

One benefit demonstrated by the embodiments of the present invention is that the acetic acid is itself obtainable in a simple and industrially tried and tested manner proceeding from methanol, by carbonylation thereof with carbon monoxide (see, for example, Industrielle Organische Chemie [Industrial Organic Chemistry], Klaus Weissermel and Hans-Jurgen Arpe, Wiley-VCH, Weinheim, 5th edition (1998), p. 194 to 198).

In this document, base fossil raw materials shall be understood to mean base raw materials which, like brown coal, hard coal, natural gas and mineral oil, for example, are formed from degradation products of dead plants and dead animals.

In contrast, in this document, renewable raw materials shall be understood to mean those raw materials which are obtained from fresh biomass, e.g., from (new) vegetable and animal material which is being newly grown (in the present) and will be grown in the future.

One advantage of an acrylic acid preparation process based on the raw material methanol is that the methanol can be obtained via synthesis gas (gas mixtures of carbon monoxide and molecular hydrogen) in principle from all carbonaceous base fossil materials and all carbonaceous renewable raw materials. As in the case of methane, the molecular hydrogen required may already be present in the carbon carrier (a process for obtaining methane from biogas or biomass is described, for example, in DE-A 102008060310 and EP-A 2220004). An alternative hydrogen source is water, from which molecular hydrogen can be obtained, for example, by means of electrolysis. The oxygen source is generally air (see, for example, WO 10-060236 and WO 10-060279). A suitable renewable carbonaceous raw material for synthesis gas production is, for example, lignocellulose (see, for example, WO 10-062936). It is also possible to obtain synthesis gas by coupling the pyrolysis of biomass directly with steam reforming.

The present invention thus provides a process for preparing acrylic acid from methanol and acetic acid, which comprises the following steps. A stream of a reaction gas input mixture A comprising the methanol and molecular oxygen reactants and at least one inert diluent gas other than steam is directed through a first reaction zone A, which is charged with at least one oxidation catalyst A. The reaction gas input mixture may comprise oxygen and methanol, preferably in a molar ratio of at least 1, e.g., at least 2, at least 5, or at least 10. In the course of passage through reaction zone A, methanol present in the reaction gas input mixture A is oxidized under heterogeneous catalysis to form formaldehyde and steam, which exit as product gas mixture A. Product gas mixture A comprises formaldehyde, steam, and at least one inert diluent gas other than steam. The oxidation reaction may, in some embodiments, be conducted with or without excess molecular oxygen. Product gas mixture A leaves reaction zone A. In one embodiment, molecular oxygen and/or further inert diluent gas other than steam are supplied to the reaction gas mixture A flowing through reaction zone A. Product gas mixture A may, in some embodiments, comprise methanol, e.g., unconverted methanol. Optionally, the stream of product gas mixture A leaving reaction zone A may be fed to a separation zone T* and any unconverted methanol still present in product gas mixture A in separation zone T* may be removed from product gas mixture A to leave a formaldehyde-comprising product gas mixture A*. A stream of product gas mixture A* leaves reaction zone A. The process may form a stream of a reaction gas input mixture B from the product gas mixture A. The reaction gas input mixture B may comprise acetic acid, steam, at least one inert diluent gas other than steam, and formaldehyde, with or without molecular oxygen. In one embodiment, the molar amount of acetic acid, n_{HAc}, present in the reaction gas input mixture B is greater than the molar amount of formaldehyde, n_{Fd}, present in the reaction gas input mixture B. The reaction gas input mixture B may be formed by combining an acetic acid stream and at least a portion of product gas mixture A.

The reaction gas input mixture B is passed through a second reaction zone B, which is charged with at least one aldol condensation catalyst B. Formaldehyde present in reaction gas input mixture B, as it flows through reaction zone B, is condensed with acetic acid present in reaction gas input mixture B (preferably under heterogeneous catalysis) to form product gas mixture B comprising acrylic acid and water. In one embodiment, the reaction gas mixture B comprises acetic acid and formaldehyde in a molar ratio ranging from 1 to 10, e.g., from 1 to 8 or from 1 to 5. Product gas mixture B comprises acrylic acid, acetic acid, steam and at least one inert diluent gas other than steam, optionally with or without molecular oxygen. The product gas mixture B leaves reaction zone B. In one embodiment, it optionally is possible to supply further molecular oxygen and/or further inert diluent gas to the reaction gas mixture B. The stream of product gas mixture B leaving reaction zone B is fed to a separation zone T and separated in separation zone T into at least three streams X, Y and Z. The acrylic acid flow present in stream X is greater than the acrylic acid flow present in streams Y and Z together. The acetic acid flow present in stream Y is greater than the acetic acid flow present in streams X and Z together. The flow of inert diluent gas other than steam present in stream Z is greater than the flow of inert diluent gas other than steam present in streams X and Y together. Stream Y may be recycled into reaction zone B and used to obtain reaction gas input mixture B. The process of the present invention employs specific catalyst compositions as the at least one aldol condensation catalyst B. The use of these specific catalyst compositions, surprisingly and unexpectedly, provides for significant improvements in, inter alia, high acetic acid conversions and acrylate production yields as compared to processes that employ conventional catalysts. These specific catalyst compositions are discussed in detail below.

Another significant advantage of the inventive procedure is that the formaldehyde present in product gas mixture A need not be removed from product gas mixture A in order to be able to use it to obtain reaction gas input mixture B.

Instead, the formaldehyde-comprising stream of product gas mixture A leaving reaction zone A can be used as such, e.g., without conducting a removal process thereon beforehand, in order to obtain the reaction gas input mixture B. In general, for this purpose, the product gas mixture A will first be cooled (quenched) when it leaves reaction zone A in order to reduce unwanted further reactions in product gas mixture A before the introduction thereof into reaction gas input mixture B. Typically, it will be cooled as rapidly as possible to temperatures of 150 to 350° C., or 200 to 250° C.

Optionally, it is also possible to first remove a portion or the entirety of any methanol which has not been converted in reaction zone A and is still present in product gas mixture A from the latter in a separation zone T*, and then to use the remaining formaldehyde-comprising product gas mixture A* (which may pass through the liquid state in the course of the removal) to obtain reaction gas input mixture B. Advantageously, in some embodiments, the removal will be undertaken by rectificative means, e.g., a rectification column. For this purpose, product gas mixture A, optionally after preceding direct or indirect cooling, can be fed in gaseous form to the corresponding rectification column provided with cooling circuits. It is of course possible, however, first to convert those constituents whose boiling point at standard pressure (10⁵ Pa) is less than or equal to the boiling point of formaldehyde from product gas mixture A to the liquid phase (for example by condensation), and to undertake the rectification from the liquid phase. In general, such a methanol removal may also be accompanied by a removal of steam present in product gas mixture A. For the purpose of the aforementioned direct cooling, it is possible to use, for example, a liquid phase which has been withdrawn from the bottom region of the rectification column and has optionally additionally been cooled by indirect heat exchange, which is sprayed by means of appropriate nozzles into fine droplets which provide the large heat exchange area required for the hot product gas mixture A. Appropriately, in accordance with the invention, the methanol removed may be recycled into reaction zone A and used to obtain the reaction gas input mixture A. Removal of methanol from product gas mixture A, prior to its use in forming reaction gas input mixture B, is generally utilized when reaction zone A is configured such that the resulting conversion of methanol in reaction zone A, based on the single pass of product gas mixture A through reaction zone A, is not more than 90 mol %. It will be appreciated that such a methanol removal, however, can also be employed in the case of corresponding methanol conversions of not more than 95 mol %. For example, such a methanol removal can be undertaken as described in Ullmann's Encyclopedia of Industrial Chemistry, vol. A11, 5th ed., VCH Weinheim.

The oxidation catalysts A particularly suitable for charging of reaction zone A can be divided essentially into two groups.

The first of the two groups comprises silver catalysts, which have, as the active material, elemental silver whose purity is preferably ≧99.7% by weight, advantageously ≧99.8% by weight, preferably ≧99.9% by weight and most preferably ≧99.99% by weight. The corresponding processes for heterogeneously catalyzed partial gas phase oxidation of methanol to formaldehyde over these “silver catalysts” are described as silver processes (see, for example, “A. Nagy, G. Mestl: High temperature partial oxidation reactions over silver catalysts, Appl. Catal. 188 (1999), p. 337 to 353”, “H. Schubert, U. Tegtmayr, R. Schlogl: On the mechanism of the selective oxidation of methanol over elemental silver, Catalyst Letters, 28 (1994), p. 383 to 395”, “L. Lefferts, Factors controlling the selectivity of silver catalysts for methanol oxidation, thesis, University of Twente (1987)” and DE-A 2334981).

Silver oxidation catalysts A advantageous in accordance with the invention for charging of reaction zone A are disclosed, for example, in Ullmann's Encyclopedia of Industrial Chemistry, vol. A11, 5th ed., VCH, Weinheim, or in Encyclopedia of Chemical Technology, vol. 11, 4th ed., Wiley & Sons, New York, p. 929 to 949, in DE-B 1231229, in DE-B 1294360, in DE-A 1903197 and in BE patent 683130. Typically, these comprise crystals (the shape of which may also be round) of elemental silver (preferably of the abovementioned purity) which have been deposited by electrolysis of aqueous silver salt solutions and which can be poured as a fixed catalyst bed onto a perforated base (for example a perforated plate, a sieve or a mesh network (preferably likewise manufactured from silver)) (typical bed heights are 10 to 50 mm, frequently 15 to 30 mm). The total content of metals present in elemental form other than silver in the catalytically active silver (e.g. Cu, Pd, Pb, Bi, Fe, Pt and Au) is advantageously ≧30 ppm by weight, better ≧50 ppm by weight, preferably ≧100 ppm by weight and more preferably ≧1000 ppm by weight or ≧2000 ppm by weight. The longest dimension of the silver crystals is typically in the range from 0.1 to 5 mm and preferably increases in flow direction of reaction gas mixture A. The fixed silver bed is preferably configured as a two-layer bed, in which case the lower layer has a thickness, for example, of 15 to 40 mm, preferably 20 to 30 mm, and consists to an extent of at least 50% by weight of silver crystals of particle size 1 to 4 mm, preferably 1 to 2.5 mm. The upper layer may have, for example, a thickness (layer thickness) of 0.75 to 3 mm, preferably 1 to 2 mm, and consist of crystals having particle sizes (longest dimensions) of 0.1 to 1 mm, preferably 0.2 to 0.75 mm. In this case, reaction gas input mixture A flows in from the top downward.

In order to counteract sintering of the silver crystals with increasing operating time (at comparatively high reaction temperatures), which reduces the performance of the fixed catalyst bed, it is recommended to coat the silver crystals with a thin porous layer of oxidic material of at least one of the elements Al, Si, Zr and Ti (the layer thickness may be 0.3 to 10 μm, preferably 1.0 to 5.0 μm, more preferably 2.0 to 4.0 μm and at best about 3 μm), and in this way achieving prolonging of the service life of the fixed catalyst bed.

The methanol content in reaction gas input mixture A is, in the silver process, normally at least 5% by volume, usually at least 10% by volume, and may extend up to 60% by volume. The aforementioned methanol content in the silver process is preferably 15 to 50% by volume and more preferably 20 to 40 or to 30% by volume.

In addition, the ratio of the molar amount of molecular oxygen present in reaction gas input mixture A (nₒ) to the molar amount of methanol present in reaction gas input mixture A (n_{Me}), nₒ:n_{Me}, in the silver process is normally less than 1 (<1), preferably ≦0.8. It will more preferably range from 0.2 to 0.6 and most preferably 0.3 to 0.5 or 0.4 to 0.5. In one embodiment, nₒ:n_{Me} in the silver process is not less than 0.1.

In this document, an inert diluent gas shall be understood to mean a reaction gas input mixture constituent which behaves inertly under the conditions in the respective reaction zone A and/or B and, viewing each inert reaction gas constituent individually, remains chemically unchanged in the particular reaction zone to an extent of more than 95 mol %, preferably to an extent of more than 97 mol %, or to an extent of more than 98 mol %, or to an extent of more than 99 mol %.

Examples of inert diluent gases both for reaction zone A and reaction zone B are water, CO₂, N₂ and noble gases such as Ar, and mixtures of the aforementioned gases. One task assumed by the inert diluent gases is that of absorbing heat of reaction released in the reaction zone A, thus limiting what is called the hotspot temperature in reaction zone A and having a favorable effect on the ignition behavior of reaction gas mixture A. The hotspot temperature is understood to mean the highest temperature of reaction gas mixture A on its way through reaction zone A.

A preferred inert diluent gas other than steam in the case of the silver process for reaction gas input mixture A is molecular nitrogen. The advantage thereof may be based on the fact that molecular nitrogen occurs in air as a natural companion of molecular oxygen, which makes air a preferred source of the molecular oxygen required in reaction zone A. It will be appreciated that, in the case of the silver process, it is, however, also possible in accordance with the invention to use pure molecular oxygen, or air enriched with molecular oxygen, or another mixture of molecular oxygen and inert diluent gas, as the oxygen source.

Typically, reaction gas input mixture A comprises, in the case of the silver process, 20 to 80% by volume, or 30 to 70% by volume, or 40 to 60% by volume, of inert diluent gas. The latter may be entirely free of steam. In some embodiments, reaction gas input mixture A in the case of the silver process may comprise 20 to 80% by volume, or 30 to 70% by volume, or 40 to 60% by volume, of molecular nitrogen. In principle, reaction gas input mixture A in the case of the silver process may comprise >0 to 50% by volume of water.

Steam is advantageous as a constituent of reaction gas input mixture A in that steam, compared to N₂ and noble gases for example, has an increased molar heat capacity. In general, steam as a constituent of reaction gas mixture A is also beneficial for the desorption of the desired partial oxidation product from the catalyst surface, which has a positive effect on the selectivity of the desired product formation. Since presence of steam in reaction zone B, however, generally reduces the desired aldol condensation to a certain extent and also increases the energy expenditure required to remove a stream X comprising enriched acrylic acid from product gas mixture B in separation zone T (acrylic acid has an elevated affinity for water), appropriately in accordance with the invention, comparatively limited steam contents of reaction gas input mixture A are preferred.

In one embodiment, reaction gas input mixture A in the silver process preferably comprises from 5 to 45% by volume of water, advantageously from 10 to 40% by volume and particularly advantageously from 15 to 35% by volume, or from 20 to 30% by volume of water. The boiling point of the inert diluent gases other than steam (based on a pressure of 10⁵ Pa=1 bar) is normally well below that of steam (based on the same pressure), and therefore stream Z in the process according to the invention generally comprises the inert diluent gases other than steam, e.g., N₂ and CO₂ in enriched form. Advantageously in some embodiments, the separation of product gas mixture B in separation zone T will be performed in such a way that stream Z also has an appropriate content of steam. In the latter case, stream Z may function both as a source for inert gases other than steam and for steam. The inert gas source used in the silver process for reaction gas input mixture A may thus also be the stream Z obtained in separation zone T. Appropriately in one embodiment, in the silver process, a substream of stream Z will be recycled into reaction zone A to obtain reaction gas input mixture A (cycle gas method). It will be appreciated that a portion of stream Z may also be recycled into reaction zone B.

In some embodiments, suitable reaction gas input mixtures A may, in the silver process, comprise, for example, 10 to 50% by volume of water and 20 to 60% by volume of inert diluent gas other than steam (e.g. N₂, or N₂+CO₂, or N₂+noble gas (e.g. Ar), or N₂+CO₂+noble gas (e.g. Ar)).

It will be appreciated that reaction gas input mixtures A in the silver process may also comprise 10 to 40% by volume of water and 30 to 60% by volume of inert diluent gases other than steam (for example those mentioned above).

Of course, reaction gas input mixture A, in the silver process, may also comprise 20 to 40% by volume of water and 30 to 50% by volume of inert diluent gases other than steam (for example those mentioned above).

In principle, in the case of the silver process, reaction gas mixture A can be either forced or drawn through reaction zone A. Accordingly, the working pressure in the case of the silver process within reaction zone A may be either ≧10⁵ Pa or <10⁵ Pa. Appropriately in one embodiment, the working pressure in the case of the silver process in reaction zone A will be 10³ to 10⁶ Pa, preferably 10⁴ to 5×10⁵ Pa, more preferably 10⁴ to 2×10⁵ Pa and most preferably 0.5×10⁵ Pa to 1.8×10⁵ Pa.

The temperature of reaction gas mixture A (the term “reaction gas mixture A” comprises, in the present application, all gas mixtures which occur in reaction zone A and are between reaction gas input mixture A and product gas mixture A) will, in the case of the silver process, within reaction zone A, normally be within the range from 400 to 800° C., preferably within the range from 450 to 800° C. and more preferably within the range from 500 to 800° C. The term “temperature of reaction gas mixture A” (also referred to in this document as reaction temperature in reaction zone A) means primarily that temperature which reaction gas mixture A has from attainment of a conversion of the methanol present in reaction gas input mixture A of at least 5 mol % until attainment of the corresponding final conversion of the methanol within reaction zone A.

Advantageously in accordance with the invention, the temperature of reaction gas input mixture A in the case of the silver process is within the aforementioned temperature ranges over the entire reaction zone A.

Advantageously, in the case of the silver process, reaction gas input mixture A is also supplied to reaction zone A already with a temperature within the aforementioned range. Frequently, in the case of the silver process, a charge of reaction zone A with solid inert material or of catalytically active catalyst charge highly diluted with such inert material is present at the inlet into reaction zone A upstream in flow direction of the actually catalytically active catalyst charge (which may also be diluted with inert shaped bodies). As it flows through such an upstream charge of reaction zone A, the temperature of the reaction gas input mixture A supplied to reaction zone A in the case of the silver process can be adjusted comparatively easily to the value with which reaction gas mixture A in the case of the silver process is to enter the actual catalytically active catalyst charge of reaction zone A.

When the temperature of reaction gas mixture A in the case of the silver process within reaction zone A is limited to values of 450 to 650° C., preferably 500 to 600° C., the conversion of methanol will generally be ≦90 mol %, frequently ≦85 mol % or ≦80 mol %, while the selectivity of formaldehyde formation will be at values of ≧90 mol %, in many cases ≧93 mol % or ≧95 mol %. In this case (in which the steam content of the reaction gas input mixture is preferably <10% by volume), it is appropriate in accordance with the invention to remove from product gas mixture A at least a portion of unconverted methanol prior to the use thereof for obtaining reaction gas input mixture B, and to recycle it into the production of reaction gas input mixture A.

Advantageously in accordance with the invention, the temperature of reaction gas mixture A in the case of the silver process within reaction zone A will therefore be 550 to 800° C., preferably 600 to 750° C. and more preferably 650 to 750° C.

At the same time, the steam content of reaction gas input mixture A in the case of the silver process is advantageously adjusted to values of ≧10% by volume, preferably ≧15% by volume and particularly advantageously ≧20% by volume. Both the elevated temperature and the elevated steam content of reaction gas input mixture A, in the case of the silver process, have an advantageous effect on the methanol conversion (based on a single pass of reaction gas mixture A through reaction zone A). In general, this conversion will be >90 mol %, in many cases ≧92 mol %, or ≧95 mol % and frequently even ≧97 mol % (see, for example, Ullmann's Encyclopedia of Industrial Chemistry, vol. A 11, 5th ed., VCH Weinheim). The high methanol conversions which are to be achieved in the case of the silver process in spite of the comparatively low nₒ:n_{Me} ratios in reaction gas input mixture A are attributable in particular to the fact that, with increasing temperature of reaction gas mixture A in reaction zone A, the exothermic partial oxidation

CH₃OH+0.5O2→HCHO+water

is increasingly accompanied by the endothermic dehydration

CH₃OH [symbol] HCHO+H₂.

In this way, in the case of the silver process, it is regularly possible to achieve yields of formaldehyde of ≧85 mol %, usually ≧87 mol % and in many cases ≧89 mol % based on a single pass of reaction gas mixture A through reaction zone A and the molar amount of methanol converted. Otherwise, the silver process can be performed as described in the documents already mentioned in this regard, or as described in documents U.S. Pat. No. 4,080,383, U.S. Pat. No. 3,994,977, U.S. Pat. No. 3,987,107, U.S. Pat. No. 4,584,412 and U.S. Pat. No. 4,343,954. It will be appreciated that, in the case of the silver process described, it is possible not only to use comparatively pure methanol as the raw material (source). Methanol raw materials suitable in accordance with the invention in this regard are also aqueous methanol solutions and technical-grade methanol, which can be used after appropriate evaporation to obtain reaction gas input mixture A.

Suitable reactors for execution of the silver process in reaction zone A include not only those recommended in the aforementioned references but also heat exchanger reactors.

A heat exchanger reactor has at least one primary space and at least one secondary space, which are separated from one another by a dividing wall. The catalyst charge positioned in the at least one primary space comprises at least one oxidation catalyst A, and reaction gas mixture A flows through it. At the same time, a fluid heat carrier flows through the secondary space and heat exchange takes place between the two spaces through the dividing wall, which pursues the purpose of monitoring and controlling the temperature of reaction gas mixture A on its way through the catalyst bed (of controlling the temperature of reaction zone A).

Examples of heat exchanger reactors suitable in accordance with the invention for the implementation of reaction zone A are the tube bundle reactor (as disclosed, for example, in EP-A 700714 and the references cited in that document) and the thermoplate reactor (as disclosed, for example, in documents EP-A 1651344, DE-A 10361456, DE-A 102004017150 and the references acknowledged in these documents). In the case of the tube bundle reactor, the catalyst bed through which reaction gas mixture A flows is preferably within the tubes thereof (the primary spaces), and at least one heat carrier is conducted through the space surrounding the reaction tubes (the secondary space). Useful heat carriers for the heat exchanger reactors are, for example, salt melts, heat carrier oils, ionic liquids and steam. In general, tube bundle reactors used on the industrial scale comprise at least three thousand up to several tens of thousands of reaction tubes connected in parallel (reactor tubes). It will be appreciated that the configuration of reaction zone A can also be implemented in a fluidized bed reactor or a micro reactor.

Conventional reactors and micro reactors differ by their characteristic dimensions and especially by the characteristic dimensions of the reaction space which accommodates the catalyst bed through which the reaction gas mixture flows.

The space velocity of methanol present in reaction gas input mixture A on the reactor charged with silver crystals will generally be (0.5 to 6)×10³ kg of methanol per m² of reactor cross section or cross section of the fixed catalyst bed.

Preferably, in some embodiments, the heterogeneously catalyzed partial gas phase oxidation of methanol to formaldehyde in reaction zone A may be performed by the FORMOX process.

In contrast to the silver process, the FORMOX process is performed over oxidation catalysts A whose active material is a mixed oxide which has at least one transition metal in the oxidized state (see, for example, WO 03/053556 and EP-A 2213370). The term “transition metals” means the chemical elements of the Periodic Table with atomic numbers 21 to 30, 39 to 48 and 57 to 80.

Preferably, in accordance with the invention, aforementioned mixed oxide active materials comprise at least one of the transition metals Mo and V in the oxidized state. Most preferably in accordance with the invention, the aforementioned active materials are mixed oxides having at least the elements Fe and Mo in the oxidized state (see, for example, U.S. Pat. No. 3,983,073, U.S. Pat. No. 3,978,136, U.S. Pat. No. 3,975,302, U.S. Pat. No. 3,846,341, U.S. Pat. No. 3,716,497, U.S. Pat. No. 4,829,042, EP-A 2213370 and WO 2005/063375, U.S. Pat. No. 3,408,309, U.S. Pat. No. 3,198,753, U.S. Pat. No. 3,152,997, WO 2009/1489809, DE-A 2145851, WO 2010/034480, WO 2007/059974 and “Methanol Selective Oxidation to Formaldehyde over Iron-Molybdate Catalysts, Ana Paula Vieira Soares and Manuel Farinha Portela and Alain Kiennemann in Catalysis Review 47, pages 125 to 174 (2004)” and the references cited in these documents).

A further difference between the silver process and the FORMOX process is that the ratio of the molar amount of molecular oxygen present in reaction gas input mixture A (nₒ) to the molar amount of methanol present in reaction gas input mixture A (n_{Me}), nₒ:n_{Me}, is normally at least 1 or greater than 1 (≧1), preferably 1.1. In some embodiments, the nₒ:n_{Me} ratio in reaction gas input mixture A in the FORMOX process will, however, be not more than 5, frequently not more than 4. nₒ:n_{Me} ratios which are advantageous in accordance with the invention in reaction gas input mixture A are 1.5 to 3.5, preferably 2 to 3. An oxygen excess is advantageous in accordance with the invention in that, in the inventive procedure, the oxygen is introduced via product gas mixture A into reaction gas input mixture B, and hence into reaction zone B, which has an advantageous effect on the service life of the aldol condensation catalyst B. In addition, the methanol content of reaction gas input mixture A in the FORMOX process typically may be not more than 15% by volume, usually not more than 11% by volume because gas mixtures of molecular nitrogen, molecular oxygen and methanol with a molecular oxygen content of not more than approximately 11% by volume of molecular oxygen are outside the explosion range. In some embodiments, the methanol content in reaction gas input mixture A in the case of the FORMOX process will be 2% by volume, preferably 4 to 10% by volume and more preferably 6 to 9% by volume or 5 to 7% by volume. Gas mixtures of molecular nitrogen, molecular oxygen and methanol whose methanol content is ≦6.7% by volume are, irrespective of the molecular oxygen content therein, outside the explosion range, which is why particularly high nₒ:n_{Me} ratios in reaction gas input mixture A can be employed within this concentration range.

The FORMOX process also differs from the silver process in that the methanol conversions achieved by this process, based on a single pass of reaction gas mixture A through reaction zone A, essentially irrespective of the inert diluent gas used in reaction gas input mixture A, are regularly >90 mol %, typically ≧92 mol %, usually ≧95 mol % and in many cases even ≧97 mol % or ≧98 mol %, or ≧99 mol %. The accompanying selectivities of formaldehyde formation are regularly ≧90 mol %, usually ≧92 mol % and in many cases ≧94 mol %, and frequently even ≧96 mol %.

According to the invention, useful inert diluent gases in reaction gas input mixture A for the FORMOX process (and for the silver process) in reaction zone A are likewise gases such as water, N₂, CO₂ and noble gases such as Ar, and mixtures of aforementioned gases. A preferred inert diluent gas other than steam in the case of the FORMOX process too in reaction gas input mixture A is molecular nitrogen.

The inert diluent gas content in reaction gas input mixture A may, in the case of the FORMOX process, be 70 to 95% by volume, frequently 70 to 90% by volume and advantageously 70 to 85% by volume. In other words, the molecular nitrogen content of reaction gas input mixture A may, in the case of employment of the FORMOX process, in reaction gas input mixture A, be 70 to 95% by volume, or 70 to 90% by volume, or 70 to 85% by volume. Advantageously in accordance with the invention, reaction gas input mixture A in the case of the FORMOX process may be free of steam. Appropriately in application terms, reaction gas input mixture A, in the case of employment of a FORMOX process in reaction zone A, may have a low steam content for the same reasons as in the case of the silver process. In general, the steam content of reaction gas input mixture A in the FORMOX process in reaction zone A is ≧0.1% by volume and ≦20% by volume or ≦10% by volume, advantageously ≧0.2% by volume and ≦7% by volume, preferably ≧0.5% by volume and ≦5% by volume.

A further advantage of the employment of a FORMOX process in reaction zone A, in accordance with the invention, results from the fact that the high methanol conversions described are established at significantly lower reaction temperatures compared to the use of a silver process.

The temperature of reaction gas mixture A in the case of the FORMOX process in reaction zone A will normally be in the range from 250 to 500° C. preferably in the range from 300 to 450° C. and frequently within the range from 270 to 400° C. The meaning of the term “temperature of reaction gas mixture A” corresponds in the case of the FORMOX process to that which has already been given in this document for the silver process.

Advantageously in accordance with the invention, the temperature of reaction gas mixture A (also referred to in this document as the reaction temperature in reaction zone A) in the case of the FORMOX process, over the entire reaction zone A, is within the aforementioned temperature ranges. Advantageously, in the case of the FORMOX process too, reaction gas input mixture A is supplied to reaction zone A already with a temperature within the aforementioned range. Frequently, in the case of the FORMOX process, a charge of reaction zone A with solid inert material or of catalytically active catalyst charge highly diluted with such inert material is present at the inlet into reaction zone A upstream in flow direction of the actual catalytically active catalyst charge (which may also be diluted with inert shaped bodies). As it flows through such an upstream charge of reaction zone A, the temperature of reaction gas input mixture A supplied to reaction zone A in the FORMOX process can be adjusted in a comparatively simple manner to the value with which reaction gas mixture A in the FORMOX process is to enter the actual catalytically active catalyst charge of reaction zone A.

With regard to the working pressure in reaction zone A, the statements made with respect to the silver process may apply correspondingly to the FORMOX process.

Mixed oxide active materials particularly suitable for the FORMOX process are those of the general formula I

[Fe₂(MoO₄)₃]₁[M¹ₘOₙ]_{q} (I)

in which the variables are each defined as follows:

M¹ is Mo and/or Fe, or

Mo and/or Fe and a total molar amount, of up to 10 mol % (e.g. 0.01 to 10 mol %, or 0.1 to 10 mol %), preferably not more than 5 mol %, of one or more elements from the group consisting of Ti, Sb, Sn, Ni, Cr, Ce, Al, Ca, Mg, V, Nb, Ag, Mn, Cu, Co, Si, Na, K, Tl, Zr, W, Ir, Ta, As, P and B,

q is 0 to 5, or 0.5 to 3, or 1 to 2,

m is 1 to 3, and

n is 1 to 6, with the proviso that the contents of both sets of square brackets in Formula I are electrically uncharged, e.g., they do not have any electrical charge.

Advantageously, in accordance with the invention, mixed oxide active materials of formula I comprise less than 50 mol %, more preferably less than 20 mol % and more preferably less than 10 mol % of the Fe present in the mixed oxide active material of formula I in the +2 oxidation state, and the remaining amount of the Fe present therein in each case in the +3 oxidation state. Most preferably, the mixed oxide active material of formula I comprises all of the Fe present therein in the +3 oxidation state.

The n_{Mo}:n_{Fe} ratio of molar amount of Mo present in a mixed oxide active material of formula I (n_{Mo}) to molar amount of Fe present in the same mixed oxide active material (n_{Fe}) is preferably 1:1 to 5:1.

In addition, it is advantageous in accordance with the invention when M¹=Mo and m=1 and n=3. Mixed oxide active materials advantageous in accordance with the invention also exist when M¹=Fe and m=2 and n=3.

Favorable mixed oxide active materials of formula I favorable are also those with such a stoichiometry that they can be considered (represented) in a formal sense as a mixture of MoO₃ and Fe₂O₃, and the MoO₃ content of the mixture is 65 to 95% by weight and the Fe₂O₃ content of the mixture is 5 to 35% by weight.

Mixed oxide active materials of formula I can be prepared as described in the reference documents cited.

In general, the procedure will be to obtain, from sources of the catalytically active oxide material I, a very intimate, preferably finely divided, dry mixture of composition corresponding to the stoichiometry of the desired oxide material I (a precursor material), and to calcine (thermally treat) it at temperatures of 300 to 600° C. preferably 400 to 550° C. The calcination can be performed either under inert gas or under an oxidative atmosphere, for example air (or another mixture of inert gas and oxygen), or else under a reducing atmosphere (for example a mixture of inert gas and reducing gases such as NH₃ and CO). The calcination time will generally be a few hours and typically decreases with the magnitude of the calcination temperature.

Useful sources for the elemental constituents of the mixed oxide active materials I are especially those compounds which are already oxides and/or those compounds which can be converted to oxides by heating, at least in the presence of oxygen. The intimate mixing of the starting compounds (sources) can be performed in dry or in wet form. Where it is performed in dry form, the starting compounds are appropriately used in the form of fine powders and, after mixing and optional compaction, subjected to calcination. However, preference is given to performing the intimate mixing in wet form. In this case, the starting compounds are typically mixed with one another in the form of aqueous suspensions and/or solutions. Particularly intimate dry mixtures are obtained in the mixing process described when the starting materials are exclusively sources of the elemental constituents present in dissolved form.

The solvent used is preferably water. Preference is given to preparing, from the starting compounds, at least two aqueous solutions, at least one of which is an acidic solution and at least one of which is an ammoniacal (basic) solution.

Combination of the aqueous solutions generally results in precipitation reactions in which precursor compounds of the multimetal oxide active material I form.

Subsequently, the aqueous material obtained is dried, and the drying operation can be effected, for example, by spray drying.

The catalytically active oxide material obtained after the calcining of the dry material can be used to charge reaction zone A for the FORMOX process in finely divided form as such, or applied with the aid of a liquid binder to an outer surface of a shaped support body in the form of an eggshell catalyst. However, eggshell catalysts can also be produced by applying, with the aid of a liquid binder, fine precursor powder to the outer surface of shaped support bodies, and calcining the precursor substance only after completion of application and drying.

The multimetal oxide active materials of formula I can, however, also be used in reaction zone A in pure, undiluted form, or diluted with oxidic, essentially inert diluent material, in the form of what are called unsupported catalysts (this is preferred in accordance with the invention). Examples of inert diluent materials suitable in accordance with the invention include finely divided aluminum oxide, silicon dioxide, aluminosilicates, zirconium dioxide, titanium dioxide or mixtures thereof. Undiluted unsupported catalysts are preferred in accordance with the invention.

In the case of shaped unsupported catalyst bodies, the shaping is advantageously effected with precursor powder which is not calcined until after the shaping. The shaping is effected typically with addition of shaping aids, for example graphite (lubricant) or mineral fibers (reinforcing aid). Suitable shaping processes are tableting and extrusion. It will be appreciated that the shaping may, however, also be performed, for example, with a mixture of active material powder and precursor powder, to which shaping aids and optionally inert diluent powders are again added prior to the shaping. Shaping is followed by another calcination. In principle, the shaping to unsupported catalysts can also be performed only with already prefabricated active material powder and optionally the aids mentioned. The shaping here too is generally followed by another calcination.

A favorable Mo source is, for example, ammonium heptamolybdate tetrahydrate (NH₄)₆(Mo₇O₂₄).4H₂O. Advantageous iron sources are, for example, iron(III) nitrate [Fe(NO₃)₃], iron(III) chloride [FeCl₃] or hydrates of iron(III) nitrate, for example Fe(NO₃)₃.9H₂O.

Preferred geometries of the shaped support bodies for eggshell catalysts of the mixed oxide active materials of formula I are spheres and rings, the longest dimension of which is 1 to 10 mm, frequently 2 to 8 mm or 3 to 6 mm (the longest dimension of a shaped body in this document is generally understood to mean the longest direct line connecting two points on the surface of the shaped body).

Ring geometries favorable in accordance with the invention have hollow cylindrical shaped support bodies with a length of 2 to 10 mm, an external diameter of 4 to 10 mm and a wall thickness of 1 to 4 mm. The hollow cylindrical shaped support bodies preferably have a length of 3 to 6 mm, an external diameter of 4 to 8 mm and a wall thickness of 1 to 2 mm. In principle, the shaped support bodies may also have an irregular shape.

Suitable materials for the inert shaped support bodies are, for example, quartz, silica glass, sintered silica, sintered or fused alumina, porcelain, sintered or fused silicates such as aluminum silicate, magnesium silicate, zinc silicate, zirconium silicate, and especially steatite (e.g. C 220 steatite from CeramTec).

The inert shaped support bodies may differ from the catalytic active material normally in that they have a much lower specific surface area. In general, the specific surface area thereof is less than 3 m²/g of shaped support body. At this point, it should be emphasized that all figures in this document for specific surface areas relate to determinations according to DIN 66131 (determination of specific surface area of solids by means of gas absorption (N₂) according to Brunauer-Emmett-Teller (BET)).

The coating of the inert shaped support bodies with the particular finely divided powder is generally executed in a suitable rotatable vessel, for example in a coating drum. Appropriately, in some embodiments, the liquid binder is sprayed onto the inert shaped support bodies and the binder-moistened surface of the shaped support bodies being moved within the coating drum is dusted with the particular powder (see, for example, EP-A 714700). Subsequently, the adhering liquid is generally removed at least partly from the coated shaped support body (for example by passing hot gas through the coated shaped support bodies, as described in WO 2006/094765). In principle, however, it is also possible to employ all other application processes acknowledged as prior art in EP-A 714700 to produce the relevant eggshell catalysts. Useful liquid binders include, for example, water and aqueous solutions (for example of glycerol in water). For example, the coating of the shaped support bodies can also be undertaken by spraying a suspension of the pulverant material to be applied in liquid binder (for example water) onto the surface of the inert shaped support bodies (generally under the action of heat and a drying entraining gas). In principle, the coating can also be undertaken in a fluidized bed system or powder coating system.

The thickness of the eggshell of catalytically active oxide material applied to the surface of the inert shaped support body is, in the case of the mixed oxide active materials of formula I, appropriately in application terms, generally 10 to 1000 The eggshell thickness is preferably 10 to 500 μm, more preferably 100 to 500 μm and most preferably 200 to 300 μm. In one embodiment, suitable ring geometries for possible inert shaped support bodies of annular eggshell oxidation catalysts A for the inventive purposes in reaction zone A are all ring geometries disclosed in DE-A 102010028328 and in DE-A 102010023312, and all disclosed in EP-A 714700.

Preferred shaped unsupported catalyst bodies comprising mixed oxide active materials I are solid cylinders, hollow cylinders and trilobes. The external diameter of cylindrical unsupported catalysts is, appropriately in application terms, 3 to 10 mm, preferably 4 to 8 mm and in particular 5 to 7 mm.

The height thereof is advantageously 1 to 10 mm, preferably 2 to 6 mm and in particular 3 to 5 mm. The same applies in the case of hollow cylinders. In addition, the internal diameter of the orifice running through from the top downward is advantageously 1 to 8 mm, preferably 2 to 6 mm and most preferably 2 to 4 mm. Appropriately in some embodiments, the wall thickness of hollow cylinders is 1 to 3 mm.

In the case of shaped unsupported catalyst bodies (unsupported catalysts), the shaping can be performed, for example, in such a way that the pulverant active material or the uncalcined precursor material thereof (the latter being preferred in accordance with the invention) is used to directly produce unsupported catalysts or unsupported catalyst precursors by compaction (for example by tableting or extrusion) to the desired catalyst geometry. The shaping optionally may be preceded by addition of assistants, for example graphite or stearic acid as lubricants, and/or shaping assistants and reinforcing assistants such as microfibers of glass, asbestos, silicon carbide or potassium titanate. In the case of annular geometries, the tableting can advantageously be undertaken as described in documents WO 2008/152079, WO 2008/087116, DE-A 102008040094, DE-A 102008040093 and WO 2010/000720. All geometries detailed in the aforementioned documents are also suitable for inventive unsupported oxidation catalysts A.

The oxidation catalysts can, however, also be employed in reaction zone A as supported catalysts. In contrast to shaped support bodies for the eggshell oxidation catalysts A, which are preferably nonporous or low in pores, in the case of supported catalysts A, the active material is introduced into the pore structure of the shaped support bodies. In this case, the starting materials are therefore comparatively porous shaped support bodies which, for example, are impregnated successively with the at least two solutions of the precursor compounds. The precipitation reaction described proceeds in the pores of the shaped support body, and the precursor compounds which form therein can subsequently be converted to the desired mixed oxide active material I by calcination. Alternatively, it is also possible to impregnate with a solution comprising all sources required in dissolved form, to dry and then to calcine (see, for example, DE-A 2442311). Otherwise, the procedure for preparation of the mixed oxide active material I oxidation catalysts may be as in the reference documents to which reference is made in this regard in this application.

These are especially documents U.S. Pat. No. 3,716,497, U.S. Pat. No. 3,846,341, EP-A 199359, DE-A 2145851, U.S. Pat. No. 3,983,073, DE-A 2533209, EP-A 2213370 and Catalysis Review, 47, pages 125-174 (2004).

It will be appreciated that, in the FORMOX process, it is not only possible to use comparatively pure methanol to obtain reaction gas input mixture A. Methanol raw materials suitable in this regard in accordance with the invention are also aqueous methanol solutions and technical-grade methanol, which can be used after appropriate evaporation to obtain reaction gas input mixture A.

It is also possible to charge reaction zone A with a fixed catalyst bed which comprises FORMOX oxidation catalysts A in a form diluted with inert shaped bodies.

The space velocity on the fixed catalyst bed present in reaction zone A of reaction gas input mixture A will, in the case of a FORMOX process employed in accordance with the invention, generally be 3500 I (STP)/I·h to 75 000 I (STP)/I·h, preferably 25 000 I (STP)/I·h to 35 000 I (STP)/I·h. The term “space velocity” is used as defined in DE-A 19927624.

Suitable reactors for execution of the FORMOX process in reaction zone A are especially also the heat exchanger reactors which have already been recommended for implementation of reaction zone A in the case of the silver process (see, for example, WO 2005/063375).

In accordance with the invention, the FORMOX process is also preferred in reaction zone A because the product gas mixture A thereof, in contrast to a product gas mixture A after the silver process, is free of molecular hydrogen.

In other words, the product gas mixture A of a heterogeneously catalyzed partial gas phase oxidation of methanol to formaldehyde after the FORMOX process is, e.g., without subjecting it to a removal process beforehand, and/or without performing a removal process thereon beforehand, the ideal formaldehyde source for formaldehyde required in reaction gas input mixture B.

Frequently, product gas mixture A is obtained in the FORMOX process at a temperature at which it can be used without further thermal pretreatment for production of reaction gas input mixture B. In many cases, the temperature of the product gas mixture A leaving reaction zone A, both in the case of the silver process and in the case of the FORMOX process, however, is different from that temperature with which it is to be used to obtain reaction gas input mixture B. Against this background, the stream of product gas mixture A, on its way from reaction zone A into reaction zone B, can flow through an indirect heat exchanger in order to match its temperature to the addition temperature envisaged for production of reaction gas input mixture B.

For the sake of completeness, it should also be added that, in the case of employment of the FORMOX process in reaction zone A, the stream Z obtained in separation zone T in the process according to the invention may serve as a suitable inert gas source for the inert gas required in reaction gas input mixture A. In some embodiments, a substream of stream Z may be recycled into reaction zone A to obtain reaction gas input mixture A.

A useful source for the acetic acid required in reaction gas input mixture B for the process according to the invention is especially the carbonylation of methanol in the liquid phase:

CH₃OH+CO→CH₃COOH.

The reaction may be performed over a catalyst (homogeneous catalysis). Typically, the catalyst comprises at least one of the elements Fe, Co, Ni, Ru, Rh, Pd, Cu, Os, Ir and Pt, an ionic halide (e.g. KI) and/or a covalent halide (e.g. CH₃I) as a promoter (the iodides normally being the preferred promoters), and optionally a ligand, for example PR₃ or NR₃ where R is an organic radical. Corresponding carbonylation processes are disclosed, for example, in documents EP-A 1506151, DE 3889233 T2, EP-A 277824, EP-A 656811, DE-A 1941449, U.S. Pat. No. 6,420,304, EP-A 161874, U.S. Pat. No. 3,769,329, EP-A 55618, EP-A 87870, U.S. Pat. No. 5,001,259, U.S. Pat. No. 5,466,874 and U.S. Pat. No. 502,698, and the references cited in these documents. The working conditions require high pressures (at least 3 MPa (abs.)) and elevated temperatures (at least 150° C. or 250° C.). The catalyst system currently being employed preferentially in industrial scale processes is Rh in combination with HI/CH₃I as the promoter system (see DE 68916718 T2 and U.S. Pat. No. 3,769,329). The selectivities of acetic acid formation achieved, based on methanol converted, are ≧99 mol % (Industrielle Organische Chemie, Klaus Weissermel and Hans-Jurgen Arpe, Wiley-VCH, 5th edition, 1998, page 196 and Ullmann's Encyclopedia of Industrial Chemistry, Sixth Edition, volume 6 (2003)).

Since the liquid phase carbonylation of methanol, as described above, requires the additional use of halide promoters which have strongly corrosive action and require the use of expensive corrosion-resistant construction materials, the acetic acid formed is removed by rectification from the product mixture obtained in the carbonylation of methanol for use in the process according to the invention. This is typically accomplished in a purity of acetic acid content of at least 99.8% by weight (see Industrielle Organische Chemie, Klaus Weissermel and Hans-Jurgen Arpe, Wiley-VCH, 5th edition, 1998).

By conversion of the acetic acid that is removed by rectification to the gas phase (vapor phase) and combination with product gas mixture A or product gas mixture A*, it is possible, in a comparatively simple manner, to obtain the reaction gas input mixture B required for reaction zone B.

In principle, the carbonylation of methanol to acetic acid in the liquid phase can also be performed with exclusion of halide-comprising promoters (see for example, DE-A 3606169). In this case, the acetic acid present in the crude product of the carbonylation of methanol need not necessarily be removed therefrom by rectification in order to be able to be employed for production of reaction gas input mixture B. Instead, in this case, the crude product can also be converted as such to the vapor phase and used to obtain reaction gas input mixture B.

In one embodiment, the carbonylation of methanol with carbon monoxide may be performed in the gas phase, and the resulting product gas mixture comprising the acetic acid formed will be used directly to obtain reaction gas input mixture B.

In some preferred embodiments, heterogeneously catalyzed gas phase carbonylation processes of methanol to acetic acid, which do not require presence of halogen-containing promoters, will be employed. Exemplary gas phase carbonylations of methanol to acetic acid are disclosed by U.S. Pat. No. 4,612,387 and EP-A 596632. A characteristic feature of these processes is that the catalysts employed are zeolites (aluminosilicates) with anionic structural charge, which preferably have, on their inner and/or outer surfaces, at least one cation type from the group of the cations of the elements copper, iridium, nickel, rhodium and cobalt, in order to balance out (to neutralize) the negative structural charge. Particularly advantageous zeolites are those which have a mordenite structure (see Studies in Surface, Science and Catalysis, vol. 101, 11th International Congress on Catalysis—40th Anniversary), 1996, Elsevier, Science B. V., Lausanne).

It will be appreciated that the acetic acid source (the raw material) used for reaction gas input mixture B may also be an aqueous acetic acid solution or technical-grade acetic acid solution, which can be used after appropriate evaporation to obtain reaction gas input mixture B.

Reaction gas input mixture B can be obtained by combining the stream of product gas mixture A leaving reaction zone A, or the stream of product gas mixture A* leaving separation zone T* with the acetic acid source. The acetic acid source may be converted to the vapor phase. At least one further stream may also be combined to form the reaction gas mixture B. For example, stream Y, and optionally further streams, for example additional steam or additional inert diluent gas other than steam (also referred to in this document merely as inert gas for short) may be utilized. If required, for example when product gas mixture A does not comprise any excess molecular oxygen, reaction gas input mixture B can also be produced with additional use of molecular oxygen or a mixture of inert gas and molecular oxygen, since a low (limited) oxygen content in reaction gas input mixture B generally has an advantageous effect on the service life of aldol condensation catalyst B.

The temperature of reaction gas mixture B in the process according to the invention within reaction zone B will normally be within the range from 260 to 400° C. preferably within the range from 270 to 390° C. more preferably within the range of 280 to 380° C. advantageously within the range of 300 to 370° C. and particularly advantageously within the range of 300 to 340° C.

The term “temperature of reaction gas mixture B” (also referred to in this document as reaction temperature in reaction zone B) means primarily that temperature that reaction gas mixture B has from attainment of a conversion of the formaldehyde present in reaction gas input mixture B of at least 5 mol % until attainment of the appropriate final conversion of the formaldehyde within reaction zone B. Advantageously in accordance with the invention, the temperature of reaction gas mixture B over the entire reaction zone B is within the aforementioned temperature ranges. Advantageously, reaction gas input mixture B is already supplied to reaction zone B with a temperature within the range from 260 to 400° C. Frequently, however, a charge of reaction zone B with solid inert material or of catalytically active catalyst charge highly diluted with such inert material is present at the inlet into reaction zone B in flow direction upstream of the actual catalytically active catalyst charge of reaction zone B. As it flows through such a primary charge of reaction zone B, the temperature of the reaction gas input mixture B supplied to reaction zone B can be adjusted in a comparatively simple manner to the value with which reaction gas mixture B is to enter the actual catalytically active catalyst charge of reaction zone B. In general, the temperature of the product gas mixture A leaving reaction zone A is different than this temperature. In one embodiment, the stream of product gas mixture A, on its way from reaction zone A into reaction zone B, can flow through an indirect heat exchanger in order to approximate its temperature to the inlet temperature envisaged for reaction gas input mixture B into reaction zone B, or to bring it to this temperature.

In principle, the at least one aldol condensation catalyst B in reaction zone B can be configured in a fluidized bed. Advantageously in some embodiments, the aldol condensation catalyst B is, however, configured in a fixed bed.

With regard to the working pressure which exists in reaction zone B, the same applies correspondingly as has already been stated for the working pressure which exists in reaction zone A. In general, the working pressure in reaction zone B, due to the pressure drop which occurs as reaction gas mixture A flows through reaction zone A, is lower than the working pressure in reaction zone A. It is also possible to configure reaction zone B in corresponding heat exchanger reactors to reaction zone A, in which case the same ranges and limits apply.

The formaldehyde content in reaction gas input mixture B will, in the process according to the invention, generally be 0.5 to 10% by volume, preferably 0.5 to 7% by volume and more preferably 1 to 5% by volume.

The ratio n_{HAc}:n_{Fd} of molar amount of acetic acid present in reaction gas input mixture B (n_{HAc}) to molar amount of formaldehyde present therein (n_{Fd}) in the process according to the invention is greater than 1 and may be up to 10 (n_{Fd} is understood to mean the sum of formaldehyde units present in monomeric form (preferred) and possibly in oligomeric and polymeric form (formaldehyde has a tendency to such formations) in reaction gas input mixture B, since the latter undergo redissociation to monomeric formaldehyde under the reaction conditions in reaction zone B). Advantageously in accordance with the invention, the ratio n_{HAc}:n_{Fd} in reaction gas input mixture B is 1.1 to 5 and more preferably 1.5 to 3.5. Frequently, the acetic acid content of reaction gas input mixture B will vary within the range from 1 or from 1.5 to 20% by volume, advantageously within the range from 2 to 15% by volume and particularly advantageously within the range from 3 to 10% by volume. The molecular oxygen content of reaction gas input mixture B varies, in the process according to the invention, appropriately in application terms, within the range from 0.5 to 5% by volume, preferably within the range from 1 to 5% by volume and more preferably within the range from 2 or from 3 to 5% by volume. Presence of molecular oxygen in reaction gas input mixture B has an advantageous effect on the service life of the catalyst charge of reaction zone B. When the oxygen content of reaction gas mixture B is too high, however, there is unwanted carbon oxide formation in reaction zone B. In principle, the molecular oxygen content in reaction gas input mixture B in the process according to the invention may, however, also be vanishingly small.

The steam content of reaction gas input mixture B in the process according to the invention should not exceed 30% by volume since the presence of steam in reaction gas mixture B has an unfavorable effect on the equilibrium position of the aldol condensation. Appropriately, in application terms, the steam content of reaction gas input mixture B will therefore generally not exceed 25% by volume and preferably not exceed 20% by volume. In general, the steam content of reaction gas input mixture B will be at least 0.5% or at least 1% by volume. Advantageously, the steam content of reaction gas input mixture B is 0.5 to 15% by volume and, taking account of the effect thereof and formation thereof in reaction zone A, in particular 1 to 10% by volume. The proportion by volume of inert diluent gases other than steam in reaction gas input mixture B will normally be at least 30% by volume. Preferably, the aforementioned inert gas content is at least 40% by volume or at least 50% by volume. In general, the proportion of inert diluent gas other than steam in reaction gas input mixture B will not exceed 95% by volume or usually 90% by volume. Particularly advantageously in application terms, reaction gas input mixture B comprises 60 to 90% by volume, particularly advantageously 70 to 80% by volume, of inert diluent gas other than steam. An inert diluent gas other than steam which is preferred in accordance with the invention is also, in reaction gas input mixture B, molecular nitrogen (N₂).

In some embodiments, the molecular nitrogen content of reaction gas input mixture B may be at least 30% by volume, preferably at least 40% by volume or at least 50% by volume. In one embodiment, reaction gas input mixture B comprises not more than 95% by volume and usually not more than 90% by volume of molecular nitrogen. Advantageously, reaction gas input mixture B comprises 60 to 90% by volume, particularly advantageously 70 to 80% by volume, of molecular nitrogen.

### V—Bi Catalyst

In one embodiment, the present invention utilizes a catalyst composition comprising vanadium and bismuth. In one embodiment, the catalyst composition comprises a metal phosphate matrix and the matrix contains vanadium and bismuth. The catalyst composition may be substantially free of titanium. Surprisingly and unexpectedly, the vanadium-bismuth catalyst provides high conversions, selectivities, and yields when employed in an aldol condensation reaction.

The inventive catalyst composition has a low deactivation rate and provides stable performance over time, e.g., over 0.8 hours, over 3 hours, over 5 hours, over 10 hours, over 25 hours, over 60 hours, or over 100 hours, when utilized in the aldol condensation reaction.

In one embodiment, vanadium and bismuth are present as respective oxides or phosphates or mixtures thereof. The catalyst composition may comprise an active phase, which comprises the components that promote the catalysis, and may also comprise a support or a modified support. As one example, the active phase comprises metals, phosphorus-containing compounds, and oxygen-containing compounds. In a preferred embodiment, vanadium and bismuth are present in the active phase. Preferably, the molar ratio of vanadium to bismuth in the active phase of the catalyst composition is at least 0.02:1, e.g., at least 0.1:1, at least 0.25:1, at least 0.5:1, or at least 2:1. In terms of ranges, the molar ratio of vanadium to bismuth in the inventive catalyst may range from 0.02:1 to 1000:1, e.g., from 0.1:1 to 10:1, from 0.25:1 to 4:1, or from 0.5:1 to 2:1.

In one embodiment, the catalyst composition is substantially free of titanium, e.g., in the active phase, e.g. the catalyst composition comprises less than 5.0 wt % titanium, e.g., less than 1.0 wt %, or less than 0.1 wt %.

The inventive catalyst has been found to achieve unexpectedly high acetic acid conversions. For example, depending on the temperature at which the acrylate product, e.g., acrylic acid, formation reaction is conducted, alkanoic acid conversions of at least 20 mol %, e.g., at least 30 mol %, at least 40 mol %, or at least 50 mol %, may be achieved with this catalyst composition. This increase in acetic acid conversion is achieved while maintaining high selectivity to the desired acrylate product such as acrylic acid or methyl acrylate. For example, selectivities to the desired acrylate product of at least 35 mol %, e.g., at least 45 mol %, at least 60 mol %, at least 70 mol %, or at least 75 mol %, may be achieved with the catalyst composition of the present invention. Acrylate product yield is calculated by multiplying alkanoic acid conversion and acrylate product selectivity. Acrylate product yield may be greater than 7%, greater than 13.5%, greater than 24%, greater than 30%, or greater than 35%.

The total amounts of vanadium and bismuth in the catalyst composition of the invention may vary widely. In some embodiments, for example, the catalyst composition comprises in the active phase at least 0.3 wt % vanadium, e.g., at least 1.5 wt %, at least 3.5 wt %, at least 6 wt % or at least 10 wt %, based on the total weight of the active phase of the catalyst composition. The catalyst composition may comprise in the active phase at least 0.1 wt % bismuth, e.g., at least 10 wt %, at least 20 wt %, at least 30 wt % or at least 45 wt % bismuth. In terms of ranges, the catalyst composition may comprise in the active phase from 0.3 to 40 wt % vanadium, e.g., from 0.3 to 35 wt %, from 0.3 wt % to 32.5 wt %, from 1.0 wt % to 32 wt % or from 5 wt % to 20 wt %; and/or 0.1 wt % to 75 wt % bismuth, e.g., from 11 wt % to 70 wt % or from 20 wt % to 65 wt %, from 30 wt % to 60 wt %, from 0.1 to 10 wt %, from 0.3 to 5 wt %, or from 0.5 to 3 wt %. The catalyst composition may comprise in the active phase at most 32 wt % vanadium, e.g., at most 28 wt %, e.g., at most 23 wt % or at most 18 wt %. The catalyst composition may comprise in the active phase at most 75 wt % bismuth, e.g., at most 68 wt %, e.g., at most 64 wt %, at most 58 wt % or at most 50 wt %. In one embodiment, the catalyst comprises in the active phase vanadium and bismuth, in combination, in an amount greater than 25 wt %, e.g., greater than 35 wt %, greater than 40 wt %, greater than 50 wt %, or greater than 55 wt %. In terms of ranges, the combined weight percentage of the vanadium and bismuth, in combination in the active phase may range from 25 wt % to 75 wt %, e.g., from 35 wt % to 70 wt %, from 42 wt % to 68 wt %, or from 47 wt % to 64 wt %.

In other embodiments, the inventive catalyst may further comprise other compounds or elements (metals and/or non-metals). For example, the catalyst may further comprise phosphorus and/or oxygen. In these cases, the catalyst may comprise in the active phase from 7 wt % to 25 wt % phosphorus, e.g., from 10 wt % to 21 wt %, from 10 wt % to 21 wt %, or from 11 wt % to 19 wt %; and/or from 15 wt % to 55 wt % oxygen, e.g., from 18 wt % to 45 wt %, or from 25 wt % to 36 wt %.

In some embodiments, the bismuth is present in the form of a bismuth salt, including bismuth (III) and (V) salts. For example, the catalyst composition may comprise the bismuth salt in an amount ranging from 0.1 wt % to 75 wt %, e.g., from 11 wt % to 70 wt %, from 20 wt % to 65 wt %, or from 30 wt % to 60 wt %. Preferably the bismuth salt used in the preparation of the inventive catalyst is a bismuth (III) salt. The bismuth salt may for instance be selected from bismuth carboxylates, bismuth halides, bismuth acetate, bismuth sulphadiazine, bismuth sulphate, bismuth nitrate, bismuth subnitrate, bismuth carbonate, bismuth subcarbonate, bismuth oxide, bismuth oxychloride, bismuth hydroxide, bismuth phosphate, bismuth aluminate, bismuth tribromophenate, bismuth thiol, bismuth peptides, bismuth salts of quinolines and their derivatives,e.g., bismuth hydroxyquinolines, bismuth pyrithione and other bismuth salts of pyridine thiols, bismuth amino acid salts such as the glycinate, tripotassium dicitrato bismuthate, and mixtures thereof. In some embodiments, acid solutions such as nitric acid or acetic acid may be used to dissolve the bismuth salt to form a bismuth solution.

Generally speaking the bismuth salt may be either organic or inorganic. It may be a basic bismuth salt (bismuth subsalt) such as the subsalts referred to above.

Suitable bismuth carboxylates include the salicylate, subsalicylate, lactate, citrate, subcitrate, ascorbate, acetate, dipropylacetate, tartrate, sodium tartrate, gluconate, subgallate, benzoate, laurate, myristate, palmitate, propionate, stearate, undecylenate, aspirinate, neodecanoate and ricinoleate. Of these, basic bismuth salicylate (bismuth subsalicylate) and bismuth citrate may be preferred. Suitable halides include bismuth chloride, bismuth bromide and bismuth iodide. Preferred bismuth salts may be selected from bismuth halides, bismuth nitrates, bismuth acetate, and bismuth carboxylates, such as bismuth subsalicylate, bismuth salicylate, bismuth subgallate, bismuth subcitrate, bismuth citrate, bismuth nitrate and bismuth subnitrate.

In one embodiment, the formation of the catalyst composition may utilize the reduction of a pentavalent vanadium compound. The reduced pentavalent compound may be combined with a phosphorus compound and, optionally, promoters under conditions effective to provide or maintain the vanadium in a valence state below +5 to form the active metal phosphate catalysts. Various reducing agents and solvents may be used to prepare these catalysts. Examples include organic acids, alcohols, polyols, aldehydes, and hydrochloric acid. Generally speaking, the choice of the metal precursors, reducing agents, solvents, sequence of addition, reaction conditions such as temperature and times, and calcination temperatures may impact the catalyst composition, surface area, porosity, structural strength, and overall catalyst performance.

In one embodiment, suitable vanadium compounds that serve as a source of vanadium in the catalyst composition contain pentavalent vanadium and include, but are not limited to, vanadium pentoxide or vanadium salts such as ammonium metavanadate, vanadium oxytrihalides, vanadium alkylcarboxylates and mixtures thereof.

In one embodiment, suitable phosphorus compounds that serve as a source of phosphorus in the catalyst contain pentavalent phosphorus and include, but are not limited to, phosphoric acid, phosphorus pentoxide, polyphosphoric acid, or phosphorus perhalides such as phosphorus pentachloride, and mixtures thereof.

In one embodiment, the active phase of the catalyst corresponds to the formula:

VₐBi_{b}P_{c}O_{d},

wherein:
a is 1 to 100,
b is from 0.1 to 50,
c is from 1 to 165, and
d is from 4 to 670.

The letters a, b, c, and d are the relative molar amounts (relative to 1.0) of vanadium, bismuth, phosphorus and oxygen, respectively in the catalyst. In these embodiments, the ratio of a to b is greater than 0.02:1, e.g., greater than 5:1, greater than 10:1, or greater than 20:1. Preferred ranges for molar variables a, b, c, and d are shown in Table 1.

TABLE 1

| | Molar Range | Molar Range | Molar Range |
|---|---|---|---|
| a | 1 to 100 | 1 to 10 | 1 to 4 |
| b | 0.1 to 50 | 0.5 to 10 | 1 to 4 |
| c | 1 to 165 | 1 to 16 | 1 to 7 |
| d | 4 to 670 | 5 to 60 | 5 to 25 |

V—Ti—Bi catalyst

In another embodiment, the present invention utilizes a catalyst composition comprising a metal phosphate matrix containing vanadium, titanium, and bismuth. Surprisingly and unexpectedly, the vanadium-titanium-bismuth catalyst provides high conversions, selectivities, and yields when employed in an aldol condensation reaction.

The inventive catalyst composition also provides a low deactivation rate and provides stable performance for the aldol condensation reaction over a long period of time, e.g., over 5 hours, over 10 hours, over 20 hours, or over 50 hours.

In one embodiment, the vanadium, titanium and bismuth are present either in the elemental form or as a respective oxide or phosphate. The catalyst composition may comprise an active phase, which comprises the components that promote the catalysis, and may also comprise a support or a modified support. As one example, the active phase comprises metals, phosphorus-containing compounds, and oxygen-containing compounds. In a preferred embodiment, vanadium, titanium, and bismuth are present in the active phase. Preferably, the molar ratio of vanadium to bismuth in the active phase of the catalyst composition is greater than 0.2:1, e.g., greater than 0.4:1, or greater than 1:1, greater than 7:1, greater than 10:1, greater than 30:1, or greater than 62.5:1. In terms of ranges, the molar ratio of vanadium to bismuth in the active phase of the catalyst composition may range from 0.2:1 to 1000:1, e.g., from 0.5:1 to 250:1, from 1:1 to 62.5:1, from 2:1 to 62.5:1, from 10:1 to 62.5:1, from 37.5:1 to 62.5:1. In terms of upper limits, the molar ratio of vanadium to bismuth in the active phase of the catalyst composition is at most 1000:1, at most 250:1, at most 150:1, or at most 62.5:1. In an embodiment, the molar ratio of bismuth to titanium in the active phase of the catalyst composition is greater than 0.002:1, e.g., greater than 0.016:1, greater than 0.25:1, greater than 1:1, greater than 4:1, or greater than 6.25:1. In terms of ranges, the molar ratio of bismuth to titanium in the active phase of the catalyst composition may range from 0.002:1 to 500:1, e.g., from 0.016:1 to 150:1, from 0.25:1 to 100:1, 0.4:1 to 6.25:1; 1:1 to 6.25:1, or 2.5:1 to 6.25. In terms of upper limits, the molar ratio of bismuth to titanium in the active phase of the catalyst composition is at most 500:1, at most 150:1, at most 100:1, or at most 6.25:1. In an embodiment, the molar ratio of vanadium to titanium in the active phase of the catalyst composition is greater than 0.2:1, e.g., greater than 0.5:1, greater than 1:1, greater than 1.5:1, greater than 2.5:1, or greater than 62.5:1. In terms of ranges, the molar ratio of vanadium to titanium in the active phase of the catalyst composition may range from 0.2:1 to 1000:1, e.g., from 1:1 to 500:1, from 1.5:1 to 150:1, from 1.5:1 to 62.5:1, from 1.5:1 to 2:1, or from 2.5:1 to 62.5:1. Surprisingly and unexpectedly, the vanadium-titanium-bismuth catalyst having at least some of the ratios discussed above provides high conversions, selectivities, and yields when employed in an aldol condensation reaction.

The inventive catalyst has been found to achieve unexpectedly high acetic acid conversions. For example, depending on the temperature at which the acrylic acid formation reaction is conducted, acetic acid conversions of at least 15 mol %, e.g., at least 25 mol %, at least 30 mol %, at least 40 mol %, or at least 50 mol %, may be achieved with this catalyst composition. This increase in acetic acid conversion is achieved while maintaining high selectivity to the desired acrylate product such as acrylic acid or methyl acrylate. For example, selectivities to the desired acrylate product of at least 35 mol %, e.g., at least 45 mol % or at least 60 mol % may be achieved with the catalyst composition of the present invention.

The total amounts of vanadium, titanium and bismuth in the catalyst composition of the invention may vary widely. In some embodiments, for example, the catalyst composition comprises at least 0.1 wt. % vanadium, e.g., at least 0.15 wt %, at least 0.2 wt. %, at least 0.4 wt. %, at least 0.9 wt. %, at least 1.7 wt. %, at least 2 wt. %, at least 6.8 wt. %, or at least 8.5 wt. % based on the total weight of the active phase of the catalyst composition. The catalyst composition may comprise in the active phase at least 0.015 wt. % titanium, e.g., at least 0.09 wt. %, at least 0.36 wt. %, at least 0.41 wt. %, or at least 3.2 wt. %. The catalyst composition may comprise in the active phase at least 0.07 wt. % bismuth, e.g., at least 0.15 wt. %, at least 0.4 wt. %, at least 0.8 wt. %, at least 1.2 wt. %, or at least 1.8 wt. % based on the total weight of the active phase of the catalyst composition. In terms of ranges, the catalyst composition may comprise in the active phase from 0.15 wt. % to 32 wt. % vanadium, e.g., from 0.4 wt. % to 28 wt. %, from 0.9 wt. % to 28 wt. %, or from 2 wt % to 27 wt %; from 0.015 wt. % to 22 wt. % titanium, e.g., from 0.03 wt. % to 20 wt. %, from 0.09 wt. % to 19 wt. %, from 0.3 wt. % to 15 wt. %, or from 0.3 wt. % to 11.09 wt. %; and 0.07 wt. % to 70 wt. % bismuth, e.g., from 0.15 wt. % to 69 wt. %, from 0.4 wt. % to 66 wt. %, 0.8 wt. % to 35 wt. %, or from 0.8 wt. % to 34 wt. %. The catalyst composition may comprise at most 32 wt. % vanadium, e.g., at most 30 wt. % or at most 28 wt. %. The catalyst composition may comprise in the active phase at most 22 wt. % titanium, e.g., at most 20 wt. % or at most 19 wt. %. The catalyst composition may comprise in the active phase at most 70 wt. % bismuth, e.g., at most 69 wt. % or at most 66 wt. %.

In one embodiment, the catalyst comprises in the active phase vanadium and titanium, in combination, in an amount greater than 0.3 wt. %, e.g., greater than 0.4 wt. % greater than 0.7 wt. %, greater than 1.8 wt. %, greater than 5 wt. % or greater than 10 wt. %. In terms of ranges, the combined weight percentage of the vanadium and titanium components in the active phase may range from 0.4 wt. % to 32 wt. %, e.g., from 0.7 wt. % to 30 wt. %, from 1.8 wt. % to 28 wt. %, from 5 wt. % to 28 wt %, or from 10 wt. % to 28 wt. %. In one embodiment, the catalyst comprises in the active phase vanadium and bismuth, in combination, in an amount greater than 0.6 wt. %, e.g., greater than 1.2 wt. %, greater than 2.8 wt. %, greater than 5 wt. % or greater than 13 wt. %. In terms of ranges, the combined weight percentage of the vanadium and bismuth, in combination in the active phase may range from 0.6 wt. % to 72 wt. %, e.g., from 1.2 wt. % to 70 wt. %, from 2.8 wt. % to 68 wt. %, from 5 wt. % to 40 wt. % or fro 10 wt. % to 28 wt. %. In one embodiment, the catalyst comprises in the active phase bismuth and titanium, in combination, in an amount greater than 0.15 wt. %, e.g., greater than 0.3 wt. %, greater than 1.0 wt. %, or greater than 2.0 wt. %. In terms of ranges, the combined weight percentage of the bismuth and titanium components in the active phase may range from 0.15 wt. % to 70 wt. %, e.g., from 0.3 wt. % to 69 wt. %, from 1.0 wt. % to 66 wt. %, or from 2.0 wt. % to 42 wt %. In one embodiment, the catalyst comprises in the active phase vanadium, titanium, bismuth, in combination, in an amount greater than 20 wt. %, e.g., greater than 22 wt. %, greater than 24 wt. %, or greater than 25 wt. %. In terms of ranges, the combined weight percentage of the vanadium, titanium and bismuth components in the active phase may range from 20 wt. % to 72 wt. %, e.g., from 22 wt. % to 69 wt. %, from 24 wt. % to 46 wt. %, or from 25 wt. % to 66 wt. %.

In other embodiments, the inventive catalyst may further comprise other compounds or elements (metals and/or non-metals). For example, the catalyst may further comprise phosphorus and/or oxygen. In these cases, the catalyst may comprise from 10 wt. % to 30 wt. % phosphorus, e.g., from 11 wt. % to 28 wt. %; and/or from 19 wt. % to 55 wt. % oxygen, e.g., from 20 wt. % to 51 wt. % or from 21 wt. % to 51 wt. %.

In some embodiments, the bismuth is present in the form of a bismuth salt, including bismuth (III) and (V) salts. For example, the catalyst composition may comprise the bismuth salt in an amount ranging from 0.07 wt. % to 70 wt. %, e.g., from 0.15 wt. % to 69 wt. % or from 0.4 wt. % to 66 wt. %. Preferably the bismuth salt used in the preparation of the inventive catalyst is a bismuth (III) salt. The bismuth salt may for instance be selected from bismuth carboxylates, bismuth halides, bismuth acetate, bismuth sulphadiazine, bismuth sulphate, bismuth nitrate, bismuth subnitrate, bismuth carbonate, bismuth subcarbonate, bismuth oxide, bismuth oxychloride, bismuth hydroxide, bismuth phosphate, bismuth aluminate, bismuth tribromophenate, bismuth thiol, bismuth peptides, bismuth salts of quinolines and their derivatives (e.g., bismuth hydroxyquinolines), bismuth pyrithione and other bismuth salts of pyridine thiols, bismuth amino acid salts such as the glycinate, tripotassium dicitrato bismuthate, and mixtures thereof.

Generally speaking the bismuth salt may be either organic or inorganic. It may be a basic bismuth salt (bismuth subsalt) such as the subsalts referred to above.

Suitable bismuth carboxylates include the salicylate, subsalicylate, lactate, citrate, subcitrate, ascorbate, acetate, dipropylacetate, tartrate, sodium tartrate, gluconate, subgallate, benzoate, laurate, myristate, palmitate, propionate, stearate, undecylenate, aspirinate, neodecanoate and ricinoleate. Of these, basic bismuth salicylate (bismuth subsalicylate) and bismuth citrate may be preferred. Suitable halides include bismuth chloride, bismuth bromide and bismuth iodide. Preferred bismuth salts may be selected from bismuth halides, bismuth nitrates, bismuth acetate, and bismuth carboxylates, such as bismuth subsalicylate, bismuth salicylate, bismuth subgallate, bismuth subcitrate, bismuth citrate, bismuth nitrate and bismuth subnitrate.

In one embodiment, the formation of the catalyst composition may utilize the reduction of a pentavalent vanadium compound. The reduced pentavalent compound may be combined with a phosphorus compound and, optionally, promoters under conditions effective to provide or maintain the vanadium in a valence state below +5 to form the active metal phosphate catalysts. Various reducing agents and solvents may be used to prepare these catalysts. Examples include organic acids, alcohols, polyols, aldehydes, and hydrochloric acid. Generally speaking, the choice of the metal precursors, reducing agents, solvents, sequence of addition, reaction conditions such as temperature and times, and calcination temperatures may impact the catalyst composition, surface area, porosity, structural strength, and overall catalyst performance.

In one embodiment, suitable vanadium compounds that serve as a source of vanadium in the catalyst composition contain pentavalent vanadium and include, but are not limited to, vanadium pentoxide or vanadium salts such as ammonium metavanadate, vanadium oxytrihalides, vanadium alkylcarboxylates and mixtures thereof.

In some embodiments, the titanium is present in compound form such as in the form of titanium dioxide. For example, the catalyst may comprise titanium dioxide in an amount ranging from 0.1 wt. % to 95 wt. %, e.g., from 5 wt. % to 50 wt. % or from 7 wt. % to 25 wt. %. In these cases, the titanium dioxide may be in the rutile and/or anatase form, with the anatase form being preferred. If present, the catalyst preferably comprises at least 5 wt. % anatase titanium dioxide, e.g., at least 10 wt. % anatase titanium dioxide, or at least 50 wt. % anatase titanium dioxide. Preferably less than 20 wt. % of the titanium dioxide, if present in the catalyst, is in rutile form, e.g., less than 10 wt. % or less than 5 wt. %. In other embodiments, the catalyst comprises anatase titanium dioxide in an amount of at least 5 wt. %, e.g., at least 10 wt. % or at least 20 wt. %. In another embodiment, the titanium is present in the form of amorphous titanium hydroxide gel, which is preferably converted to TiP₂O₇.

The titanium hydroxide gel may be prepared by any suitable means including, but not limited to, the hydrolysis of titanium alkoxides, substituted titanium alkoxides, or titanium halides. In other embodiments, colloidal titania sols and/or dispersions may be employed. In one embodiment, titania coated colloidal particles or supports are used as a source of titanium dioxide. The hydrous titania may be amorphous or may contain portions of anatase and/or rutile depending on preparation method and heat treatment.

Upon treatment with a phosphating agent, the various forms of titania may be converted to titanium phosphates and/or titanium pyrophosphates. In some cases, a portion of the titanium may be present as unconverted titania and, hence, will be present in the final catalyst as anatase or rutile forms.

Generally speaking, the proportion of the crystalline forms of titania present in the catalyst is dependent on the titanium precursor, the preparative method, and/or the post-phosphorylating treatment. In one embodiment, the amount of anatase and rutile present in the active phase of the catalyst is minimized. The amount of crystalline titania, however, may be high with only a thin shell of porous catalyst existing on the titania support.

In one embodiment, suitable phosphorus compounds that serve as a source of phosphorus in the catalyst contain pentavalent phosphorus and include, but are not limited to, phosphoric acid, phosphorus pentoxide, polyphosphoric acid, or phosphorus perhalides such as phosphorus pentachloride, and mixtures thereof.

In one embodiment, the active phase of the catalyst corresponds to the formula:

VₐBi_{b}Ti_{c}P_{d}Oₑ,

wherein:
a is 1 to 100,
b is from 0.1 to 50,
c is from 0.1 to 50,
d is from 1.5 to 270, and
e is from 6 to 1045.

The letters a, b, c, d and e are the relative molar amounts (relative to 1.0) of vanadium, bismuth, titanium, phosphorus and oxygen, respectively in the catalyst. In these embodiments, the ratio of a to b is greater than 0.2:1, e.g., greater than 0.4:1, or greater than 1:1 and the ratio of a to c is greater than 0.2:1, e.g., greater than 0.5:1, greater than 1:1, greater than 1.5:1, greater than 2.5:1, or greater than 62.5:1. Preferred ranges for molar variables a, b, c, d and e are shown in Table 2.

TABLE 2
Molar Ranges

| | Molar Range | Molar Range | Molar Range |
|---|---|---|---|
| A | 1 to 100 | 1 to 50 | 2 to 10 |
| B | 0.1 to 50 | 0.1 to 25 | 0.1 to 10 |
| C | 0.1 to 50 | 0.1 to 25 | 0.1 to 10 |
| D | 1.5 to 270 | 1.5 to 135 | 1.5 to 49 |
| E | 6 to 1045 | 6.1 to 523 | 6 to 186 |

V—Bi—W catalyst

Accordingly, in still another embodiment, the present invention utilizes a catalyst composition comprising vanadium, bismuth, and tungsten. Surprisingly and unexpectedly, the vanadium-bismuth-tungsten catalyst provides high conversions, selectivities, and yields. The inventive catalyst composition also shows a low deactivation rate and provides stable performance for the aldol condensation reaction over a long periods of time.

The inventive catalyst composition has a low deactivation rate and provides stable performance for the aldol condensation reaction over a long period of time, e.g., over 50 hours, over 77 hours, or over 100 hours.

In one embodiment, vanadium, bismuth and tungsten are present either as a respective oxides or phosphates or mixtures thereof. The catalyst composition may comprise an active phase, which comprises the components that promote the catalysis, and may also comprise a support or a modified support. As one example, the active phase comprises metals, phosphorus-containing compounds, and oxygen-containing compounds. In a preferred embodiment, vanadium, bismuth, and tungsten are present in the active phase.

The inventive catalyst has been found to achieve unexpectedly high alkanoic acid, e.g., acetic acid, conversions. For example, depending on the temperature at which the acrylic acid formation reaction is conducted, acetic acid conversions of at least 15 mol %, e.g., at least 25 mol %, at least 30 mol %, e.g., at least 40 mol %, or at least 50 mol %, may be achieved with this catalyst composition. This increase in acetic acid conversion is achieved while maintaining high selectivity to the desired acrylate product such as acrylic acid or methyl acrylate. For example, selectivities to the desired acrylate product of at least 35 mol %, e.g., a least 45 mol %, at least 60 mol % or at least 75 mol % may be achieved with the catalyst composition of the present invention.

The total amounts of vanadium, bismuth and tungsten in the catalyst composition of the invention may vary widely. In some embodiments, for example, the catalyst composition comprises in the active phase at least 0.3 wt % vanadium, e.g., at least 0.6 wt %, or at least 1.6 wt %, based on the total weight of the active phase of the catalyst composition. The catalyst composition may comprise in the active phase at least 0.1 wt % bismuth, e.g., at least 0.5 wt %, at least 1 wt %, at least 3 wt %, or at least 8 wt %. The catalyst composition may comprise in the active phase at least 0.1 wt % tungsten, e.g., at least 0.5 wt %, at least 1 wt %, at least 2.5 wt %, or at least 3.7 wt %. In terms of ranges, the catalyst composition may comprise in the active phase from 0.3 wt % to 30 wt % vanadium, e.g., from 0.6 wt % to 25 wt % or from 1.6 wt % to 20 wt %; from 0.1 wt % to 69 wt % bismuth, e.g., from 3 wt % to 64 wt % or from 8 wt % to 58 wt %; and 0.1 wt % to 61 wt % tungsten, e.g., from 2.5 wt % to 59 wt % or from 3.7 wt % to 49 wt %. The catalyst composition may comprise in the active phase at most 30 wt % vanadium, e.g., at most 25 wt % or at most 20 wt %. The catalyst composition may comprise in the active phase at most 69 wt % bismuth, e.g., at most 64 wt % or at most 58 wt %. The catalyst composition may comprise in the active phase at most 61 wt % tungsten, e.g., at most 59 wt % or at most 49 wt %.

In one embodiment, the catalyst comprises in the active phase vanadium and bismuth, in combination, in an amount at least 0.79 wt %, e.g., at least 1 wt %, at least 3 wt %, at least 5.6 wt % or at least 14 wt %. In terms of ranges, the combined weight percentage of the vanadium and bismuth components in the active phase may range from 0.79 wt % to 70 wt %, e.g., from 5.6 wt % to 65 wt %, or from 14 wt % to 61 wt %. In one embodiment, the catalyst comprises in the active phase vanadium and tungsten, in combination, in an amount at least 0.76 wt %, e.g., at least 1 wt %, at least 3 wt %, at least 4.8 wt % or at least 8 wt %. In terms of ranges, the combined weight percentage of the vanadium and tungsten components in the active phase may range from 0.76 wt % to 62 wt %, e.g., from 4.8 wt % to 60 wt % or from 8 wt % to 52 wt %.

In one embodiment, the molar ratio of vanadium to bismuth in the active phase of the catalyst composition is at least 0.033:1, e.g., at least 0.20:1, at least 1:1, at least 2:1, at least 10:1, or at least 50:1. In terms of ranges, the molar ratio of vanadium to bismuth in the active phase of the catalyst composition may range from 0.033:1 to 1000:1, e.g., from 0.2:1 to 500:1, from 1:1 to 250:1, from 2:1 to 100:1, from 2:1 to 65:1, or from 10:1 to 65:1. In terms of upper limits, the molar ratio of vanadium to bismuth in the active phase of the catalyst composition is at most 1000:1, e.g., at most 500:1, at most 250:1, at most 100:1, or at most 65:1. In one embodiment, the molar ratio of bismuth to tungsten in the active phase of the catalyst composition is at least 0.0033:1, e.g., at least 0.067:1, at least 0.1:1, at least 0.20:1, at least 0.32:1, at least 0.75:1, at least 1.5:1, or at least 3:1. In terms of ranges, the molar ratio of bismuth to tungsten in the active phase of the catalyst composition may range from 0.0033:1 to 300:1, e.g., from 0.033 to 100:1, from 0.067:1 to 50:1, from 0.20:1 to 10:1, or from 0.32:1 to 5:1. In terms of upper limits, the molar ratio of bismuth to tungsten in the active phase of the catalyst composition is at most 300:1, e.g., at most 150:1, at most 75:1, at most 15:1, at most 10:1, or at most 5:1. In one embodiment, the molar ratio of vanadium to tungsten in the active phase of the catalyst composition is at least 0.033:1, e.g., at least 0.067:1, at least 0.20:1, at least 1:1, at least 10:1, or at least 50:1. In terms of ranges, the molar ratio of vanadium to tungsten in the active phase of the catalyst composition may range from 0.033:1 to 1000:1, e.g., from 0.067:1 to 500:1, from 0.1:1 to 250:1, from 1:1 to 100:1, or from 5:1 to 20:1. In terms of upper limits, the molar ratio of vanadium to tungsten in the active phase of the catalyst composition is at most 1000:1, e.g., at most 500:1, at most 250:1, at most 100:1, at most 50:1, at most 15:1, or at most 10:1.

In one embodiment, the catalyst composition is substantially free of titanium, e.g., in the active phase, e.g. comprises less than 5 wt % titanium, e.g., less than 1 wt %, or less than 0.1 wt %.

In other embodiments, the inventive catalyst may further comprise other compounds or elements (metals and/or non-metals). For example, the catalyst may further comprise phosphorus and/or oxygen in the active phase. In these cases, the catalyst may comprise in the active phase from 10 wt % to 22 wt % phosphorus, e.g., from 11 wt % to 20 wt % or from 11 wt % to 18 wt %; and/or from 15 wt % to 50 wt % oxygen, e.g., from 20 wt % to 45 wt % or from 22 wt % to 38 wt %.

In some embodiments, the bismuth is present in the form of a bismuth salt, including bismuth (III) and (V) salts. For example, the catalyst composition may comprise in the active phase the bismuth salt in an amount ranging from 0.1 wt % to 69 wt %, e.g., from 3 wt % to 64 wt % or from 8 wt % to 58 wt %. Preferably the bismuth salt used in the preparation of the inventive catalyst is a bismuth (III) salt. The bismuth salt may for instance be selected from bismuth carboxylates, bismuth halides, bismuth acetate, bismuth sulphadiazine, bismuth sulphate, bismuth nitrate, bismuth subnitrate, bismuth carbonate, bismuth subcarbonate, bismuth oxide, bismuth oxychloride, bismuth hydroxide, bismuth phosphate, bismuth aluminate, bismuth tribromophenate, bismuth thiol, bismuth peptides, bismuth salts of quinolines and their derivatives (e.g., bismuth hydroxyquinolines), bismuth pyrithione and other bismuth salts of pyridine thiols, bismuth amino acid salts such as the glycinate, tripotassium dicitrato bismuthate, and mixtures thereof. In some embodiments, acid solutions such as nitric acid or acetic acid may be used to dissolve the bismuth salt to form a bismuth solution.

Generally speaking the bismuth salt may be either organic or inorganic. It may be a basic bismuth salt (bismuth subsalt) such as the subsalts referred to above.

Suitable bismuth carboxylates include the salicylate, subsalicylate, lactate, citrate, subcitrate, ascorbate, acetate, dipropylacetate, tartrate, sodium tartrate, gluconate, subgallate, benzoate, laurate, myristate, palmitate, propionate, stearate, undecylenate, aspirinate, neodecanoate and ricinoleate. Of these, basic bismuth salicylate (bismuth subsalicylate) and bismuth citrate may be preferred. Suitable halides include bismuth chloride, bismuth bromide and bismuth iodide. Preferred bismuth salts may be selected from bismuth halides, bismuth nitrates, bismuth acetate, and bismuth carboxylates, such as bismuth subsalicylate, bismuth salicylate, bismuth subgallate, bismuth subcitrate, bismuth citrate, bismuth nitrate and bismuth subnitrate.

In some embodiments, the tungsten is present in the form of a tungsten salt. For example, the catalyst composition may comprise in the active phase the tungsten salt in an amount ranging from 0.1 wt % to 61 wt %, e.g., from 2.5 wt % to 59 wt % or from 3.7 wt % to 49 wt %. Preferably the tungsten salt used in the preparation of the inventive catalyst is tungsten (VI) salt. The tungsten salt may for instance be selected from tungstic acid, silicotungstic acid, ammonium silicotungstic acid, ammonium metatungstate hydrate, ammonium paratungstate, ammonium tetrathiotungstate, hydrogentungstate, polymer-supported, bis(tert-butylimino)bis(dimethylamino)tungsten(VI), phosphotungstic acid hydrate, piperidine tetrathiotungstate, tungsten(VI) chloride, tungsten(VI) dichloride dioxide, tungsten(VI) fluoride, tungsten(IV) oxide, tungsten(VI) oxychloride, tungstosilicic acid hydrate.

In one embodiment, the formation of the catalyst composition may utilize the reduction of a pentavalent vanadium compound. The reduced pentavalent compound may be combined with a phosphorus compound and, optionally, promoters under conditions effective to provide or maintain the vanadium in a valence state below +5 to form the active metal phosphate catalysts. Various reducing agents and solvents may be used to prepare these catalysts. Examples include organic acids, alcohols, polyols, aldehydes, and hydrochloric acid. Generally speaking, the choice of the metal precursors, reducing agents, solvents, sequence of addition, reaction conditions such as temperature and times, and calcination temperatures may impact the catalyst composition, surface area, porosity, structural strength, and overall catalyst performance.

In one embodiment, suitable vanadium compounds that serve as a source of vanadium in the catalyst composition contain pentavalent vanadium and include, but are not limited to, vanadium pentoxide or vanadium salts such as ammonium metavanadate, vanadium oxytrihalides, vanadium alkylcarboxylates and mixtures thereof.

In one embodiment, suitable phosphorus compounds that serve as a source of phosphorus in the catalyst contain pentavalent phosphorus and include, but are not limited to, phosphoric acid, phosphorus pentoxide, polyphosphoric acid, or phosphorus perhalides such as phosphorus pentachloride, and mixtures thereof.

In one embodiment, the active phase of the catalyst corresponds to the formula:

VₐBi_{b}W_{c}P_{d}Oₑ

wherein
a is from 1 to 100,
b is from 0.1 to 30,
c is from 0.1 to 30,
d is from 1.0 to 175, and
e is from 5 to 710.

The letters a, b, c, d and e are the relative molar amounts (relative to 1.0) of vanadium, bismuth, tungsten, phosphorus and oxygen, respectively in the catalyst. In these embodiments, the ratio of a to b is greater than 1:30, e.g., greater than 1:1, greater than 4:1, or greater than 10:1. Preferred ranges for molar variables a, b, c, d and e are shown in Table 3.

TABLE 3
Molar Ranges

| | Molar Range | Molar Range | Molar Range |
|---|---|---|---|
| a | 1 to 100 | 1 to 15 | 2 to 10 |
| b | 0.1 to 30 | 1 to 15 | 2 to 10 |
| c | 0.1 to 30 | 1 to 15 | 1 to 10 |
| d | 1 to 180 | 3 to 50 | 5 to 35 |
| e | 5 to 710 | 10 to 210 | 20 to 150 |

V—Ti—W catalyst

Accordingly, in yet another embodiment, the present invention is to a catalyst composition comprising a metal phosphate matrix containing vanadium, titanium, and tungsten. Surprisingly and unexpectedly, the vanadium-titanium-tungsten catalyst provides high conversions, selectivities, and yields, as compared to conventional catalysts.

The inventive catalyst composition also shows a low deactivation rate and provides stable performance for the aldol condensation reaction over a long period of time, e.g., over 5 hours, over 10 hours, over 20 hours, or over 50 hours.

In one embodiment, the vanadium, titanium and tungsten are present either in the elemental form or as a respective oxide or phosphate. The catalyst composition may comprise an active phase, which comprises the components that promote the catalysis, and may also comprise a support or a modified support. As one example, the active phase comprises metals, phosphorus-containing compounds, and oxygen-containing compounds. In a preferred embodiment, vanadium, titanium, and tungsten are present in the active phase. Preferably, the molar ratio of vanadium to tungsten in the active phase of the catalyst composition is greater than 0.02:1, e.g., greater than 0.05:1, greater than 0.10:1, greater than 1:1, greater than 7:1, greater than 10:1, or greater than 30:1, or greater than 62.5:1. In terms of ranges, the molar ratio of vanadium to tungsten in the inventive catalyst may range from 0.02:1 to 1000:1, e.g., from 0.05:1 to 250:1, from 0.10:1 to 150:1, or from 2:1 to 62.5:1. In an embodiment, the molar ratio of vanadium to titanium in the active phase of the catalyst composition is greater than 0.02:1, e.g., greater than 0.05:1, greater than 0.10:1, greater than 0.44:1, greater than 1:1, greater than 10:1, or greater than 30:1. In terms of ranges, the molar ratio of vanadium to titanium in the inventive catalyst may range from 0.02:1 to 1000:1, e.g., from 0.05:1 to 500:1, from 0.10:1 to 150:1, from 0.40:1 to 62.5:1.

The inventive catalyst has been found to achieve unexpectedly high acetic acid conversions. For example, depending on the temperature at which the acrylic acid formation reaction is conducted, acetic acid conversions of at least 15 mol %, e.g., at least 25 mol %, at least 30 mol %, e.g., at least 40 mol %, or at least 50 mol %, may be achieved with this catalyst composition. This increase in acetic acid conversion is achieved while maintaining high selectivity to the desired acrylate product such as acrylic acid or methyl acrylate. For example, selectivities to the desired acrylate product of at least 35 mol %, e.g., at least 45 mol %, at least 60 mol %, or at least 70 mol % may be achieved with the catalyst composition of the present invention.

The total amounts of vanadium, titanium and tungsten in the catalyst composition of the invention may vary widely. In some embodiments, for example, the catalyst composition comprises at least 0.2 wt % vanadium, e.g., at least 1.0 wt %, at least 6 wt %, at least 10 wt %, or at least 27 wt. % based on the total weight of the active phase of the catalyst composition. The catalyst composition may comprise in the active phase at least 0.016 wt % titanium, e.g., at least 0.24 wt %, at least 1 wt %, at least 8 wt %, or at least 13 wt %. The catalyst composition may comprise in the active phase at least 0.11 wt % tungsten, e.g., at least 0.4 wt %, at least 0.6 wt %, at least 2 wt %, at least 5 wt % or at least 9 wt %. In terms of ranges, the catalyst composition may comprise in the active phase from 0.2 wt % to 35 wt % vanadium, e.g., from 0.5 wt % to 29 wt %, from 1 wt % to 28 wt %, or from 6 wt % to 28 wt %; from 0.016 wt % to 25 wt % titanium, e.g., from 0.24 wt % to 25 wt %, from 0.27 wt % to 14 wt %, from 0.9 wt % to 19 wt %; and 0.11 wt % to 65 wt % tungsten, e.g., from 0.21 wt % to 63 wt %, from 0.4 wt % to 58 wt %, or from 0.6 wt % to 10 wt %. The catalyst composition may comprise at most 35 wt % vanadium, e.g., at most 28 wt % or at most 20 wt %. The catalyst composition may comprise in the active phase at most 25 wt % titanium, e.g., at most 19 wt % or at most 18 wt %. The catalyst composition may comprise in the active phase at most 65 wt % tungsten, e.g., at most 63 wt % or at most 58 wt %.

In one embodiment, the catalyst comprises in the active phase vanadium and titanium, in combination, in an amount at least 0.4 wt %, e.g., at least 3 wt %, at least 10 wt %, at least 15 wt %, at least 18 wt %, at least 20 wt %, or at least 29 wt %. In terms of ranges, the combined weight percentage of the vanadium and titanium components in the active phase may range from 0.4 wt % to 30 wt %, e.g., from 1.3 wt % to 30 wt %, from 3 wt % to 29 wt %, or from 18 wt % to 30 wt %. In one embodiment, the catalyst comprises in the active phase vanadium and tungsten, in combination, in an amount at least 0.58 wt %, e.g., at least 3 wt %, at least 9 wt %, at least 15 wt %, at least 25 wt %, or at least 33 wt %. In terms of ranges, the combined weight percentage of vanadium and tungsten in the active phase may range from 0.58 wt % to 65 wt %, e.g., from 1.4 wt % to 63 wt %, from 2.7 wt % to 5.9 wt %, or from 9 wt % to 34 wt %. In one embodiment, the catalyst comprises in the active phase vanadium, titanium and tungsten, in combination, in an amount at least 20 wt %, e.g., at least 23 wt %, at least 28 wt %, at least 33 wt %, or at least 38 wt %. In terms of ranges, the combined weight percentage of the vanadium, titanium and tungsten components in the active phase may range from 20 wt % to 65 wt %, e.g., from 21 wt % to 64 wt %, from 22 wt % to 61 wt %, or from 23 wt % to 39 wt %.

In other embodiments, the inventive catalyst may further comprise other compounds or elements (metals and/or non-metals). For example, the catalyst may further comprise phosphorus and/or oxygen. In these cases, the catalyst may comprise from 12 wt % to 21 wt % phosphorus, e.g., from 13 wt % to 28 wt % or from 14 wt % to 28 wt %; and/or from 22 wt % to 51 wt % oxygen, e.g., from 23 wt % to 51 wt % or from 26 wt % to 51 wt %.

In some embodiments, the tungsten is present in the form of a tungsten salt. For example, the catalyst composition may comprise the tungsten salt in an amount ranging from 0.1 wt % to 65 wt %, e.g., from 0.21 wt % to 63 wt % or from 0.4 wt % to 58 wt %. Preferably the tungsten salt used in the preparation of the inventive catalyst is tungsten (VI) salt. The tungsten salt may for instance be selected from tungstic acid, silicotungstic acid, ammonium silicotungstic acid, ammonium metatungstate hydrate, ammonium paratungstate, ammonium tetrathiotungstate, hydrogentungstate, polymer-supported, bis(tert-butylimino)bis(dimethylamino)tungsten(VI), phosphotungstic acid hydrate, piperidine tetrathiotungstate, tungsten(VI) chloride, tungsten(VI) dichloride dioxide, tungsten(VI) fluoride, tungsten(IV) oxide, tungsten(VI) oxychloride, tungstosilicic acid hydrate.

In one embodiment, the formation of the catalyst composition may utilize the reduction of a pentavalent vanadium compound. The reduced pentavalent compound may be combined with a phosphorus compound and, optionally, promoters under conditions effective to provide or maintain the vanadium in a valence state below +5 to form the active metal phosphate catalysts. Various reducing agents and solvents may be used to prepare these catalysts. Examples include organic acids, alcohols, polyols, aldehydes, and hydrochloric acid. Generally speaking, the choice of the metal precursors, reducing agents, solvents, sequence of addition, reaction conditions such as temperature and times, and calcination temperatures may impact the catalyst composition, surface area, porosity, structural strength, and overall catalyst performance.

In one embodiment, suitable vanadium compounds that serve as a source of vanadium in the catalyst composition contain pentavalent vanadium and include, but are not limited to, vanadium pentoxide or vanadium salts such as ammonium metavanadate, vanadium oxytrihalides, vanadium alkylcarboxylates and mixtures thereof.

In some embodiments, the titanium is present in compound form such as in the form of titanium dioxide. For example, the catalyst may comprise titanium dioxide in an amount ranging from 0.1 wt % to 95 wt %, e.g., from 5 wt % to 50 wt % or from 7 wt % to 25 wt %. In these cases, the titanium dioxide may be in the rutile and/or anatase form, with the anatase form being preferred. If present, the catalyst preferably comprises at least 5 wt % anatase titanium dioxide, e.g., at least 10 wt % anatase titanium dioxide, or at least 50 wt % anatase titanium dioxide. Preferably less than 20 wt % of the titanium dioxide, if present in the catalyst, is in rutile form, e.g., less than 10 wt % or less than 5 wt %. In other embodiments, the catalyst comprises anatase titanium dioxide in an amount of at least 5 wt %, e.g., at least 10 wt % or at least 20 wt %. In another embodiment, the titanium is present in the form of amorphous titanium hydroxide gel, which is preferably converted to TiP₂O₇.

The titanium hydroxide gel may be prepared by any suitable means including, but not limited to, the hydrolysis of titanium alkoxides, substituted titanium alkoxides, or titanium halides. In other embodiments, colloidal titania sols and/or dispersions may be employed. In one embodiment, titania coated colloidal particles or supports are used as a source of titanium dioxide. The hydrous titania may be amorphous or may contain portions of anatase and/or rutile depending on preparation method and heat treatment.

Upon treatment with a phosphating agent, the various forms of titania may be converted to titanium phosphates and/or titanium pyrophosphates. In some cases, a portion of the titanium may be present as unconverted titania and, hence, will be present in the final catalyst as anatase or rutile forms.

Generally speaking, the proportion of the crystalline forms of titania present in the catalyst is dependent on the titanium precursor, the preparative method, and/or the post-phosphorylating treatment. In one embodiment, the amount of anatase and rutile present in the active phase of the catalyst is minimized. The amount of crystalline titania, however, may be high with only a thin shell of porous catalyst existing on the titania support.

In one embodiment, suitable phosphorus compounds that serve as a source of phosphorus in the catalyst contain pentavalent phosphorus and include, but are not limited to, phosphoric acid, phosphorus pentoxide, polyphosphoric acid, or phosphorus perhalides such as phosphorus pentachloride, and mixtures thereof.

In one embodiment, the active phase of the catalyst corresponds to the formula:

VₐTi_{b}W_{c}P_{d}Oₑ

wherein:
a is from 1 to 100,
b is from 0.1 to 50,
c is from 0.1 to 50,
d is from 1 to 270,
e is from 6 to 1040.

The letters a, b, c, d and e are the relative molar amounts (relative to 1.0) of vanadium, titanium, tungsten, phosphorus and oxygen, respectively in the catalyst. In these embodiments, the ratio of a to b is greater than 0.02:1, e.g., greater than 0.05:1, greater than 0.10:1, greater than 1:1, or greater than 2:1. Preferred ranges for molar variables a, b, c, d and e are shown in Table 4.

TABLE 4
Molar Ranges

| | Molar Range | Molar Range | Molar Range |
|---|---|---|---|
| a | 1 to 100 | 1 to 25 | 1 to 15 |
| b | 0.1 to 50 | 0.1 to 20 | 0.1 to 10 |
| c | 0.1 to 50 | 0.1 to 20 | 0.1 to 10 |
| d | 1 to 270 | 2 to 91 | 3 to 50 |
| e | 6 to 1040 | 9 to 344 | 13 to 184 |

In some embodiments, the catalyst composition further comprises additional metals and/or metal oxides. These additional metals and/or metal oxides may function as promoters. If present, the additional metals and/or metal oxides may be selected from the group consisting of copper, molybdenum, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,364,824, the entirety of which is incorporated herein by reference.

If the catalyst composition comprises additional metal(s) and/or metal oxides(s), the catalyst optionally may comprise in active phase additional metals and/or metal oxides in an amount from 0.001 wt % to 30 wt %, e.g., from 0.01 wt % to 5 wt % or from 0.1 wt % to 5 wt %. If present, the promoters may enable the catalyst to have a weight/weight space time yield of at least 25 grams of acrylic acid/gram catalyst-h, e.g., at least 50 grams of acrylic acid/gram catalyst-h, or at least 100 grams of acrylic acid/gram catalyst-h.

In some embodiments, the catalyst composition is unsupported. In these cases, the catalyst may comprise a homogeneous mixture or a heterogeneous mixture as described above. In one embodiment, the homogeneous mixture is the product of an intimate mixture of the catalyst metals resulting from preparative methods such as controlled hydrolysis of metal alkoxides or metal complexes. In other embodiments, the heterogeneous mixture is the product of a physical mixture of the metal salt(s). These mixtures may include formulations prepared from phosphorylating a physical mixture of preformed hydrous metal oxides. In other cases, the mixture(s) may include a mixture of preformed metal pyrophosphate powders.

In another embodiment, the catalyst composition is a supported catalyst comprising a catalyst support in addition to the catalyst metals and optionally phosphorous and oxygen, in the amounts indicated above (wherein the molar ranges indicated are without regard to the moles of catalyst support, including any catalyst metals, phosphorous or oxygen contained in the catalyst support). The total weight of the support (or modified support), based on the total weight of the catalyst, preferably is from 25 wt % to 95 wt %, e.g., from 40 wt % to 70 wt % or from 50 wt % to 60 wt %, and the total weight of the active phase is from 0.1 wt % to 25 wt %, based on the total weight of the catalyst composition. In a preferred embodiment, the weight of the active phase is at least 6 wt % of the total catalyst composition weight.

The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, and mixtures thereof. Other supports may include, but are not limited to, iron oxide, magnesia, steatite, magnesium oxide, carbon, graphite, high surface area graphitized carbon, activated carbons, and mixtures thereof. Other supports may include coated structured forms such as coated metal foil, sintered metal forms and coated ceramic formed shapes such as shaped cordierite, platy alumina or acicular mullite forms. These listings of supports are merely exemplary and are not meant to limit the scope of the present invention.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt % to 50 wt %, e.g., from 0.2 wt % to 25 wt %, from 0.5 wt % to 15 wt %, or from 1 wt % to 8 wt %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

In some embodiments in which a support is employed, the support may have a surface area of at least 1 m²/g, e.g., at least 20 m²/g or at least 50 m²/g, as determined by BET measurements. The catalyst support may include pores, optionally having an average pore diameter ranging from 5 nm to 200 nm, e.g., from 5 nm to 50 nm or from 10 nm to 25 nm. The catalyst optionally has an average pore volume of from 0.05 cm³/g to 3 cm³/g, e.g., from 0.05 cm³/g to 0.1 cm³/g or from 0.08 cm³/g to 0.1 cm³/g, as determined by BET measurements. Preferably, at least 50% of the pore volume or surface area, e.g., at least 70% or at least 80%, is provided by pores having the diameters discussed above. Pores may be formed and/or modified by pore modification agents, which are discussed below. In another embodiment, the ratio of microporosity to macroporosity ranges from 19:1 to 5.67:1, e.g., from 3:1 to 2.33:1. Microporosity refers to pores smaller than 2 nm in diameter, and movement in micropores may be described by activated diffusion. Mesoporosity refers to pores greater than 2 nm and less than 50 nm is diameter. Flow through mesopores may be described by Knudson diffusion. Macroporosity refers to pores greater than 50 nm in diameter and flow though macropores may be described by bulk diffusion. Thus, in some embodiments, it is desirable to balance the surface area, pore size distribution, catalyst or support particle size and shape, and rates of reaction with the rate of diffusion of the reactant and products in and out of the pores to optimize catalytic performance.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, methyl cellulose, silica, alumina, glass fibers, silicon carbide, and silicon nitride. In one embodiment, the active phase of the catalyst (not the support) comprises the other additives. For example, the active phase may comprise silica, e.g., colloidal silica. In such embodiments, the silica may be present in the active phase in amounts ranging from 0.01 to 50 wt % silica, e.g., from 0.1 wt % to 40 wt %, from 0.5 wt % to 30 wt %, from 1.0 wt % to 30 wt %, from 2 wt % to 15 wt %, or from 2 wt % to 9 wt %. In terms of lower limits, the active phase may comprise at least 0.01 wt % silica, e.g., at least 0.1 wt %, at least 0.5 wt %, or at least 1 wt %. In terms of upper limits, the active phase may comprise less than 50 wt % silica, e.g., less than 40 wt %, less than 30 wt %, or less than 20 wt %. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

In one embodiment, the inventive catalyst composition comprises a pore modification agent. In some embodiments, the pore modification agent may be thermally stable and has a substantial vapor pressure at a temperature below 300° C., e.g., below 250° C. In one embodiment, the pore modification agent has a vapor pressure of at least 0.1 kPa, e.g., at least 0.5 kPa, at a temperature between about 150° C. and about 250° C., e.g., between about 150° C. and about 200° C. In other embodiments, pore modification agent may be thermally decomposed or burned out to create pores. For example, the burned out agent may be cellulose-derived materials such as ground nut shells.

In some embodiments, the pore modification agent has a relatively high melting point, e.g., greater than 60° C., e.g., greater than 75° C., so that it does not melt during compression of the catalyst precursor into a slug, tablet, or pellet. Preferably, the pore modification agent comprises a relatively pure material rather than a mixture. As such, lower melting components will not liquefy under compression during formation of slugs or tablets. For example, where the pore modification agent is a fatty acid, lower melting components of the fatty acid mixtures may be removed as liquids by pressing. If this phenomenon occurs during slug or tablet compression, the flow of liquid may disturb the pore structure and produce an undesirable distribution of pore volume as a function of pore diameter on the catalyst composition. In other embodiments, the pore modification agents have a significant vapor pressure at temperatures below their melting points, so that they can be removed by sublimination into a carrier gas.

For example, the pore modification agent may be a fatty acid corresponding to the formula CH₃(CH₂)ₓCOOH where x>8. Exemplary fatty acids include stearic acid (x=16), palmitic acid (x=14), lauric acid (x=10), myristic acid (x=12). The esters of these acids and amides or other functionalized forms of such acids, for example, stearamide (CH₃(CH₂)₁₆CONH₂) may also be used. Suitable esters may include methyl esters as well as glycerides such as stearin (glycerol tristearate). Mixtures of fatty acids may be used, but substantially pure acids, particularly stearic acid, are generally preferred over mixtures.

In addition, while fatty acids and fatty acid derivatives are generally preferred, other compositions which meet the functional requirements discussed above are also suitable for use as pore modification agents. Other preferred pore modification agents include but are not limited to polynuclear organic compounds such as naphthalene, graphite, natural burnout components such as cellulose and its cellulosic derivatives, cellulose-derived materials, such as starches and ground nut shells, such as walnut powder, natural and synthetic oligomers and polymers such as polyethylene, polyvinyl alcohols and polyacrylic acids and esters.

Examples of suitable catalyst compositions are disclosed in U.S. patent application Ser. Nos. 13/792,814, 13/664,494, 13/664,477, and 13/664,478, which are hereby incorporated by reference.

The process for forming the aldol condensation catalysts described hereinbefore, namely, the V—Bi, V—Ti—B, V—Bi—W and V—Ti—W catalysts, may further comprise the step of mixing the vanadium precursor with a reductant solution to form the vanadium precursor solution. In one embodiment, the reductant solution may comprise an acid, silica, water, and/or a glycol. In one embodiment the acid may be an organic acid that may be oxidized by vanadium, e.g., V⁵⁺. In an embodiment, the acid may be selected from the group consisting of citric acid, oxalic acid, steric acid, maleic acid, lactic acid, tartaric acid, glycol acid, pyruvic acid, polyacrylatic acid and mixtures thereof. In one embodiment, the acid utilized in the reductant solution does not comprise acids that are not oxidized by vanadium, e.g., V⁵⁺, e.g., formic acid, acetic acid, succinic acid, and mixtures thereof. In an embodiment, the glycol may be selected from the group consisting of propylene glycol, ethylene glycol, diethylene glycol, triethylene glycol, and other polyols. Preferably, the reductant solution comprises an organic acid, e.g., citric acid and/or oxalic acid, colloidal silica, deionized water, and ethylene glycol. In other embodiments, the reductant solution may also comprise ketones, aldehydes, alcohols, and phenols.

In one embodiment, the formation of the wet catalyst precursor also includes the addition of a binder. Thus, the contacting step may comprise contacting the binder, e.g., a binder solution, with the non-vanadium active phase element salt(s) and/or the vanadium precursor solution to form the wet catalyst composition. In one embodiment, the binder may be selected from the group consisting of cellulose, methyl cellulose, carboxyl methyl cellulose, cellulose acetate, starch, walnut powder, and combinations of two or more of the foregoing polysaccharides. In one embodiment, the catalyst composition comprises at least 3 wt. % of the binder, e.g., at least 5 wt. % or at least 10 wt. %. In one embodiment, an acid, e.g., phosphoric acid, may be added to the wet catalyst composition.

Advantageously, in one embodiment of the present invention, bismuth precursor is added to the wet catalyst mixture prior to phosphorylation, i.e., addition of phosphoric acid solution. In this manner, the bismuth can be incorporated into the metal phosphate matrix of the active phase of the catalyst.

The process, in one embodiment, may further comprise calcining the dried catalyst, which, preferably, is conducted in accordance with a temperature profile. As one example, the temperature profile comprises an increasing stair step temperature profile comprising a plurality of increasing hold temperatures. The temperature increases at a rate from 1° C. to 10° C. per minute between said hold temperatures. Preferably, the hold temperatures comprise a first, second, third, and fourth hold temperature. The first hold temperature may range from 150° C. and 300° C., e.g., from 175° C. and 275° C., preferably being about 160° C. The second hold temperature may range from 250° C. and 500° C., e.g., from 300° C. and 400° C., preferably being about 250° C. The third hold temperature may range from 300° C. and 700° C., e.g., from 450° C. and 650° C., preferably being about 300° C. The fourth hold temperature may range from 400° C. and 700° C., e.g., from 450° C. and 650° C., preferably being about 450° C. Of course, other temperature profiles may be suitable. The calcination of the mixture may be done in an inert atmosphere, air or an oxygen-containing gas at the desired temperatures. Steam, a hydrocarbon or other gases or vapors may be added to the atmosphere during the calcination step or post-calcination to cause desired effects on physical and chemical surface properties as well as textural properties such as increase macroporosity.

In one preferred embodiment, the temperature profile comprises:
i) heating the dried catalyst from room temperature to 160° C. at a rate of 10° C. per minute;
ii) heating the dried catalyst composition at 160° C. for 2 hours;
iii) heating the dried catalyst composition from 160° C. to 250° C. at a rate of 3° C. per minute;
iv) heating the dried catalyst composition at 250° C. for 2 hours;
v) heating the dried catalyst composition from 250° C. to 300° C. at a rate of 3° C. per minute;
vi) heating the dried catalyst composition at 300° C. for 6 hours;
vii) heating the dried catalyst composition from 300° C. to 450° C. at a rate of 3° C. per minute; and
viii) heating the dried catalyst composition at 450° C. for 6 hours.

In one embodiment the metal oxides and/or phosphates precursors are physically mixed, milled, or kneaded and then calcined to form the active catalyst.

In one embodiment the phosphorylating agent is added to the mixed metal oxides precursors followed by calcinations.

In one embodiment the catalyst is prepared under hydrothermal conditions followed by calcinations.

In embodiments where the catalyst is supported, the catalyst compositions are formed through metal impregnation of a support (optionally modified support), although other processes such as chemical vapor deposition may also be employed.

In one embodiment, the catalysts are made by impregnating the support, with a solution of the metals or salts thereof in a suitable solvent, followed by drying and optional calcination. Solutions of the modifiers or additives may also be impregnated onto the support in a similar manner. The impregnation and drying procedure may be repeated more than once in order to achieve the desired loading of metals, modifiers, and/or other additives. In some cases, there may be competition between the modifier and the metal for active sites on the support. Accordingly, it may be desirable for the modifier to be incorporated before the metal. Multiple impregnation steps with aqueous solutions may reduce the strength of the catalyst particles if the particles are fully dried between impregnation steps. Thus, it is preferable to allow some moisture to be retained in the catalyst between successive impregnations. In one embodiment, when using non-aqueous solutions, the modifier and/or additive are introduced first by one or more impregnations with a suitable non-aqueous solution, e.g., a solution of an alkoxide or acetate of the modifier metal in an alcohol, e.g., ethanol, followed by drying. The metal may then be incorporated by a similar procedure using a suitable solution of a metal compound.

In other embodiments, the modifier is incorporated into the composition by co-gelling or co-precipitating a compound of the modifier element with the silica, or by hydrolysis of a mixture of the modifier element halide with a silicon halide. Methods of preparing mixed oxides of silica and zirconia by sol gel processing are described by Bosman, et al., in J Catalysis, Vol. 148, (1994), page 660 and by Monros et al., in J Materials Science, Vol. 28, (1993), page 5832. Also, doping of silica spheres with boron during gelation from tetraethyl orthosilicate (TEOS) is described by Jubb and Bowen in J Material Science, Vol. 22, (1987), pages 1963-1970. Methods of preparing porous silicas are described in Iler R K, The Chemistry of Silica, (Wiley, New York, 1979), and in Brinker C J & Scherer G W Sol-Gel Science published by Academic Press (1990).

The catalyst composition, in some embodiments, will be used in a fixed bed reactor for forming the desired product, e.g., acrylic acid or alkyl acrylate. Thus, the catalyst is preferably formed into shaped units, e.g., spheres, granules, pellets, powders, aggregates, or extrudates, typically having maximum and minimum dimensions in the range of 1 to 25 mm, e.g., from 2 to 15 mm. Where an impregnation technique is employed, the support may be shaped prior to impregnation. Alternatively, the composition may be shaped at any suitable stage in the production of the catalyst. The catalyst also may be effective in other forms, e.g. powders or small beads and may be used in these forms. In one embodiment, the catalyst is used in a fluidized bed reactor. In this case, the catalyst may be prepared via spray drying or spray thermal decomposition. Preferably, the resultant catalyst has a particle size of greater than 300 microns, e.g., greater than 500 microns.

In some embodiments, the catalyst compositions described hereinbefore further comprise additional metals and/or metal oxides. These additional metals and/or metal oxides may function as promoters. If present, the additional metals and/or metal oxides may be selected from the group consisting of copper, molybdenum, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BP₃, ZnBr₂, and SnCl₄. Exemplary processes for incorporating promoters into catalyst are described in U.S. Pat. No. 5,364,824, the entirety of which is incorporated herein by reference.

If the catalyst compositions comprise additional metal(s) and/or metal oxides(s), the catalysts optionally may comprise additional metals and/or metal oxides in an amount from 0.001 wt % to 30 wt %, e.g., from 0.01 wt % to 5 wt % or from 0.1 wt % to 5 wt %. If present, the promoters may enable the catalysts to have a weight/weight space time yield of at least 25 grams of acrylic acid/gram catalyst-h, e.g., at least 50 grams of acrylic acid/gram catalyst-h, or at least 100 grams of acrylic acid/gram catalyst-h.

In some embodiments, the catalyst compositions are unsupported. In these cases, the catalysts may comprise a homogeneous mixture or a heterogeneous mixture as described above. In one embodiment, the homogeneous mixture is the product of an intimate mixture of vanadium and bismuth resulting from preparative methods such as controlled hydrolysis of metal alkoxides or metal complexes. In other embodiments, the heterogeneous mixture is the product of a physical mixture of the active phase element salts. These mixtures may include formulations prepared from phosphorylating a physical mixture of preformed hydrous metal oxides. In other cases, the mixture(s) may include a mixture of preformed vanadium pyrophosphate powder.

In another embodiment, the catalyst composition is a supported catalyst comprising a catalyst support in addition to the active phase element salts and optionally phosphorous and oxygen, in the amounts indicated above (wherein the molar ranges indicated are without regard to the moles of catalyst support, including any vanadium, bismuth, phosphorous or oxygen contained in the catalyst support). The total weight of the support (or modified support), based on the total weight of the catalyst, preferably is from 25 wt. % to 95 wt. %, e.g., from 40 wt. % to 70 wt. % or from 50 wt. % to 60 wt. %, and the total weight of the active phase is from 0.1 wt. % to 25 wt. %, based on the total weight of the catalyst composition. In a preferred embodiment, the weight of the active phase is at least 6 wt. % of the total catalyst composition weight.

The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, and mixtures thereof. Other supports may include, but are not limited to, iron oxide, magnesia, steatite, magnesium oxide, carbon, graphite, high surface area graphitized carbon, activated carbons, and mixtures thereof. Other supports may include coated structured forms such as coated metal foil, sintered metal forms and coated ceramic formed shapes such as shaped cordierite, platy alumina or acicular mullite forms. These listings of supports are merely exemplary and are not meant to limit the scope of the present invention.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt. % to 50 wt. %, e.g., from 0.2 wt. % to 25 wt. %, from 0.5 wt. % to 15 wt. %, or from 1 wt. % to 8 wt. %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

In some embodiments, the support may be a high surface area support, e.g., a support having a surface area of at least 1 m²/g, e.g., at least 20 m²/g or at least 50 m²/g, as determined by BET measurements. The catalyst support may include pores, optionally having an average pore diameter ranging from 5 nm to 200 nm, e.g., from 5 nm to 50 nm or from 10 nm to 25 nm. The catalyst optionally has an average pore volume of from 0.05 cm³/g to 3 cm³/g, e.g., from 0.05 cm³/g to 0.1 cm³/g or from 0.08 cm³/g to 0.1 cm³/g, as determined by BET measurements. Preferably, at least 50% of the pore volume or surface area, e.g., at least 70% or at least 80%, is provided by pores having the diameters discussed above. Pores may be formed and/or modified by pore modification agents, which are discussed below. In another embodiment, the ratio of microporosity to macroporosity ranges from 95:5 to 85:15, e.g., from 75:25 to 70:30. Microporosity refers to pores smaller than 2 nm in diameter, and movement in micropores may be described by activated diffusion. Mesoporosity refers to pores greater than 2 nm and less than 50 nm is diameter. Flow through mesopores may be described by Knudson diffusion. Macroporosity refers to pores greater than 50 nm in diameter and flow though macropores may be described by bulk diffusion. Thus, in some embodiments, it is desirable to balance the surface area, pore size distribution, catalyst or support particle size and shape, and rates of reaction with the rate of diffusion of the reactant and products in and out of the pores to optimize catalytic performance.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The aldol condensation catalysts described herein further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, methyl cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

In one embodiment, the catalyst compositions comprise a pore modification agent. A preferred type of pore modification agent is thermally stable and has a substantial vapor pressure at a temperature below 300° C., e.g., below 250° C. In one embodiment, the pore modification agent has a vapor pressure of at least 0.1 kPa, e.g., at least 0.5 kPa, at a temperature between about 150° C. and about 250° C., e.g., between about 150° C. and about 200° C.

In some embodiments, the pore modification agent has a relatively high melting point, e.g., greater than 60° C., e.g., greater than 75° C., so that it does not melt during compression of the catalyst precursor into a slug, tablet, or pellet. Preferably, the pore modification agent comprises a relatively pure material rather than a mixture. As such, lower melting components will not liquefy under compression during formation of slugs or tablets. For example, where the pore modification agent is a fatty acid, lower melting components of the fatty acid mixtures may be removed as liquids by pressing. If this phenomenon occurs during slug or tablet compression, the flow of liquid may disturb the pore structure and produce an undesirable distribution of pore volume as a function of pore diameter on the catalyst composition. In other embodiments, the pore modification agents have a significant vapor pressure at temperatures below their melting points, so that they can be removed by sublimination into a carrier gas.

For example, the pore modification agent may be a fatty acid corresponding to the formula CH₃(CH₂)ₓCOOH where x>8. Exemplary fatty acids include stearic acid (x=16), palmitic acid (x=14), lauric acid (x=10), myristic acid (x=12). The esters of these acids and amides or other functionalized forms of such acids, for example, stearamide (CH₃(CH₂)₁₆CONH₂) may also be used. Suitable esters may include methyl esters as well as glycerides such as stearin (glycerol tristearate). Mixtures of fatty acids may be used, but substantially pure acids, particularly stearic acid, are generally preferred over mixtures.

In addition, while fatty acids and fatty acid derivatives are generally preferred, other compositions which meet the functional requirements discussed above are also suitable for use as pore modification agents. Other preferred pore modification agents include but are not limited to polynuclear organic compounds such as naphthalene, graphite, natural burnout components such as cellulose and its cellulosic derivatives, starches, natural and synthetic oligomers and polymers such as polyvinyl alcohols and polyacrylic acids and esters.

Suitable reactors for configuration of reaction zone B may be those heat exchanger reactors already discussed with respect to reaction zone A.

The product gas mixture B which leaves reaction zone B and which comprises acrylic acid formed, unconverted acetic acid, at least one inert diluent gas other than steam, and steam, with or without (and optionally) molecular oxygen, can be separated in a manner known per se into the at least three streams X, Y and Z in a separation zone T.

For example, the separation can be achieved by fractional condensation, as recommended in documents DE-A 102007004960, DE-A 10 2007055086, DE-A 10243625, DE-A 10235847 and DE-A 19924532. In this procedure, the temperature of product gas mixture B is optionally first reduced by direct and/or indirect cooling, and product gas mixture B is then passed into a condensation column equipped with separating internals (for example mass transfer trays) and optionally provided with cooling circuits, and fractionally condensed ascending into itself within the condensation column. Appropriate selection of the number of theoretical plates in the condensation column allows streams X, Y and Z to be conducted out of the condensation column as separate fractions with the desired degree of enrichment in each case.

In some embodiments, stream X is generally removed with an acrylic acid content of ≧90% by weight, preferably ≧95% by weight. In the event of an increased purity requirement, stream X can, advantageously in application terms, be purified further by crystallization (preferably suspension crystallization) (see the aforementioned prior art documents and WO 01/77056). It will be appreciated that the stream X from the condensation column can also be purified further by rectification. It is possible in both ways, with a comparatively low level of complexity, to achieve acrylic acid purities of ≧99.9% by weight, which are suitable for production of water-absorbing resins by free-radical polymerization of monomer mixtures comprising acrylic acid and/or the sodium salt thereof.

The water-absorbing resins can be prepared, for example, as described in documents WO 2008/116840, DE-A 102005062929, DE-A 102004057874, DE-A 102004057868, DE-A 102004004496 and DE-A 19854575.

In a corresponding manner, stream Y is normally also obtained from the condensation column with an acetic acid content of ≧90% by weight, preferably ≧95% by weight. The stream Y thus removed can be recycled as such into reaction zone B to be used to prepare reaction gas input mixture B. It will be appreciated that it is also possible, prior to the recycling of the stream Y removed as described into reaction zone B, to further enrich the acetic acid content thereof by rectificative and/or crystallizative means (for example to acetic acid contents of ≧99% by weight), or to remove stream Y in the condensation column directly with such elevated purity by increasing the number of theoretical plates therein. Stream Z may exit the condensation column overhead.

Alternatively, it is also possible to proceed as recommended in documents DE-A 102009027401 and DE-A 10336386. After optional prior direct and/or indirect cooling, product gas mixture B in this procedure may be conducted using a countercurrent adsorption column with an organic solvent having a higher boiling point than acrylic acid at standard pressure (10⁵ Pa). Advantageously the column may be equipped with separating internals. Useful examples of the organic solvents are specified in DE-A 102009027401 and in DE-A 10336386. The acetic acid and acrylic acid present in product gas mixture B are absorbed into the organic solvent, while a stream Z leaves the adsorption column at the top thereof. From the absorbate comprising acetic acid and acrylic acid, it is possible to remove streams X and Y with the desired degree of enrichment in each case by rectification (fractional distillation) in a rectification column in a manner known per se through appropriate selection of the number of theoretical plates. In general, this degree of enrichment of acrylic acid or acetic acid will be at least 90% by weight, preferably at least 95% by weight. A subsequent crystallization purification of the stream X (for example as disclosed in WO 01/77056) may lead to acrylic acid purities of ≧99.9% by weight with a comparatively low level of complexity. These high purity streams may be suitable for production of water-absorbing resins, e.g., by free-radical polymerization of monomer mixtures comprising acrylic acid and/or the sodium salt thereof. The stream Y removed by rectification as described can be recycled, or after optional further crystallization- and/or rectification-based purification (for example to acetic acid contents of ≧99% by weight) into reaction zone B to obtain reaction gas input mixture B. By appropriately increasing the number of theoretical plates, it is also possible to remove stream Y from the absorbate by rectification directly with such a degree of enrichment.

In another embodiment, following the teaching of EP-A 551111 or EP-A 778255, it may also be possible to absorb the acrylic acid and acetic acid present in product gas mixture B into an aqueous absorbent in an absorption column. Stream Z may leaves the absorption column at the top thereof. Subsequent rectification of the aqueous absorbent, with optional inclusion of an azeotropic entraining agent, provides the desired streams X and Y.

The conversion of the acetic acid and acrylic acid present in reaction gas mixture B to the condensed phase to leave a gaseous stream Z can also be achieved, for example, by one-stage condensation of those constituents present in reaction gas mixture B whose boiling points at standard pressure are not above that of acetic acid. Subsequently, the condensate comprising acrylic acid and acetic acid can be separated again, in the degree of enrichment desired in each case, into at least one stream Y and at least one stream X.

Appropriately in some embodiments, in the process according to the invention, at least 90 mol %, preferably at least 95 mol %, more preferably at least 98 mol % or at least 99 mol % of the acetic acid present in product gas mixture B is recycled into reaction zone B to obtain reaction gas input mixture B. Possibly remove for CEL separation-related cases.

In one embodiment, acrylic acid polymerization may, for example, be achieved via solution polymerization or an aqueous emulsion polymerization or a suspension polymerization. In one embodiment, the acrylic acid present in stream X may be esterified with at least one alcohol having, for example, 1 to 8 carbon atoms (for example an alkanol such as methanol, ethanol, n-butanol, tert-butanol and 2-ethylhexanol) to give the corresponding acrylic esters (acrylate). The process for acrylic ester preparation may then again be followed by a process in which the acrylic ester prepared or a mixture of the acrylic ester prepared and one or more at least monoethylenically unsaturated monomers other than the acrylic ester prepared are polymerized to polymers (for example by free-radical means; the polymerization may, for example, be a solution polymerization or an aqueous emulsion polymerization or a suspension polymerization).

For the sake of good order, it should also be emphasized that deactivation of the different catalysts in the different reaction zones of the process according to the invention can be counteracted by correspondingly increasing the reaction temperature in the particular reaction zone (in order to keep the reactant conversion based on a single pass of the reaction gas mixture through the catalyst charge stable). It is also possible to regenerate the oxidic active materials of reaction zones A and B in a manner corresponding to that described for comparable oxidic catalysts in WO 2005/042459, by passing over an oxidizing oxygen-comprising gas at elevated temperature.

Reliable operation, especially in reaction zone A, can be ensured in the process according to the invention by an analogous application of the procedure described in WO 2004/007405.

The process according to the invention is advantageous for its broad and wide-ranging raw material basis in terms of time. In addition, the process, in contrast to the prior art processes, enables a smooth transition from “fossil acrylic acid” to “renewable acrylic acid” while maintaining the procedure.

“Fossil acrylic acid” is understood to mean acrylic acid for which the ratio of the molar amount of ¹⁴C atomic nuclei present in this acrylic acid to the molar amount of ¹²C atomic nuclei present in the same acrylic acid, n¹⁴C:n¹²C, is small.

“Renewable acrylic acid” is understood to mean acrylic acid for which the n¹⁴C:n¹²C ratio corresponds to V*, the ratio of n¹⁴C:n¹²C present in the CO₂ in the earth's atmosphere, the n¹⁴C:n¹²C ratio being determined by the procedure developed by Willard Frank Libby (http://de.wikipedia.orgn/wiki/Radikohlenstoffdatierung).

The terms “renewable carbon” and “fossil carbon” are used correspondingly in this document.

The process developed by Libby is based on the fact that, compared to the two carbon atom nuclei ¹²C and ¹³C, the third naturally occurring carbon nucleus ¹⁴C is unstable and is therefore also referred to as radiocarbon having a half-life of approximately 5700 years.

In the upper layers of the earth's atmosphere, ¹⁴C is constantly being newly formed by nuclei reaction. At the same time, ¹⁴C decomposes with a half-life of 5700 years by β-decomposition. An equilibrium forms in the earth's atmosphere between constant new formation and constant degradation, and so the proportion of the ¹⁴C nuclei in the carbon in the atmosphere on earth is constant over long periods; a stable ratio V* is present in the earth's atmosphere.

The radiocarbon produced in the atmosphere combines with atmospheric oxygen to give CO₂, which then gets into the biosphere as a result of photosynthesis. Since life forms (plants, animals, humans), in the course of their metabolism, constantly exchange carbon with the atmosphere surrounding them in this way, the same distribution ratio of the three carbon isotopes and hence the same n¹⁴C:n¹²C ratio is established in living organisms as is present in the surrounding atmosphere.

When this exchange is stopped at the time of death of the life form, the ratio between ¹⁴C and ¹²C in the dead organism changes because the decomposing ¹⁴C atomic nucleic are no longer replaced by new ones (the carbon present in the dead organism becomes fossil).

If the death of the organism (life form) was more than 50 000 years ago, the ¹⁴C content thereof is below the detection limit. Present and future biological (“renewable”) raw materials and chemicals produced therefrom have the particular current ¹⁴C concentration in the CO₂ in the atmosphere on the earth (this n¹⁴C:n¹²C ratio is represented by V*). Fossil carbon sources such as coal, mineral oil or natural gas, however, have already lain “dead” in the earth for several million years, just like chemicals produced therefrom, no longer comprise any ¹⁴C.

When fossil acetic acid (acetic acid obtained from fossil raw materials) and renewable formaldehyde (formaldehyde obtained from methanol obtained from renewable raw materials) are used in the process according to the invention, an acrylic acid is obtained whose n¹⁴C:n¹²C ratio is only approximately (⅓)×V.

When, in the process according to the invention, in contrast, acetic acid obtained from renewable raw materials and formaldehyde obtained from fossil methanol are used, an acrylic acid is obtained whose n¹⁴C:n¹²C ratio is approximately (⅔)×V.

When, in the process according to the invention, both fossil (renewable) acetic acid and fossil (renewable) formaldehyde are used, an acrylic acid is obtained whose n¹⁴C:n¹²C ratio is essentially zero.

When the possibility of blending renewable and fossil starting materials (raw materials) is additionally considered in the process according to the invention, the manufacturer of acrylic acid, when employing the inventive procedure, is able to adjust the “renewable level” of the acrylic acid to be supplied to this customer (the n¹⁴C:n¹²C ratio desired by the customer for the acrylic acid to be supplied) without altering the preparation process, e.g., with one and the same production plant.

By esterifying an acrylic acid for which V=V* with biomethanol or bioethanol, it is possible to obtain acrylic esters whose n¹⁴C to n¹²C ratio is likewise V*.

A further advantage of the inventive procedure is that the target product of reaction zone A does not require removal from product gas mixture A in order to be able to be employed for production of reaction gas input mixture B. This ensures both high economy and an efficient energy balance for the process according to the invention. Furthermore, in the case of condensation of acetic acid with formaldehyde, neither glyoxal nor propionic acid is formed as a by-product, as is necessarily the case for a heterogeneously catalyzed partial oxidation of propylene, propane, acrolein, propionaldehyde and/or glycerol to acrylic acid (see WO 2010/074177).

Furthermore, the process according to the invention ensures a high space-time yield coupled with simultaneously high target product selectivity based on the reactants converted.

### EXAMPLES

### Example 1

Catalyst compositions were prepared using a bismuth salt Bi(NO₃)₃ hydrate and a vanadium precursor, e.g., NH₄VO₃. Colloidal silica, deionized water, and ethylene glycol were combined and mixed. An organic acid, e.g. citric acid, was added to the mixture and the mixture was heated to 50° C. A calculated amount of NH₄VO₃ was added to the mixture and the resulting solution was heated to 80° C. with stirring. An amount of a bismuth nitrate was added to the heated mixture. A 2 wt % solution of methyl cellulose was added to the bismuth salt/vanadium salt/vanadium precursor solution and stirred at 80 C. A calculated amount of phosphoric acid (85%) was added and the resulting solution was stirred. The final mixture was then evaporated to dryness overnight in a drying oven at 120° C., ground and calcined using the following temperature profile:
i) heating from room temperature to 160° C. at a rate of 10° C. per minute;
ii) heating at 160° C. for 2 hours;
iii) heating from 160° C. to 250° C. at a rate of 3° C. per minute;
iv) heating at 250° C. for 2 hours;
v) heating from 250° C. to 300° C. at a rate of 3° C. per minute;
vi) heating at 300° C. for 6 hours;
vii) heating from 300° C. to 450° C. at a rate of 3° C. per minute; and
viii) heating at 450° C. for 6 hours.

### Example 2

Approximately 100 mL of isobutanol was heated to 90° C. in a flask with a mechanical stirrer and condenser. The desired amount of V₂O₅ (11.32 g) was slowly added as a powder to the well stirred hot isobutanol. Once the V₂O₅ was added, 85% H₃PO₄ (8.2 g) was slowly added to the hot mixture with agitation. Once the addition of H₃PO₄ was complete, the temperature of the mixture was increased to 100-108° C. and the mixture was stirred at this temperature for about 14 hours.

Bismuth nitrate hydrate (30.2 g) is dissolved in 10% HNO₃. BiPO₄ was formed and precipitated by the slow addition of diluted H₃PO₄ (8.2 g-85% H₃PO₄) with constant stirring. The mixture was stirred for 1 hour and then the BiPO₄ was collected via filtration or centrifugation. The solid BiPO₄ is washed with deionized water three times.

The BiPO₄ was added to the V₂O₅—H₃PO₄-iBuOH mixture and the mixture was stirred and refluxed for one hour. The mixture was allowed to cool and the catalyst in solid form was isolated via filtration or centrifugation. The solid was washed once with EtOH and twice more with deionized water. The solid was dried overnight at 120° C. with flowing air. The dried solid was ground to powder and mixed together and then calcined using the temperature profile of Example 1.

### Example 3

Colloidal silica (1.3 g), citric acid (25.7 g), ethylene glycol (18.5 g), deionized water (12 g) were combined and heated to 50° C. with stirring. NH₄VO₃ (13.6 g) was added as a fine powder to the citric acid mixture and the resulting solution was heated to 80° C. and stirred for 30 minutes. Bismuth nitrate hydrate (0.9 g) was dissolved in 10% HNO₃ solution and slowly added to the vanadyl solution. The mixture was stirred at 80° C. for 30 minutes.

The mixture was cooled and a 2 wt % solution of methyl cellulose (100 g) was added to the mixture and stirred for 30 minutes. Then 85% H₃PO₄ solution (15.7 g) was slowly added to the mixture and the resulting solution was stirred for 30 minutes. The final mixture was heated overnight in a drying oven at 120° C. at which time the final mixture underwent thermogellation, which resulted in a porous foam-like material. The resulting foam-like material was ground to mix and calcined using the temperature profile of Example 1.

Table 5 shows surface area, pore volume, and pore size of various catalyst compositions comprising vanadium/bismuth prepared via the methods of Examples 1-3. A bismuth free vanadium catalyst is also provided as comparative example.

TABLE 5
Catalyst Compositions

| | | | BET | BET | BET |
|---|---|---|---|---|---|
| | | | Surface | Ave. Pore | Ave. |
| | Catalyst | | Area | Vol. | Pore Size |
| Catalyst | Formula | Preparation Method | (m²/g) | (cm³) | (nm) |
| 1 | V₁₀Bi_{0.16}P_{11.7}O₅₁ | Example 1 | 11 | 0.032 | 12 |
| | | 2.5% SiO₂, 10% methylcellulose | | | |
| 2 | V₁₀Bi₅P_{17.25}O₇₀ | Example 2 | 10 | 0.024 | 10 |
| | | 10 mole V₂O₅, 5 mole BiNO₃ | | | |
| 3 | V₁₀Bi_{0.16}P₁₀O₄₆ | Example 3 | 9 | 0.023 | 10 |
| | | 0.16 mole BiPO₄, 10 mole VOPO₄ | | | |
| Comp. A | VPO | Commercial VPO | n/a | n/a | n/a |
| Comp. B | VP_{1.15}O | NH₄VO₃, citric acid, 5% SiO₂, 10% | 8.4 | 0.025 | 11.9 |
| | | methyl cellulose | | | |

The effect of Bi doping was studied. The results are presented in Table 5. Catalysts 1 and 3 have a V:Bi ratio of 62.5:1 and Catalyst 2 has a V:Bi ratio of 2:1. Catalyst Comp. A and Catalyst Comp. B are commercially available bismuth free vanadium catalysts. As shown in Table 5, an increase of V:Bi ratio from 2:1 to 63:1 does not appear to significantly affect the surface area, pore volume, and/or pore size of the resultant catalysts.

### Example 4

A reaction feed comprising acetic acid, formaldehyde, methanol, water, oxygen, and nitrogen was passed through a fixed bed reactor comprising the catalysts shown in Table 6. The formaldehyde (and the formaldehyde utilized in all of the examples, unless stated otherwise) was formed in a formox unit. The reactions were conducted at a reactor temperature of 375° C. and a GHSV of 2000 Hr⁻¹. Acrylic acid and methyl acrylate (collectively, “acrylate product”) were produced. The conversions, selectivities, and space time yields are shown in Table 6.

TABLE 6
Acrylate Production

| | | HOAc | Acrylate | | |
|---|---|---|---|---|---|
| | Runtime | Conv. | Selectivity | Acrylate | Acrylate STY |
| Catalyst | (h) | (%) | (%) | Yield (%) | (g/hr/L) |
| 1 | 0.6 | 41 | 80 | 33 | 437 |
| | 0.8 | 42 | 78 | 33 | 435 |
| | 1.6 | 41 | 81 | 33 | 433 |
| | 2.4 | 41 | 81 | 33 | 438 |
| | 2.8 | 41 | 81 | 33 | 430 |
| | 3.7 | 41 | 81 | 33 | 439 |
| | 4.2 | 41 | 80 | 33 | 432 |
| | 5.0 | 41 | 81 | 33 | 435 |
| 2 | 0.8 | 40 | 80 | 32 | 422 |
| | 2.4 | 40 | 81 | 32 | 426 |
| | 3.7 | 40 | 80 | 32 | 420 |
| | 5.0 | 39 | 81 | 32 | 417 |
| | 6.2 | 40 | 80 | 32 | 416 |
| | 24.0 | 43 | 79 | 34 | 447 |
| | 25.2 | 39 | 82 | 32 | 417 |
| | 26.5 | 39 | 81 | 32 | 415 |
| | 27.7 | 41 | 80 | 33 | 432 |
| 3 | 1.7 | 38 | 77 | 29 | 381 |
| | 2.9 | 39 | 77 | 30 | 391 |
| | 4.0 | 39 | 77 | 30 | 392 |
| | 5.3 | 39 | 77 | 30 | 393 |
| | 6.1 | 39 | 78 | 30 | 395 |
| | 24.9 | 38 | 79 | 30 | 390 |
| | 26.0 | 38 | 79 | 30 | 395 |
| | 27.2 | 38 | 79 | 30 | 393 |
| Comp. A | 0.8 | 27 | 85 | 23 | 304 |
| | 1.7 | 24 | 94 | 22 | 289 |
| | 2.7 | 23 | 95 | 22 | 280 |
| | 3.9 | 22 | 97 | 21 | 277 |
| Comp. B | 1.2 | 39 | 77 | 30 | 391 |
| | 2.3 | 38 | 76 | 29 | 378 |
| | 3.3 | 37 | 77 | 28 | 369 |
| | 18.0 | 34 | 73 | 24 | 319 |
| | 19.3 | 33 | 72 | 24 | 310 |
| | 20.4 | 31 | 79 | 24 | 315 |
| | 21.4 | 31 | 78 | 24 | 310 |
| | 22.5 | 30 | 78 | 24 | 309 |

Acetic acid conversion, acrylate selectivity, acrylate yield, and acrylate space time yield were measured for Catalysts 1-3, Comp. A, and Comp. B at various time points of the reaction. As shown, Catalysts 1-3 maintained steady acetic acid conversion, acrylate selectivity, acrylate yield, and acrylate space time yield over shorter time periods, i.e., Catalysts 1-3 showed little if any catalyst deactivation over shorter time periods, e.g., 5.3 hours or less. In comparison, acetic acid conversion, acrylate yield, and acrylate space time yield for Comp. A and Comp. B decreased after only 3.9 hours and 3.3 hours, respectively. Also, over longer time periods, e.g., for approximately 28 hours, Catalysts 2 and 3 continued to demonstrate maintenance of steady reaction performance, as compared to Comp. B, which deactivated significantly over 22.5 hours. For example, for Comp. B, the acetic acid conversion dropped from 39% to 30%, acrylate yield dropped from 30% to 24%, and the space time yield dropped from 391 g/hr/L to 309 g/hr/L. In addition, the data suggests that even with a small amount of bismuth, the bismuth containing vanadium catalysts provides surprising and unexpected stability over longer time periods. In sum, Catalysts 1-3 surprisingly and unexpectedly demonstrate significant improvements in both short term and long term catalyst deactivation (as measured in acetic acid conversion, acrylate selectivity, acrylate yield, and/or STY) as compared to Comp. A and/or Comp. B.

In addition to the deactivation improvements, Catalysts 1-3, all of which contain bismuth, unexpectedly outperform Comp. A and Comp. B, which are conventional bismuth-free commercially available vanadium catalysts. For example, Catalysts 1-3 demonstrate average acetic acid conversions of 41%, 40%, and 39%, respectively, while Comp. A and Comp. B demonstrate an average acetic acid conversion of only 24% and 34%, respectively. Also, Catalysts 1-3 demonstrate average acrylate STY of 435 g/hr/L, 423 g/hr/L, and 391 g/hr/L, while Comp. A and Comp. B demonstrate average yields of only 288 g/hr/L and 338 g/hr/L. In addition, Catalysts 1-3 demonstrate average acrylate yields of 33%, 32%, and 30%, while Comp. A and Comp. B demonstrate an average yield of only 22% and 26%, respectively. Catalysts 1 and 3 have a V:Bi ratio of 62.5:1. As shown in Table 3, even at a low level, bismuth surprisingly and unexpectedly increases the acetic acid conversion, acrylate selectivity, acrylate yield and/or acrylate space time yield.

Table 7 shows surface area, pore volume, and pore size of an additional catalyst composition comprising vanadium and bismuth prepared via the preparation method of Example 2. Comp. B is also shown as a comparative example.

TABLE 7
Catalyst Compositions

| | | | | BET | BET |
|---|---|---|---|---|---|
| | | | BET | Ave. | Ave. |
| | | | Surface | Pore | Pore |
| | Catalyst | Preparation | Area | Vol. | Size |
| Catalyst | Formula | Methods | (m²/g) | (cm³) | (nm) |
| 4 | V₁₀Bi_{0.16}P_{11.7}O₅₁ | Example 2 | 16.3 | 0.042 | 10.3 |
| | | 10 mole V₂O₅, | | | |
| | | 0.16 mole BiNO₃ | | | |
| Comp. B | VP_{1.15}O | NH₄VO₃, citric | 8.4 | 0.025 | 11.9 |
| | | acid, 5% SiO₂, | | | |
| | | 10% methyl | | | |
| | | cellulose | | | |

Catalyst 4 was prepared as described above. Catalyst Comp. B is a bismuth free vanadium catalyst prepared by reducing ammonium metavanadate with citric acid then adding 5.0 wt % SiO₂ and 10% methylcellulose. As shown in Table 4, Catalyst 4 has similar surface area, pore volume, and smaller pore size as the bismuth free vanadium catalyst.

### Example 5

A reaction feed comprising acetic acid, formaldehyde, methanol, water, oxygen, and nitrogen was passed through a fixed bed reactor comprising the catalysts shown in Table 4 above. The reactions were conducted at a reactor temperature of 375° C. and a GHSV at 2000 Hr⁻¹. Acrylic acid and methyl acrylate (collectively, “acrylates”) were produced. The conversions, selectivities, and space time yields are shown in Table 8.

TABLE 8
Acrylate Production

| | | HOAc | Acrylate | | |
|---|---|---|---|---|---|
| | Runtime | Conv. | Selectivity | Acrylate | Acrylate STY |
| Catalyst | (h) | (%) | (%) | Yield (%) | (g/hr/L) |
| 4 | 1.8 | 31 | 84 | 26 | 342 |
| | 2.9 | 31 | 82 | 26 | 335 |
| | 4.2 | 32 | 81 | 26 | 338 |
| | 17.6 | 32 | 82 | 26 | 342 |
| | 18.9 | 33 | 81 | 27 | 347 |
| | 20.2 | 33 | 81 | 27 | 349 |
| | 23.4 | 33 | 81 | 27 | 351 |
| Comp. B | 1.2 | 39 | 77 | 30 | 391 |
| | 2.3 | 38 | 76 | 29 | 378 |
| | 3.3 | 37 | 77 | 28 | 369 |
| | 18.0 | 34 | 73 | 24 | 319 |
| | 19.3 | 33 | 72 | 24 | 310 |
| | 20.4 | 31 | 79 | 24 | 315 |
| | 21.4 | 31 | 78 | 24 | 310 |
| | 22.5 | 30 | 78 | 24 | 309 |

Acetic acid conversion, acrylate selectivity, acrylate yield and acrylate space time yield were measured for Catalyst 4 and Comp. B various time points of the reaction. As shown, Catalyst 4 maintained steady acetic acid conversion, acrylate selectivity, acrylate yield, and acrylate space time yield over both long and short time periods. In comparison, when Comp. B was utilized, the acetic acid conversion, acrylate yield, and acrylate space time yield significantly decreased over the course of 22.5 hours. For example, as discussed above, the acetic acid conversion dropped from 39% to 30% over 22.5 hours, acrylate yield dropped from 30% to 24%, and the space time yield dropped from 391 g/hr/L to 309 g/hr/L.

### Example 6

Multiple catalyst compositions were prepared using the following general procedure and stoichiometric amounts of the indicated starting materials. Titanium isopropoxide in isopropanol was added to an aqueous mixture of colloidal silica and stirred for 30 minutes to form a titania suspension. Separately, citric acid was dissolved in a mixture of ethylene glycol and deionized H₂O. The solution was heated to about 50° C. with stirring. Next, the calculated amounts of NH₄VO₃ was added to the citric acid mixture and the resulting vanadyl solution was then heated to 80° C. with stirring and kept at this temperature for 60 minutes. A 2 wt. % solution of methyl cellulose was added to the vanadyl solution and the resulting mixture was stirred for 15 minutes at 80° C. Bismuth nitrate was dissolved in 10% HNO₃ and the resultant solution was added to the vanadyl-methylcellulose mixture and stirred at 80° C. to allow the vanadyl-methylcellulose-bismuth solution to cool. The cooled mixture was slowly added to the titania suspension. The resulting mixture was stirred for 15 minutes at room temperature. H₃PO₄ was slowly added to the mixture and the resulting mixture was vigorously mixed at room temperature. The final mixture was dried (120° C.) overnight/air and calcined using the following temperature program:
(1) heating to 160° C. at a rate of 10° C. per minute, and holding at 160° C. for 2 hours;
(2) heating to 250° C. at a rate of 3° C. per minute, and holding at 250° C. for 2 hours;
(3) heating to 300° C. at a rate of 3° C. per minute, and holding at 300° C. for 6 hours; and
(4) heating to 450° C. at a rate of 3° C. per minute, and holding at 450° C. for 6 hours.

Table 9 lists Catalysts 1-9, each of which are VBiTi catalysts prepared via the preparation method of Example 6.

TABLE 9
Catalyst Compositions

| Catalyst | Catalyst Formula | Preparation Details |
|---|---|---|
| 1 | V₁₀Bi_{1.0}Ti₄P_{21.2}O₈₄ | citric acid, 5.8% SiO₂, |
| | | 10% methylcellulose |
| 2 | V₁₀Bi_{1.0}Ti_{0.16}P_{12.9}O₅₅ | citric acid, 5.8% SiO₂, |
| | | 10% methylcellulose |
| 3 | V₁₀Bi_{0.16}Ti₄P_{20.3}O₈₁ | citric acid, 5.8% SiO₂, |
| | | 10% methylcellulose |
| 4 | V₁₀Bi_{0.16}Ti_{0.16}P_{12.0}O₅₂ | citric acid, 5.8% SiO₂, |
| | | 10% methylcellulose |
| 5 | V₁₀Bi_{0.16}Ti₁₀P_{33.2}O₁₂₆ | citric acid, 5.8% SiO₂, |
| | | 10% methylcellulose |
| 6 | V₁₀Bi₁₀Ti₁₀P₄₄O₁₆₄ | citric acid, 5.8% SiO₂, |
| | | 10% methylcellulose |
| 7 | V₆Bi_{0.16}Ti₄P_{15.7}O₆₁ | citric acid, 5.8% SiO₂, |
| | | 10% methylcellulose |
| 8 | V₁₀Bi₁₀Ti₄P_{31.1}O₁₁₉ | citric acid, 5.8% SiO₂, |
| | | 10% methylcellulose |
| Comp. A | Commercial VPO | Commercial |
| Comp. B | VPO | citric acid, 5.0% ethylene glycol, |
| | | 10% methylcellulose |
| Comp. C | V₂Bi₁₀Ti₁₀P_{34.8}O₁₂₄ | citric acid, 5.8% SiO2, |
| | | 10% methylcellulose |

Catalysts 1-8 and Comp. C were made using citric acid, 5.8% SiO₂, and 10% methylcellulose. Catalyst 1 has a V:Bi:Ti ratio of 10:1.0:4. Catalyst 2 has a V:Bi:Ti ratio of 10:1.0:0.16. Catalyst 3 has a V:Bi:Ti ratio of 10:0.16:4. Catalyst 4 has a V:Bi:Ti ratio of 10:0.16:0.16. Catalyst 5 has a V:Bi:Ti ratio of 10:0.16:10. Catalyst 6 has a V:Bi:Ti ratio of 10:10:10. Catalyst 7 has a V:Bi:Ti ratio of 6:0.16:4. Catalyst 8 has a V:Bi:Ti ratio of 10:10:4. Comp. C has a V:Bi:Ti ratio of 2:10:10.

### Example 7

A reaction feed comprising acetic acid, formaldehyde, methanol, water, oxygen, and nitrogen was passed through a fixed bed reactor comprising Catalysts 1-8 and Comp. C shown in Table 10. The reactions for Catalysts 1-8 and Comp. C were conducted at a reactor temperature of 375° C., a GHSV of 2000 Hr⁻¹, total organics of 32 mole %, acetic acid to formaldehyde ratio of 1.5, oxygen feed concentration of 4.8%, water feed concentration of 7.2 mole %, nitrogen feed concentration of 56 mole %. Acrylic acid and methyl acrylate (collectively, “acrylate product”) were produced. Acetic acid conversion, acrylate selectivity, acrylate yield, and acrylate space time yield were measured for Catalysts 1-9 at various time points of the reaction. Commercial VPO catalysts, Comp. A and Comp. B, were also tested under the same conditions. The results are shown in Table 10.

TABLE 10
Acrylate Production

| | | HOAc | Acrylate | | |
|---|---|---|---|---|---|
| | Runtime | Conv. | Selectivity | Acrylate | Acrylate STY |
| Catalyst | (h) | (%) | (%) | Yield (%) | (g/hr/L) |
| 1 | 0.6 | 43 | 80 | 35 | 457 |
| | 1.5 | 43 | 81 | 35 | 461 |
| | 2.5 | 43 | 81 | 35 | 464 |
| | 19.2 | 45 | 80 | 36 | 477 |
| | 20.5 | 46 | 81 | 37 | 487 |
| | Avg. | 44 | 81 | 35 | 469 |
| 2 | 0.8 | 35 | 83 | 29 | 388 |
| | 2.0 | 35 | 84 | 29 | 387 |
| | 3.2 | 35 | 84 | 29 | 386 |
| | 4.5 | 34 | 84 | 29 | 382 |
| | 6.6 | 35 | 83 | 29 | 383 |
| | 8.0 | 35 | 83 | 29 | 385 |
| | 8.9 | 35 | 83 | 29 | 381 |
| | 9.8 | 35 | 84 | 29 | 383 |
| | 25.5 | 34 | 84 | 29 | 377 |
| | 26.5 | 34 | 83 | 28 | 374 |
| | 28.0 | 34 | 83 | 29 | 377 |
| | Avg. | 35 | 83 | 29 | 382 |
| 3 | 0.8 | 32 | 82 | 26 | 342 |
| | 2.0 | 32 | 82 | 26 | 346 |
| | 3.2 | 32 | 82 | 26 | 346 |
| | 4.5 | 32 | 82 | 26 | 351 |
| | 6.6 | 33 | 82 | 27 | 354 |
| | 8.0 | 32 | 81 | 26 | 349 |
| | 8.9 | 32 | 82 | 27 | 351 |
| | 9.8 | 33 | 80 | 27 | 352 |
| | 25.5 | 33 | 81 | 27 | 355 |
| | 26.5 | 32 | 81 | 26 | 350 |
| | 28.0 | 33 | 81 | 27 | 359 |
| | Avg. | 32 | 82 | 26 | 350 |
| 4 | 0.5 | 29 | 80 | 23 | 311 |
| | 1.4 | 31 | 83 | 26 | 346 |
| | 2.4 | 32 | 82 | 26 | 344 |
| | 19.9 | 32 | 82 | 26 | 343 |
| | 22.3 | 31 | 82 | 26 | 341 |
| | 23.3 | 32 | 80 | 26 | 339 |
| | 24.8 | 31 | 81 | 25 | 338 |
| | Avg. | 31 | 81 | 25 | 337 |
| 5 | 0.5 | 23 | 85 | 20 | 257 |
| | 1.4 | 25 | 85 | 21 | 274 |
| | 2.4 | 23 | 96 | 22 | 286 |
| | 19.9 | 29 | 84 | 24 | 315 |
| | 22.3 | 29 | 85 | 25 | 325 |
| | 23.3 | 30 | 82 | 25 | 322 |
| | 24.8 | 31 | 81 | 25 | 330 |
| | Avg. | 27 | 85 | 23 | 302 |
| 6 | 0.7 | 29 | 91 | 26 | 343 |
| | 2.0 | 30 | 90 | 27 | 354 |
| | 3.3 | 30 | 90 | 27 | 358 |
| | 4.5 | 31 | 89 | 27 | 362 |
| | 20.2 | 34 | 89 | 30 | 395 |
| | 21.6 | 34 | 89 | 30 | 403 |
| | 22.4 | 34 | 90 | 31 | 407 |
| | Avg. | 32 | 90 | 28 | 375 |
| 7 | 0.5 | 28 | 89 | 25 | 334 |
| | 1.5 | 30 | 88 | 26 | 343 |
| | 2.3 | 30 | 88 | 26 | 347 |
| | 15.8 | 31 | 86 | 27 | 353 |
| | 16.8 | 31 | 87 | 27 | 354 |
| | 18.3 | 31 | 88 | 27 | 364 |
| | Avg. | 30 | 88 | 26 | 349 |
| 8 | 0.5 | 39 | 81 | 32 | 412 |
| | 1.5 | 40 | 81 | 32 | 416 |
| | 2.3 | 40 | 81 | 32 | 418 |
| | 15.8 | 39 | 80 | 31 | 411 |
| | 16.8 | 39 | 80 | 31 | 407 |
| | 18.3 | 39 | 80 | 31 | 405 |
| | Avg. | 39 | 80 | 31 | 412 |
| VPO | 0.8 | 27 | 85 | 23 | 304 |
| Comp. A | 1.7 | 24 | 94 | 22 | 289 |
| | 2.7 | 23 | 95 | 22 | 280 |
| | 3.9 | 22 | 97 | 21 | 277 |
| | Avg. | 24 | 93 | 22 | 287 |
| VPO | 0.8 | 22 | 90 | 20 | 255 |
| Comp. B | 1.7 | 22 | 90 | 19 | 253 |
| | 2.7 | 22 | 87 | 19 | 251 |
| | 3.9 | 22 | 90 | 19 | 254 |
| | Avg. | 22 | 89 | 19 | 254 |
| Comp. C | 0.7 | 13 | 88 | 11 | 146 |
| | 2.0 | 12 | 83 | 10 | 126 |
| | 3.3 | 11 | 84 | 10 | 126 |
| | 4.5 | 12 | 84 | 10 | 127 |
| | Avg. | 12 | 85 | 10 | 131 |

Catalysts 1-8, all of which contain vanadium, bismuth and titanium, in the amounts discussed herein, unexpectedly outperform Comp. A and Comp. B, which are conventional bismuth-free and titanium-free commercially available catalysts, and Comp. C, which has a lower vanadium:bismuth molar ratio and a lower vanadium:titanium molar ratio than Catalysts 1-8. For example, Catalysts 1-8 demonstrate average acetic acid conversions of 44%, 35%, 32%, 31%, 27%, 32%, 30% and 39%, respectively, while Comp. A and Comp. B demonstrate an average acetic acid conversion of only 24% and 22%, respectively. Also, Catalysts 1-8 demonstrate average acrylate STY of 469 g/hr/L, 382 g/hr/L, 350 g/hr/L, 337 g/hr/L, 302 g/hr/L, 375 g/hr/L, 349 g/hr/L and 412 g/hr/L, respectively, while Comp. A and Comp. B demonstrate an average STY of only 287 g/hr/L and 254 g/hr/L, respectively. In addition, Catalysts 1-8 demonstrate average acrylate yields of 35%, 29%, 26%, 25%, 23%, 28%, 26% and 31%, respectively, while Comp. A, Comp. B, and Comp. C demonstrate an average yield of only 22%, 19%, and 10%, respectively.

Surprisingly and unexpectedly, as shown, the catalysts of the present invention maintained steady or increase acetic acid conversion, acrylate selectivity, acrylate yield, and acrylate space time yield over long time periods, e.g., Catalysts 1-8 showed little if any catalyst deactivation. For example, over a 20.5 hour period, the acetic acid conversion for Catalyst 1 remains between 43% and 46%. Over a 28.0 hour period, the acetic acid conversion for Catalyst 2 remains between 34% and 35%. Similarly, over a 28.0 hour period, the acetic acid conversion for Catalyst 3 remains between 32% and 33%. In addition, over a 24.8 hour period, the acetic acid conversion for Catalyst 4 remains between 29% and 32%. Similarly, over a 24.8 hour period, the acetic acid conversion for Catalyst 5 remains between 23% and 31%. Over a 22.4 hour period, the acetic acid conversion for Catalyst 6 remains between 29% and 34%. Over an 18.3 hour period, the acetic acid conversion for Catalyst 7 remains between 28% and 31%. Lastly, over a 18.3 hour period, the acetic acid conversion for Catalyst 8 remains between 39% and 40%. In comparison, acetic acid conversion for Comp. A decreases significantly from 27% to 22% after only 3.9 hours.

As shown in Table 10, Catalysts 1-8 have steady acrylate yield and acrylate space time yield. For example, Catalyst 1 has an acrylate yield between 35% and 37% and acrylate space time yield between 457 g/hr/L and 487 g/hr/L. Catalyst 2 has an acrylate yield between 28% and 29% and acrylate space time yield between 374 g/hr/L and 388 g/hr/L. Catalyst 3 has an acrylate yield between 26% to 27% and acrylate space time yield between 342 g/hr/L and 359 g/hr/L. Catalyst 4 has an acrylate yield between 23% to 26% and acrylate space time yield between 311 g/hr/L and 346 g/hr/L. Catalyst 5 has an acrylate yield between 20% and 25% and acrylate space time yield between 257 g/hr/L and 320 g/hr/L. Catalyst 6 has an acrylate yield between 26% and 31% and acrylate space time yield between 343 g/hr/L and 407 g/hr/L. Catalyst 7 has an acrylate yield between 25% and 27% and acrylate space time yield between 334 g/hr/L and 364 g/hr/L. Catalyst 8 has an acrylate yield between 31% and 32% and acrylate space time yield between 405 g/hr/L and 412 g/hr/L.

Surprisingly and unexpectedly, even at lower amounts of bismuth and titanium, the catalyst of the present invention provides better acetic acid conversion, acrylate yield, and acrylate STY than the titanium-free and bismuth-free commercially available catalysts. For example, Catalyst 4 with the formula V₁₀Bi_{0.16}Ti_{0.16}P_{12.0}O₅₂ has an average acetic acid conversion of 31%, an average acrylate yield of 25%, and an average acrylate STY of 337 g/hr/L. In comparison, Comp. A and Comp. B have an average acetic acid conversion of 24% and 22%, respectively, an average acrylate yield of 22% and 19%, respectively, and an average acrylate STY of 287 g/hr/L and 254 g/hr/L, respectively. Thus, even though relatively small amounts of bismuth and titanium are utilized, significant improvements in catalyst performance are demonstrated, as compared to conventional catalyst compositions.

Surprisingly and unexpectedly, the amount of vanadium in the catalyst affects the acetic acid conversion, acrylate yield and acrylate STY. For example, Catalysts 1-8 have molar ratio of V:Ti and V:Bi greater than 0.2:1 and each of these catalysts has a high acetic acid conversion, acrylate yield, acrylate selectivity and acrylate STY. In comparison, Comp. C has the formula V₂Bi₁₀Ti₁₀P_{34.8}O₁₂₄, where the ratios of V:Ti and V:Bi are lower than those of the present invention, e.g., 0.2:1. Comp. C has acetic acid conversion between 13% and 11%, acrylate selectivity between 33% and 83%, acrylate yield between 11% and 10%, and an acrylate STY between 146 g/hr/L and 126 g/hr/L, which are significantly lower than the results achieved using Catalysts 1-8.

As shown by the data, Catalysts 1-8, which comprise vanadium, bismuth, and titanium at the specific molar ratios discussed herein, outperform commercially available VPO catalyst Comp. A and Comp. B, as well as Comp. C, which comprises bismuth and titanium, but has a lower vanadium:bismuth molar ratio and a lower vanadium:titanium molar ratio than Catalysts 1-8.

### Example 8

Catalyst compositions were prepared using a bismuth salt, a tungsten salt, and a vanadium precursor, e.g., NH₄VO₃. Colloidal silica, deionized water, and ethylene glycol were combined and mixed. An organic acid, e.g., oxalic acid or citric acid, was added to the mixture and the mixture was heated to 50° C. A calculated amount of NH₄VO₃ was added to the mixture and the resulting solution was heated to 80° C. with stirring. Bismuth nitrate and ammonium metatungstate were added to the heated mixture. A 2 wt % solution of methyl cellulose was added to the bismuth salt/tungsten salt/vanadium precursor solution and stirred at 80° C. A calculated amount of phosphoric acid (85%) was added and the resulting solution was stirred. The final mixture was then evaporated to dryness in a 120° C. drying oven overnight. The resulting solid was calcined using the following temperature profile:
i) heating from room temperature to 160° C. at a rate of 10° C. per minute;
ii) heating at 160° C. for 2 hours;
iii) heating from 160° C. to 250° C. at a rate of 3° C. per minute;
iv) heating at 250° C. for 2 hours;
v) heating from 250° C. to 300° C. at a rate of 3° C. per minute;
vi) heating at 300° C. for 6 hours;
vii) heating from 300° C. to 450° C. at a rate of 3° C. per minute; and
viii) heating at 450° C. for 6 hours.

### Example 9

Approximately 100 mL of isobutanol was heated to 90° C. in a flask with a mechanical stirrer and condenser. The desired amount of V₂O₅ (11.32 g) was slowly added as a powder to the well stirred hot isobutanol. Once the V₂O₅ was added, 85% H₃PO₄ (8.2 g) was slowly added with agitation to the hot mixture. Once the addition of H₃PO₄ was complete, the temperature of the mixture was increased to 100-108° C. and the mixture was stirred at this temperature for about 14 hours.

Bismuth nitrate hydrate (30.2 g) is dissolved in 10% HNO₃. BiPO₄ was formed and precipitated by the slow addition of diluted H₃PO₄ (8.2 g-85% H₃PO₄) with constant stirring. The mixture was stirred for 1 hour and then the BiPO₄ was collected via filtration or centrifugation. The solid BiPO₄ is washed with deionized water three times.

The BiPO₄ was added to the V₂O₅—H₃PO₄-iBuOH mixture and the mixture was stirred at reflux for one hour. The mixture was allowed to cool and the catalyst in solid form was isolated via filtration or centrifugation. The solid was washed once with EtOH and twice more with deionized water. The solid was dried overnight at 120° C. with flowing air. The ammonium metatungstate was dissolved in water and added to the VBiPO solid and dried. The final VBiWPO solid was ground to form a mixture and then calcined using the temperature profile of Example 8.

### Example 10

Colloidal silica (1.3 g), citric acid (25.7 g), ethylene glycol (18.5 g), deionized water (12 g) were combined and heated to 50° C. with stirring. NH₄VO₃ (13.6 g) was added as a fine powder to the citric acid mixture and the resulting solution was heated to 80° C. and stirred for 30 minutes. The ammonium megatungstate was dissolved in water and the solution was added to the vanadyl solution and stirred for 15 minutes at 80° C. Bismuth nitrate hydrate (0.9 g) was dissolved in 10% HNO₃ solution and slowly added to the vanadyl solution. The mixture was stirred at 80° C. temperature for 30 minutes.

The mixture was cooled and a 2 wt % solution of methyl cellulose (100 g) was added to the mixture and stirred for 30 minutes. Then 85% H₃PO₄ solution (15.7 g) was slowly added to the mixture and the resulting solution was stirred for 30 minutes. The final mixture was heated overnight in a drying oven at 120° C. at which time the final mixture underwent thermogellation, which resulted in a porous foam-like material. The resulting foam-like material was ground to mix and calcined using the temperature profile of Example 8.

Table 11 shows surface area, pore volume, and pore size of catalyst compositions comprising vanadium/bismuth/tungsten prepared via the method of Example 8. The effect of vanadium, tungsten and/or bismuth (as well as other preparation conditions) on the surface area, pore volume, and pore size of the resultant catalyst was studied. Catalyst 1 has a V:Bi:W ratio of 10:1:1, Catalyst 2 has a V:Bi:W ratio of 1:1:1, and Catalyst 3 has a V:Bi:W ratio of 6:3:1, and Catalyst 4 has a V:Bi:W ratio of 10:5:1. As shown in Table 11, catalysts with V:Bi:W ratio of 10:1:1 and 1:1:1 have similar surface area, pore volume and pore size. As shown, when the V:Bi:W ratio is 6:3:1 or 10:5:1, the surface area, pore volume, and pore size, surprisingly and unexpectedly increased. This suggests that the relative levels of V:Bi:W:P as well as the preparation conditions may have a significant impact on catalyst surface area, pore volume and pore size.

TABLE 11
Catalyst Compositions

| | | | BET | BET | BET |
|---|---|---|---|---|---|
| | | | Surface | Ave. | Ave. |
| | Catalyst | | Area | Pore Vol. | Pore Size |
| Catalyst | Formula | Preparation Details | (m²/g) | (cm³) | (nm) |
| 1 | V₁₀BiWP_{13.8}O₅₈ | citric acid, ethylene glycol, | 7.3 | 0.03 | 13.7 |
| | | 5.8% SiO₂, 2% methylcellulose | | | |
| 2 | VBiWP_{3.45}O₁₃ | citric acid, ethylene glycol, | 7.3 | 0.02 | 11.8 |
| | | 5.8% SiO₂, 2% methylcellulose | | | |
| 3 | V₆Bi₃WP_{11.5}O₄₆ | citric acid, ethylene glycol, | 13.3 | 0.04 | 12.4 |
| | | 5.8% SiO₂, 3% methylcellulose | | | |
| 4 | V₁₀Bi₅WP_{18.4}O₇₄ | citric acid, ethylene glycol, | 17.3 | 0.06 | 13.8 |
| | | 5.8% SiO₂, 2% methylcellulose | | | |

### Example 11

A reaction feed comprising acetic acid, formaldehyde, methanol, water, oxygen, and nitrogen was passed through a fixed bed reactor comprising Catalysts 1-4 shown in Table 11. The reactions for Catalysts 1-4 were conducted at a reactor temperature of 370° C. and a GHSV of 600 Hr⁻¹, total organics of 8 mole %, acetic acid and formaldehyde ratio of 1.5, O₂ of 1.0%, H₂O of 4.4 mole %, total N₂ of 87 mole %, and formalin equivalent of 55%. Acrylic acid and methyl acrylate (collectively, “acrylate product”) were produced. Acetic acid conversion, acrylate selectivity, acrylate yield, and acrylate space time yield were measured for Catalysts 1-4 at various time points of the reaction. A commercial VPO catalyst Comp. A was also tested under the same condition. The results are shown in Table 12.

TABLE 12
Acrylate Production

| | | HOAc | Acrylate | | |
|---|---|---|---|---|---|
| | Runtime | Conv. | Selectivity | Acrylate | Acrylate STY |
| Catalyst | (h) | (%) | (%) | Yield (%) | (g/hr/L) |
| 1 | 0.6 | 55 | 77 | 42 | 40 |
| | 1.8 | 51 | 76 | 39 | 37 |
| | 3.7 | 48 | 74 | 36 | 34 |
| | 4.5 | 47 | 74 | 35 | 32 |
| | 22.8 | 39 | 72 | 28 | 26 |
| | 23.5 | 39 | 72 | 28 | 26 |
| | 24.1 | 40 | 71 | 28 | 26 |
| | 24.9 | 40 | 71 | 28 | 26 |
| 2 | 0.7 | 53 | 77 | 41 | 39 |
| | 1.3 | 51 | 82 | 42 | 41 |
| | 2.0 | 54 | 85 | 46 | 44 |
| | 18.5 | 48 | 81 | 39 | 38 |
| | 19.2 | 47 | 81 | 38 | 36 |
| | 20.3 | 47 | 82 | 39 | 37 |
| 3 | 0.7 | 65 | 79 | 52 | 50 |
| | 1.9 | 61 | 79 | 48 | 46 |
| | 4.0 | 57 | 80 | 45 | 44 |
| | 4.7 | 54 | 79 | 42 | 41 |
| | 24.1 | 43 | 74 | 32 | 30 |
| | 26.2 | 40 | 82 | 32 | 31 |
| | 27.4 | 40 | 79 | 32 | 30 |
| | 28.1 | 41 | 74 | 30 | 29 |
| 4 | 0.6 | 64 | 78 | 50 | 48 |
| | 1.8 | 60 | 79 | 48 | 46 |
| | 3.7 | 56 | 78 | 44 | 43 |
| | 4.5 | 53 | 78 | 41 | 40 |
| | 22.8 | 43 | 72 | 31 | 30 |
| | 23.5 | 42 | 72 | 31 | 30 |
| | 24.1 | 42 | 73 | 31 | 30 |
| | 24.9 | 42 | 73 | 30 | 30 |
| VPO | 0.8 | 39 | 85 | 33 | 31 |
| Comp. A | 2.3 | 32 | 85 | 27 | 25 |
| | 4.0 | 32 | 85 | 27 | 26 |
| | 5.8 | 28 | 82 | 23 | 22 |
| | 23.3 | 25 | 79 | 20 | 19 |
| | 24.9 | 23 | 84 | 20 | 19 |

Acetic acid conversion, acrylate selectivity, acrylate yield, and acrylate space time yield were measured for Catalysts 1-4 and Comp. A at various time points of the reaction. Catalysts 1-4, all of which contain bismuth and tungsten, unexpectedly outperform Comp. A, which is conventional bismuth-free and tungsten-free commercially available vanadium catalysts. Catalysts 1-4 show better acetic acid conversions, acrylate yield, and acrylate STY than Comp. A. Specifically, Catalysts 3 and 4 show high initial acetic acid conversion of 65% and 64%, respectively, and high acrylic acid selectivity of 79% and 78%, respectively. In comparison, Comp. A has an initial acetic acid conversion of 39% and decreased to 23% over the course of 24.9 hours. The acrylate yield of Comp. A also decreased from 33% to 20% over the course of 24.9 hours. Furthermore, the acrylate STY of Comp. A decreased from 31 g/hr/L to 19 g/hr/L. Therefore, as shown by the data, Catalysts 1-4 outperform commercially available VPO catalyst.

### Example 12

Table 13 shows surface area, pore volume, and pore size of catalysts 5-7, which comprise vanadium, bismuth, and tungsten. Examples 5-7 were prepared via the preparation method of Example 8, but different calcinations temperatures, e.g., in the last step of the temperature profile, were employed.

TABLE 13
Catalyst Compositions

| | | | | | BET |
|---|---|---|---|---|---|
| | | | BET | BET | Ave. |
| | | | Surface | Ave. Pore | Pore |
| | Catalyst | Calcination | Area | Vol. | Size |
| Catalyst | Formula | Temperature | (m²/g) | (cm³) | (nm) |
| 5 | V₁₀Bi₅WP_{18.4}O₇₄ | 450 | 16.0 | 0.05 | 12.6 |
| 6 | V₁₀Bi₅WP_{18.4}O₇₄ | 500 | 9.1 | 0.03 | 13.6 |
| 7 | V₁₀Bi₅WP_{18.4}O₇₄ | 550 | 5.3 | 0.02 | 13.6 |

The effect of calcination temperature on V₁₀Bi₅WP_{18.4}O₇₄ was studied. As shown in Table 13, Catalysts 5-7 have the same formula but each was calcinated at a different temperature. Catalysts Comp. A, Comp. B, and Comp. C are commercially available vanadium catalysts that do not contain bismuth or tungsten. As shown in Table 13, as the calcination temperature increases, the surface area and pore volume of the catalyst decreases, whereas the pore size slightly increases. This suggests that calcination temperature may have a significant impact on catalyst surface area and pore volume.

### Example 13

A reaction feed comprising acetic acid, formaldehyde, methanol, water, oxygen, and nitrogen was passed through a fixed bed reactor comprising Catalysts 5-7 shown in Table 14. The reactions for Catalysts 5-7 were conducted at a reactor temperature of 380° C. and a GHSV of 2400 Hr⁻¹, total organics of 18 mole %, acetic acid and formaldehyde ratio of 1.5, O₂ of 3.3%, H₂O of 6.4 mole %, total N₂ of 72 mole %, and formalin equivalent of 65%. Acrylic acid and methyl acrylate (collectively, “acrylate product”) were produced. Acetic acid conversion, acrylate selectivity, acrylate yield, and acrylate space time yield were measured for Catalysts 5-7 at various time points of the reaction. A commercial VPO catalyst Comp. A was also tested under the same condition. The results are shown in Table 14.

TABLE 14
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 5 | 0.5 | 52 | 78 | 41 | 350 |
| | 1.3 | 50 | 80 | 40 | 343 |
| | 2.2 | 49 | 79 | 39 | 336 |
| | 3.2 | 49 | 80 | 39 | 333 |
| | 23.3 | 48 | 80 | 39 | 331 |
| | 24.4 | 48 | 80 | 38 | 326 |
| | 25.7 | 47 | 80 | 38 | 322 |
| | 26.6 | 46 | 80 | 37 | 317 |
| | 47.0 | 42 | 81 | 34 | 291 |
| | 49.6 | 42 | 81 | 34 | 289 |
| | 50.9 | 41 | 82 | 34 | 287 |
| | 52.2 | 41 | 82 | 33 | 287 |
| | 71.3 | 40 | 82 | 33 | 283 |
| | 72.1 | 40 | 79 | 31 | 269 |
| | 75.8 | 40 | 80 | 32 | 277 |
| | 76.1 | 40 | 84 | 33 | 285 |
| 6 | 0.5 | 43 | 81 | 35 | 296 |
| | 1.3 | 45 | 82 | 36 | 312 |
| | 1.9 | 43 | 82 | 35 | 301 |
| | 19.1 | 42 | 82 | 34 | 295 |
| | 20.8 | 44 | 83 | 36 | 312 |
| | 21.9 | 43 | 83 | 36 | 307 |
| | 23.3 | 44 | 83 | 36 | 310 |
| | 24.0 | 44 | 83 | 36 | 309 |
| | 43.0 | 44 | 83 | 36 | 309 |
| | 43.7 | 43 | 83 | 36 | 305 |
| | 44.4 | 43 | 83 | 36 | 307 |
| 7 | 0.5 | 36 | 83 | 30 | 258 |
| | 1.3 | 36 | 85 | 31 | 265 |
| | 2.2 | 37 | 85 | 31 | 268 |
| | 3.2 | 36 | 86 | 31 | 268 |
| | 23.3 | 36 | 86 | 30 | 262 |
| | 24.4 | 36 | 86 | 31 | 265 |
| | 25.7 | 36 | 84 | 31 | 263 |
| | 26.8 | 36 | 86 | 31 | 265 |
| | 47.0 | 34 | 87 | 29 | 250 |
| | 49.6 | 34 | 87 | 29 | 251 |
| | 50.9 | 34 | 85 | 29 | 252 |
| | 52.2 | 34 | 86 | 29 | 249 |
| | 71.3 | 34 | 87 | 29 | 251 |
| | 72.1 | 34 | 85 | 29 | 253 |
| | 75.8 | 34 | 88 | 30 | 256 |
| | 76.1 | 33 | 88 | 29 | 253 |
| VPO | 0.8 | 39 | 85 | 33 | 31 |
| Comp. A | 2.3 | 32 | 85 | 27 | 25 |
| | 4.0 | 32 | 85 | 27 | 26 |
| | 5.8 | 28 | 82 | 23 | 22 |
| | 23.3 | 25 | 79 | 20 | 19 |
| | 24.9 | 23 | 84 | 20 | 19 |

Catalysts 5-7, all of which contain bismuth and tungsten, unexpectedly outperform Comp. A, which is conventional bismuth-free and tungsten-free commercially available vanadium catalysts. For example, Catalysts 5-7 demonstrate average acetic acid conversions of 45%, 43%, and 35%, respectively, while Comp. A demonstrates an average acetic acid conversion of only 28%. Also, Catalysts 5-7 demonstrate average acrylate STY of 308 g/hr/L, 306 g/hr/L, and 258 g/hr/L, respectively, while Comp. A demonstrates an average yield of only 205 g/hr/L. In addition, Catalysts 5-7 demonstrate average acrylate yields of 36%, 36%, and 30%, respectively, while Comp. A demonstrates an average yield of only 22%.

Surprisingly and unexpectedly, as shown, Catalysts 6 and 7 maintained steady acetic acid conversion, acrylate selectivity, acrylate yield, and acrylate space time yield over long time periods, i.e., Catalysts 6 and 7 showed little if any catalyst deactivation. For example, over a 44.4 hour period, the acetic acid conversion for Catalyst 6 remains between 42% and 45%. Similarly, over a 76.1 hour period, the acetic acid conversion for Catalyst 7 remains between 33% and 37%. Acetic acid conversion, acrylate yield, and acrylate space time yield for Comp. A decreased after only 2.3 hours. In comparison, Catalysts 6 and 7 have acetic acid conversion from 42% to 45% and 35%-37%, acrylate yield from 81% to 83% and from 83% to 88%, and acrylate space time yield from 295 g/hr/L to 312 g/hr/L and 249 g/hr/L to 268 g/hr/L, respectively for an extended amount of time.

As demonstrated, over longer time periods, e.g., for greater than 40 hours, Catalysts 5-7 continued to demonstrate maintenance of steady reaction performance, as compared to Catalyst Comp. A, which deactivated significantly over 24.9 hours. For example, for Comp. A, the acetic acid conversion dropped from 33% to 26%, acrylate yield dropped from 25% to 20%, and the space time yield dropped from 185 g/hr/L to 233 g/hr/L.

As shown, as the catalyst calcination temperature increases, the acetic acid conversion for the catalyst decreases. For example, Catalyst 5 has an initial acetic acid conversion of 52%, and Catalysts 6 and 7 have an initial acetic acid conversion of 43% and 35%, respectively. As discussed above, Catalyst 5 has a larger surface area than Catalysts 6 and 7. Without being bound by theory, it appears that surface area may impact the acetic acid conversion of the catalysts.

### Example 14

Table 15 shows surface area, pore volume, and pore size of catalysts 8 and 9 comprising vanadium, bismuth, and tungsten. Catalysts 8 and 9 were prepared via the preparation method of Example 1 but different methylcellulose concentrations were utilized. Catalysts Comp. B-D are shown as comparative examples. The effect of methylcellulose content was studied. Catalysts 8 and 9 were prepared using 3% and 10% methyl cellulose, respectively. As shown in Table 15, the increase of methyl cellulose has little, if any, effect on the surface area, the pore volume and pore size of the catalyst.

TABLE 15
Catalyst Compositions

| | | | BET | BET Ave. | BET Ave. |
|---|---|---|---|---|---|
| | | Methyl | Surface | Pore Vol. | Pore Size |
| Catalyst | Catalyst Formula | Cellulose | Area (m²/g) | (cm³) | (nm) |
| 8 | V₁₀Bi₅WP_{18.4}O₇₄ | 3% | 15 | 0.038 | 10 |
| 9 | V₁₀Bi₅WP_{18.4}O₇₄ | 10% | 15.5 | 0.053 | 13.7 |
| Comp. B | VPO | Commercial | n/a | n/a | n/a |
| | | VPO | | | |
| Comp. C | VPO | citric acid, | 8 | 0.03 | 12 |
| | | ethylene glycol, | | | |
| | | 5.0% SiO₂, 10% | | | |
| | | methylcellulose | | | |
| Comp. D | VPO | (V₂O₅ + | 14.0 | 0.05 | 13 |
| | | isobutanol) + | | | |
| | | H₃PO₄ | | | |

### Example 15

A reaction feed comprising acetic acid, formaldehyde, methanol, water, oxygen, and nitrogen was passed through a fixed bed reactor comprising Catalysts 8 and 9 shown in Table 15. The reactions for Catalysts 8 and 9 were conducted at a reactor temperature of 380° C. and a GHSV of 2000 Hr⁻¹, total organics of 32 mole %, acetic acid and formaldehyde ratio of 1.5, O₂ of 5.7%, H₂O of 7.2 mole %, total N₂ of 55 mole %, and formalin equivalent of 75%. Catalysts Comp. B-Comp. D are also included as comparisons. The reactions for Catalysts Comp. B-Comp. D were conducted at a reactor temperature of 375° C. and a GHSV of 2000 Hr⁻¹, total organics of 32 mole %, acetic acid and formaldehyde ratio of 1.5, O₂ of 4.8%, H₂O of 7.2 mole %, total N₂ of 56 mole %, and formalin equivalent of 75%. Acrylic acid and methyl acrylate (collectively, “acrylate product”) were produced. Acetic acid conversion, acrylate selectivity, acrylate yield, and acrylate space time yield were measured for Catalysts 8, 9, Comp. B, Comp. C and Comp. D at various time points of the reaction. The results are shown in Table 16.

TABLE 16
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 8 | 0.6 | 34 | 88 | 30 | 396 |
| | 1.8 | 34 | 88 | 30 | 397 |
| | 2.7 | 34 | 88 | 30 | 396 |
| | 3.7 | 34 | 88 | 30 | 396 |
| | 4.6 | 34 | 87 | 30 | 393 |
| 9 | 0.5 | 40 | 87 | 35 | 400 |
| | 1.8 | 39 | 88 | 35 | 400 |
| | 3.0 | 39 | 88 | 34 | 398 |
| | 22.6 | 40 | 83 | 33 | 385 |
| | 23.8 | 41 | 80 | 33 | 381 |
| | 25.0 | 39 | 87 | 34 | 396 |
| | 26.2 | 41 | 81 | 33 | 384 |
| | 27.2 | 39 | 87 | 34 | 396 |
| | 51.0 | 39 | 86 | 34 | 394 |
| | 69.0 | 39 | 87 | 34 | 389 |
| | 70.9 | 41 | 79 | 32 | 376 |
| | 72.1 | 41 | 79 | 33 | 377 |
| | 76.5 | 39 | 86 | 34 | 391 |
| Comp. B | 0.8 | 27 | 85 | 23 | 304 |
| | 1.7 | 24 | 94 | 22 | 289 |
| | 2.7 | 23 | 95 | 22 | 280 |
| | 3.9 | 22 | 97 | 21 | 277 |
| Comp. C | 0.8 | 22 | 90 | 20 | 255 |
| | 1.7 | 22 | 90 | 19 | 253 |
| | 2.7 | 22 | 87 | 19 | 251 |
| | 3.9 | 22 | 90 | 19 | 254 |
| Comp. D | 1.2 | 39 | 77 | 30 | 391 |
| | 2.3 | 38 | 76 | 29 | 378 |
| | 3.3 | 37 | 77 | 28 | 369 |
| | 18.0 | 34 | 73 | 24 | 319 |
| | 19.3 | 33 | 72 | 24 | 310 |
| | 20.4 | 31 | 79 | 24 | 315 |
| | 21.4 | 31 | 78 | 24 | 310 |
| | 22.5 | 30 | 78 | 24 | 309 |

Catalysts 8 and 9, each of which contain bismuth and tungsten, unexpectedly outperform Comp. B, Comp. C and Comp. D, which are conventional bismuth-free and tungsten-free commercially available vanadium catalysts. For example, Catalysts 8 and 9 demonstrate average acetic acid conversions of 34% and 40%, respectively, while Comp. B, Comp. C and Comp. D demonstrate an average acetic acid conversion of only 24%, 22%, and 34%, respectively. Also, Catalysts 8 and 9 demonstrate average acrylate STY of 396 g/hr/L and 390 g/hr/L, respectively, while Comp. B, Comp. C and Comp. D demonstrate average yields of only 287 g/hr/L, 254 g/hr/L and 338 g/hr/L, respectively. In addition, Catalysts 8 and 9 demonstrate average acrylate yields of 30% and 34%, respectively, while Comp. B, Comp. C, and Comp. D demonstrate an average yield of only 22%, 19%, and 26%, respectively.

In addition, the Catalysts 8 and 9 show steady acetic acid conversion, acrylate selectivity, acrylate yield, and acrylate STY over a long period of time. For example, Catalyst 8 has a steady 34% acetic acid conversion, 87-88% acrylate selectivity, a steady 30% acrylate yield and 393-397 g/hr/L acrylate STY over a 4.6 hour period. Catalyst 9 has a 39-41% acetic acid conversion, 79-88% acrylate selectivity, 32-35% yield and 376-400 g/hr/L acrylate STY over a 76.5 hour period. This shows that both catalysts have no to little deactivation over a long period of time. In addition, it appears that a higher amount of methylcellulose, e.g., 10%, increases the acetic acid conversion of the reaction. For example, the average conversion for Catalyst 9 is 40% in comparison to Catalyst 8, which has an average conversion of 34%. In comparison, acetic acid conversion, acrylate yield, and acrylate space time yield for Comp. B and Comp. D decreased after only 3.9 hours and 3.3 hours, respectively. Although Catalyst Comp. C shows a steady acetic acid conversion, acrylate selectivity, acrylate yield, and acrylate space time yield for a 4 hour period, the acetic acid conversion is at an undesirable 22%. In comparison, Catalysts 8 and 9 have acetic acid conversion of a steady 34% and 39%-41%, respectively, for an extended amount of time.

### Example 16

Table 17 shows surface area, pore volume, and pore size of catalysts 10-12 comprising vanadium, bismuth, and tungsten. Catalysts 10-12 were prepared via the preparation method of Example 8 but different phosphorus levels were utilized.

TABLE 17
Catalyst Compositions

| | | | | BET Ave. | BET Ave. |
|---|---|---|---|---|---|
| | Catalyst | | BET Surface | Pore Vol. | Pore Size |
| Catalyst | Formula | Preparation Details | Area (m²/g) | (cm³) | (nm) |
| 10 | V₁₀Bi₅WP₁₅O₆₃ | citric acid, ethylene | 12.8 | 0.041 | 12.8 |
| | | glycol, 7% SiO₂, 10% | | | |
| | | methylcellulose | | | |
| 11 | V₁₀Bi₅WP_{16.5}O₆₈ | citric acid, ethylene | 21.1 | 0.071 | 13.5 |
| | | glycol, 7% SiO₂, 10% | | | |
| | | methylcellulose | | | |
| 12 | V₁₀Bi₅WP_{18.4}O₇₄ | citric acid, ethylene | 20.4 | 0.079 | 15.5 |
| | | glycol, 10% SiO₂, 10% | | | |
| | | methylcellulose | | | |

The effect of phosphorus level was studied. Catalysts 10, 11 and 12 have different levels of phosphorus as indicated in the chemical formulas with P=15, 16.5 and 18.4, respectively. As shown in Table 17, the increase of phosphorus level appears to increase the surface area, pore volume and pore size of the catalysts. Surprisingly and unexpectedly, when the phosphorus atomic number is maintained in the range of 15 to 20.4, significantly higher surface areas are achieved.

### Example 17

A reaction feed comprising acetic acid, formaldehyde, methanol, water, oxygen, and nitrogen was passed through a fixed bed reactor comprising Catalysts 10, 11 and 12 shown in Table 17. The reactions for Catalysts 8 and 9 were conducted at a reactor temperature of 375° C. and a GHSV of 2000 Hr⁻¹, total organics of 32 mole %, acetic acid and formaldehyde ratio of 1.5, O₂ of 4.8%, H₂O of 7.0 mole %, total N₂ of 56 mole %, and formalin equivalent of 75%. Catalysts Comp. B-Comp. D are also included as comparisons. The reaction conditions of Comp. B-Comp. D were the same as Example 16. Acrylic acid and methyl acrylate (collectively, “acrylate product”) were produced. Acetic acid conversion, acrylate selectivity, acrylate yield, and acrylate space time yield were measured for Catalysts 10, 11, 12, Comp. B, Comp. C and Comp. D at various time points of the reaction. The results are shown in Table 18.

TABLE 18
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 10 | 1.6 | 43 | 76 | 33 | 412 |
| | 2.7 | 42 | 77 | 32 | 408 |
| | 3.8 | 41 | 78 | 32 | 409 |
| | 5.3 | 41 | 80 | 33 | 413 |
| | 21.4 | 41 | 77 | 31 | 398 |
| | 24.0 | 41 | 77 | 32 | 399 |
| | 25.2 | 41 | 76 | 31 | 397 |
| 11 | 1.6 | 43 | 76 | 33 | 427 |
| | 2.7 | 42 | 76 | 32 | 423 |
| | 3.8 | 43 | 75 | 32 | 419 |
| | 5.3 | 42 | 75 | 32 | 418 |
| | 21.4 | 40 | 76 | 31 | 400 |
| | 22.7 | 41 | 76 | 31 | 411 |
| | 24.0 | 41 | 77 | 31 | 410 |
| | 25.2 | 41 | 77 | 32 | 415 |
| 12 | 0.8 | 41 | 88 | 36 | 480 |
| | 1.8 | 38 | 88 | 33 | 439 |
| | 2.6 | 35 | 90 | 31 | 409 |
| | 4.5 | 34 | 90 | 30 | 399 |
| | 5.5 | 41 | 86 | 35 | 463 |
| | 22.7 | 36 | 89 | 32 | 423 |
| | 23.7 | 35 | 90 | 31 | 416 |
| | 24.7 | 36 | 89 | 32 | 427 |
| Comp. B | 0.8 | 27 | 85 | 23 | 304 |
| | 1.7 | 24 | 94 | 22 | 289 |
| | 2.7 | 23 | 95 | 22 | 280 |
| | 3.9 | 22 | 97 | 21 | 277 |
| Comp. C | 0.8 | 22 | 90 | 20 | 255 |
| | 1.7 | 22 | 90 | 19 | 253 |
| | 2.7 | 22 | 87 | 19 | 251 |
| | 3.9 | 22 | 90 | 19 | 254 |
| Comp. D | 1.2 | 39 | 77 | 30 | 391 |
| | 2.3 | 38 | 76 | 29 | 378 |
| | 3.3 | 37 | 77 | 28 | 369 |
| | 18.0 | 34 | 73 | 24 | 319 |
| | 19.3 | 33 | 72 | 24 | 310 |
| | 20.4 | 31 | 79 | 24 | 315 |
| | 21.4 | 31 | 78 | 24 | 310 |
| | 22.5 | 30 | 78 | 24 | 309 |

Catalysts 10-12, all of which contain bismuth and tungsten, unexpectedly outperform Comp. B, Comp. C and Comp. D, which are conventional bismuth-free and tungsten-free commercially available vanadium catalysts. For example, Catalysts 10-12 demonstrate average acetic acid conversions of 41%, 42%, and 37%, respectively, while Comp. B, Comp. C and Comp. D demonstrate an average acetic acid conversion of only 24%, 22%, and 34%, respectively. Also, Catalysts 10-12 demonstrate average acrylate STY of 405 g/hr/L, 415 g/hr/L, and 432 g/hr/L, respectively, while Comp. B, Comp. C and Comp. D demonstrate average yields of only 287 g/hr/L, 254 g/hr/L and 338 g/hr/L, respectively. In addition, Catalysts 10-12 demonstrate average acrylate yields of 32%, 32%, and 33%, respectively, while Comp. B, Comp. C, and Comp. D demonstrate an average yield of only 22%, 19%, and 26%, respectively.

As shown in Table 18, Catalysts 10, 11 and 12 show steady acetic acid conversion, acrylate selectivity, acrylate yield, and acrylate STY over a 24 hour period. For example, Catalyst 10 has a 41-43% acetic acid conversion, 76-80% acrylate selectivity, 31-32% acrylate yield and 397-413 g/hr/L acrylate STY over a 25.2 hour period. Catalyst 11 has a 40-43% acetic acid conversion, 75-77% acrylate selectivity, 31-32% acrylate yield and 400-427 g/hr/L acrylate STY over a 25.2 hour period. Catalyst 12 has a 34-41% acetic acid conversion, 86-90% acrylate selectivity, 30-36% acrylate yield and 399-480 g/hr/L acrylate STY over a 24.7 hour period. In comparison, acetic acid conversion, acrylate yield, and acrylate space time yield for Comp. B and Comp. D decreased after only 3.9 hours and 3.3 hours, respectively. Although Catalyst Comp. D shows a steady acetic acid conversion, acrylate selectivity, acrylate yield, and acrylate space time yield for a 4 hour period, the acetic acid conversion is at an undesirable 22%. This shows that all three catalysts of the present invention have little deactivation over a long period of time.

In addition, it appears that the change of phosphorus level has minor effect on acetic acid conversion. For example, Catalysts 10 and 11 with phosphorus level of 15 and 16.5, respectively, appear to have slightly better acetic acid conversion than Catalyst 12 having a phosphorus level of 18.4.

### Example 18

Table 19 shows surface area, pore volume, and pore size of catalysts 13 and 14 comprising vanadium, bismuth, and tungsten. Catalyst 13 was prepared via the preparation method of Example 9 with reduced or unreduced VOPO₄. Catalyst 14 was prepared by refluxing V₂O₅ in diluted H₃PO₄ for 24 hours and was allowed to cool to room temperature. The catalyst was collected via centrifugation or filtration. The unreduced vanadium phosphate was combined with ammonium metatungstate, and BiPO₄ that was prepared from the addition of 43% H₃PO₄ to a bismuth nitrate (10% HNO₃) solution. The mixture was ground vigorously by hand or ball milled for 5 hours and the mixture was calcined following the calcinations scheme provided in Example 9.

TABLE 19
Catalyst Compositions

| | | | | | BET |
|---|---|---|---|---|---|
| | | | BET | BET Ave. | Ave. |
| | | | Surface | Pore | Pore |
| | | Preparation | Area | Vol. | Size |
| Catalyst | Catalyst Formula | Method | (m²/g) | (cm³) | (nm) |
| 13 | V₁₀Bi₅WP_{16.9}O₆₉ | Grind with | 8.9 | 0.027 | 12.1 |
| | | reduced | | | |
| | | VOPO₄ | | | |
| 14 | V₁₀Bi₅WP_{16.9}O₆₉ | Grind with | 5.1 | 0.010 | 7.4 |
| | | unreduced | | | |
| | | VOPO₄ | | | |

The effect of reduced VOPO₄ versus unreduced VOPO₄ was studied. As shown in Table 19, Catalysts 13 and 14 were prepared by physically mixing powdered form of the metal precursors. As shown in Table 19, Catalyst 13 with reduced VOPO₄ has a larger surface area, pore volume, and pore size than Catalyst 14 with unreduced VOPO₄.

### Example 19

A reaction feed comprising acetic acid, formaldehyde, methanol, water, oxygen, and nitrogen was passed through a fixed bed reactor comprising Catalysts 13 and 14 shown in Table 19. The reactions for Catalysts 13 and 14 were conducted at a reactor temperature of 375° C. and a GHSV of 2000 Hr⁻¹, total organics of 32 mole %, acetic acid and formaldehyde ratio of 1.5, O₂ of 4.8%, H₂O of 7.1 mole %, total N₂ of 56 mole %, and formalin equivalent of 75%. Catalysts Comp. B-Comp. D are also included as comparisons. The reaction conditions of Comp. B-Comp. D were the same as Example 17. Acrylic acid and methyl acrylate (collectively, “acrylate product”) were produced. Acetic acid conversion, acrylate selectivity, acrylate yield, and acrylate space time yield were measured for Catalysts 13, 14, Comp. B, Comp. C and Comp. D at various time points of the reaction. The results are shown in Table 20.

TABLE 20
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 13 | 2.1 | 35 | 78 | 27 | 353 |
| | 3.3 | 36 | 79 | 28 | 366 |
| | 4.5 | 37 | 78 | 29 | 370 |
| | 5.4 | 37 | 78 | 29 | 371 |
| 14 | 1.6 | 28 | 85 | 24 | 298 |
| | 2.4 | 30 | 84 | 25 | 305 |
| | 3.3 | 30 | 83 | 25 | 311 |
| | 4.2 | 31 | 83 | 25 | 312 |
| | 20.8 | 32 | 83 | 26 | 324 |
| | 21.6 | 32 | 83 | 27 | 330 |
| | 22.5 | 33 | 83 | 27 | 333 |
| | 23.4 | 32 | 83 | 27 | 333 |
| Comp. B | 0.8 | 27 | 85 | 23 | 304 |
| | 1.7 | 24 | 94 | 22 | 289 |
| | 2.7 | 23 | 95 | 22 | 280 |
| | 3.9 | 22 | 97 | 21 | 277 |
| Comp. C | 0.8 | 22 | 90 | 20 | 255 |
| | 1.7 | 22 | 90 | 19 | 253 |
| | 2.7 | 22 | 87 | 19 | 251 |
| | 3.9 | 22 | 90 | 19 | 254 |
| Comp. D | 1.2 | 39 | 77 | 30 | 391 |
| | 2.3 | 38 | 76 | 29 | 378 |
| | 3.3 | 37 | 77 | 28 | 369 |
| | 18.0 | 34 | 73 | 24 | 319 |
| | 19.3 | 33 | 72 | 24 | 310 |
| | 20.4 | 31 | 79 | 24 | 315 |
| | 21.4 | 31 | 78 | 24 | 310 |
| | 22.5 | 30 | 78 | 24 | 309 |

Catalysts 13 and 14, all of which contain bismuth and tungsten, unexpectedly outperform Comp. B, Comp. C and Comp. D, which are conventional bismuth-free and tungsten-free commercially available vanadium catalysts. For example, Catalysts 13 and 14 demonstrate average acetic acid conversions of 36% and 31%, respectively, while Comp. B, Comp. C and Comp. demonstrate an average acetic acid conversion of only 24%, 22%, and 34%, respectively. Also, Catalysts 13 and 14 demonstrate average acrylate STY of 365 g/hr/L and 318 g/hr/L, respectively, while Comp. B, Comp. C and Comp. D demonstrate average yields of only 287 g/hr/L, 254 g/hr/L and 338 g/hr/L, respectively. In addition, Catalysts 13 and 14 demonstrate average acrylate yields of 28% and 26%, respectively, while Comp. B, Comp. C, and Comp. D demonstrate an average yield of only 22%, 19%, and 26%, respectively.

As shown in Table 20, Catalysts 13 and 14 show steady acetic acid conversion, acrylate selectivity, acrylate yield, and acrylate STY. For example, Catalyst 13 has a 35-38% acetic acid conversion, 78-79% acrylate selectivity, 27-29% acrylate yield and 353-371 g/hr/L acrylate STY over a 5.4 hour period. Catalyst 14 has a 28-33% acetic acid conversion, 83-85% acrylate selectivity, 24-27% yield and 298-333 g/hr/L acrylate STY over a 23.4 hour period. This shows that both catalysts have little deactivation over a long period of time. In comparison, acetic acid conversion, acrylate yield, and acrylate space time yield for Comp. B and Comp. D decreased after only 3.9 hours and 3.3 hours, respectively. Although Catalyst Comp. C shows a steady acetic acid conversion, acrylate selectivity, acrylate yield, and acrylate space time yield for a 4 hour period, the acetic acid conversion is at an undesirable 22%.

In addition, it appears that Catalyst 13 with reduced VOPO₄ has a higher acetic acid conversion than Catalyst 14 with unreduced VOPO₄.

### Example 20

Table 21 shows surface area, pore volume, and pore size of catalysts 15 and 16 comprising vanadium and a doping amount of bismuth and tungsten. Catalysts 15 and 16 were prepared via the preparation method of Example 8.

TABLE 21
Catalyst Compositions

| | | | | BET Ave. | BET Ave. |
|---|---|---|---|---|---|
| | | Preparation | BET Surface | Pore Vol. | Pore Size |
| Catalyst | Catalyst Formula | Method | Area (m²/g) | (cm³) | (nm) |
| 15 | V₁₀Bi_{0.16}W_{0.5}P_{12.16}O₅₃ | citric acid, 10% | 7 | 0.019 | 12 |
| | | methylcellulose, | | | |
| | | 2.5% SiO₂ | | | |
| 16 | V₁₀Bi_{0.16}W_{0.5}P_{11.7}O₅₁ | citric acid, 10% | 26 | 0.079 | 12 |
| | | methylcellulose, | | | |
| | | 2.5% SiO₂ | | | |

The effect of Bi and W doping was studied. As shown in Table 21, Catalysts 15 and 16 contain a reduced level of Bi and W. For example, Catalysts 15 and 16 have a V:Bi:W ratio of 10:0.16:0.5. Catalysts 15 and 16 also have different levels of phosphorus and oxygen. As shown in Table 12, Catalyst 16 has a much larger surface area and pore volume than Catalyst 16.

### Example 21

A reaction feed comprising acetic acid, formaldehyde, methanol, water, oxygen, and nitrogen was passed through a fixed bed reactor comprising Catalysts 15 and 16 shown in Table 21 and Comp. B-Comp. D shown in Table 4 above. The reactions for all catalysts were conducted at a reactor temperature of 375° C. and a GHSV of 2000 Hr⁻¹, total organics of 32 mole %, acetic acid and formaldehyde ratio of 1.5, O₂ of 4.8%, H₂O of 7.2 mole %, total N₂ of 56 mole %, and formalin equivalent of 75%. Catalysts Comp. B-Comp. D are also included as comparisons. The reaction conditions of Comp. B-Comp. D were the same as Example 15. Acrylic acid and methyl acrylate (collectively, “acrylate product”) were produced. Acetic acid conversion, acrylate selectivity, acrylate yield, and acrylate space time yield were measured for Catalysts 15, 16, Comp. B, Comp. C and Comp. D at various time points of the reaction. The results are shown in Table 22.

TABLE 22
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 15 | 0.9 | 32 | 79 | 26 | 333 |
| | 1.4 | 32 | 78 | 25 | 328 |
| | 17.4 | 27 | 84 | 23 | 301 |
| | 18.7 | 29 | 78 | 23 | 298 |
| | 19.7 | 29 | 78 | 23 | 298 |
| | 20.8 | 29 | 77 | 23 | 294 |
| | 21.8 | 29 | 77 | 23 | 294 |
| 16 | 0.9 | 31 | 88 | 27 | 351 |
| | 1.4 | 30 | 89 | 27 | 350 |
| | 17.4 | 29 | 87 | 25 | 328 |
| | 18.7 | 29 | 88 | 25 | 328 |
| | 19.7 | 29 | 87 | 25 | 329 |
| | 20.8 | 29 | 88 | 25 | 329 |
| | 21.8 | 29 | 88 | 25 | 330 |
| Comp. B | 0.8 | 27 | 85 | 23 | 304 |
| | 1.7 | 24 | 94 | 22 | 289 |
| | 2.7 | 23 | 95 | 22 | 280 |
| | 3.9 | 22 | 97 | 21 | 277 |
| Comp. C | 0.8 | 22 | 90 | 20 | 255 |
| | 1.7 | 22 | 90 | 19 | 253 |
| | 2.7 | 22 | 87 | 19 | 251 |
| | 3.9 | 22 | 90 | 19 | 254 |
| Comp. D | 1.2 | 39 | 77 | 30 | 391 |
| | 2.3 | 38 | 76 | 29 | 378 |
| | 3.3 | 37 | 77 | 28 | 369 |
| | 18.0 | 34 | 73 | 24 | 319 |
| | 19.3 | 33 | 72 | 24 | 310 |
| | 20.4 | 31 | 79 | 24 | 315 |
| | 21.4 | 31 | 78 | 24 | 310 |
| | 22.5 | 30 | 78 | 24 | 309 |

Catalysts 15 and 16, all of which contain bismuth and tungsten, unexpectedly outperform Comp. B, Comp. C and Comp. D, which are conventional bismuth-free and tungsten-free commercially available vanadium catalysts. For example, Catalysts 15 and 16 demonstrate average acetic acid conversions of 30% and 29%, respectively, while Comp. B, Comp. C and Comp. D demonstrate an average acetic acid conversion of only 24%, 22%, and 34%, respectively. Also, Catalysts 15 and 16 demonstrate average acrylate STY of 307 g/hr/L and 335 g/hr/L, respectively, while Comp. B, Comp. C and Comp. D demonstrate average yields of only 287 g/hr/L, 254 g/hr/L and 338 g/hr/L, respectively. In addition, Catalysts 15 and 16 demonstrate average acrylate yields of 24% and 26%, respectively, while Comp. B, Comp. C, and Comp. C demonstrate an average yield of only 22%, 19%, and 26%, respectively.

As shown in Table 22, Catalysts 15 and 16 show steady acetic acid conversion, acrylate selectivity, acrylate yield, and acrylate STY. For example, Catalyst 15 has a 29-32% acetic acid conversion, 77-84% acrylate selectivity, 23-26% acrylate yield and 294-333 g/hr/L acrylate STY over a 21.8 hour period. Catalyst 16 has a 29-31% acetic acid conversion, 87-89% acrylate selectivity, 25-27% yield and 328-351 g/hr/L acrylate STY over a 21.8 hour period. The data shows that both catalysts have little deactivation over a long period of time. In comparison, acetic acid conversion, acrylate yield, and acrylate space time yield for Comp. B and Comp. D decreased after only 3.9 hours and 3.3 hours, respectively. Although Catalyst Comp. C shows a steady acetic acid conversion, acrylate selectivity, acrylate yield, and acrylate space time yield for a 4 hour period, the acetic acid conversion is at an undesirable 22%.

In addition, it appears that catalysts with a low level of bismuth and tungsten have similar or better acetic acid conversion and acrylate STY than commercially available VPO catalysts. For example, Catalysts 15 and 16 have an average acetic acid conversion of 30% and 29%, respectively and an average acrylate STY of 307 g/hr/L and 335 g/hr/L, respectively. In comparison, Comp. B, Comp. C, and Comp. D have an average acetic acid conversion of 24%, 22%, and 34%, respectively and an average STY of 287 g/hr/L, 254 g/hr/L, and 338 g/hr/L, respectively.

### Example 22

Catalyst compositions were prepared using a tungsten salt, a titanium salt, and a vanadium precursor, e.g., NH₄VO₃. An aqueous suspension of TiP₂O₇ was prepared by adding the finely powdered solid to 50 mL of deionized H₂O. Next, a calculated amount of phosphoric acid (85%) was added, the suspension heated to 80° C. with stirring and kept at this temperature for 30 min. Separately, an organic acid, e.g., oxalic acid or citric acid, was added was dissolved in deionized H₂O, and the solution was heated to about 50° C. with stirring. Next, calculated amounts of NH₄VO₃ and ammonium metatungstate hydrate were added in small portion over about 10 min, the solution was then heated to 80° C. with stirring, and kept at this temperature for 60 min. Next, the dark blue-green solution was then added to the suspension of TiP₂O₇ with stirring, and the final mixture was kept stirring for another 30 min at this temperature. The final mixture was then evaporated to dryness (rotary evaporator), and the resulting solid is dried (120° C.) overnight/air and calcined using the following temperature program:
(1) heating to 160° C. at a rate of 10° C. per minute, and holding at 160° C. for 2 hours;
(2) heating to 250° C. at a rate of 3° C. per minute, and holding at 250° C. for 2 hours;
(3) heating to 300° C. at a rate of 3° C. per minute, and holding at 300° C. for 6 hours; and
(4) heating to 450° C. at a rate of 3° C. per minute, and holding at 450° C. for 6 hours.

### Example 23

Colloidal silica and deionized water were combined and mixed. Ti(OiPr)₄ was mixed with iPrOH and this mixture was added to the water-colloidal silica mixture and stirred at room temperature for at least 30 minutes. Separately, an organic acid, e.g., oxalic acid or citric acid and ethylene glycol and water were combined, and was heated to 50° C. A calculated amount of NH₄VO₃ was added to the mixture and the resulting solution was heated to 80° C. with stirring. A calculated amount of ammonium metatungstate was added to the warm vanadium solution and was stirred for 15 minutes at 80° C. Optionally, a solution of methyl cellulose was added to the vanadium and tungsten mixture. The mixture was stirred at 80° C. for 15-30 minutes. The vanadium/tungsten mixture was cooled and was slowly added to the titania suspension/gel. The mixture was stirred for at least 15 minutes at room temperature. A calculated amount of phosphoric acid (85%) was added and the resulting solution was stirred. The final mixture was evaporated to dryness in a 120° C. drying oven overnight. The resulting solid was calcined using the temperature profile of Example 22.

The effect of vanadium, tungsten and/or titanium on the surface area, pore volume, and pore size of the resultant catalyst was studied. Catalysts 1 and 2 were prepared using citric acid as detailed in Example 2. Table 23 shows surface area, pore volume, and pore size of Catalysts 1 and 2. Catalyst 1 has a V:W:Ti ratio of 6.12:0.87:4 and Catalyst 2 has a V:W:Ti ratio of 1.75:0.25:4.

As shown in Table 23, Catalyst 1 has a 3.5 times more vanadium and tungsten than Catalyst 2.

TABLE 23
Catalyst Compositions

| Catalyst | Catalyst Formula | Preparation Details |
|---|---|---|
| 1 | V_{6.12}W_{0.87}Ti₄P₁₆O₆₂ | citric acid, ethylene glycol, 5.8% SiO₂ |
| 2 | V_{1.75}W_{0.25}Ti₄P₁₁O₄₀ | citric acid, ethylene glycol, 5.8% SiO₂ |

### Example 24

A reaction feed comprising acetic acid, formaldehyde, methanol, water, oxygen, and nitrogen was passed through a fixed bed reactor comprising Catalysts 1-2 shown in Table 23. The reactions for Catalysts 1-2 were conducted at a reactor temperature of 375° C. and a GHSV of 2000 Hr⁻¹, total organics of 32 mole %, acetic acid and formaldehyde ratio of 1.5, O₂ of 4.8%, H₂O of 7.2 mole %, total N₂ of 56 mole %. Acrylic acid and methyl acrylate (collectively, “acrylate product”) were produced. Acetic acid conversion, acrylate selectivity, acrylate yield, and acrylate space time yield were measured for Catalysts 1-3 a various time points of the reaction. Commercial VPO catalyst Comp. A and Comp. B were also tested under the same condition. The results are shown in Table 24.

TABLE 24
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 1 | 0.5 | 47 | 85 | 40 | 517 |
| | 1.5 | 46 | 85 | 39 | 509 |
| | 2.5 | 46 | 86 | 40 | 515 |
| | 3.1 | 45 | 86 | 39 | 506 |
| | 17.4 | 42 | 84 | 35 | 462 |
| | 19.4 | 42 | 84 | 35 | 456 |
| | 20.3 | 40 | 88 | 35 | 462 |
| 2 | 0.5 | 35 | 83 | 29 | 384 |
| | 1.5 | 35 | 83 | 29 | 377 |
| | 2.5 | 35 | 83 | 29 | 382 |
| | 3.1 | 35 | 82 | 28 | 371 |
| | 17.4 | 32 | 81 | 26 | 345 |
| | 18.4 | 33 | 81 | 26 | 345 |
| | 19.4 | 33 | 80 | 26 | 345 |
| | 20.3 | 33 | 81 | 27 | 350 |
| VPO | 0.8 | 27 | 85 | 23 | 304 |
| Comp. A | 1.7 | 24 | 94 | 22 | 289 |
| | 2.7 | 23 | 95 | 22 | 280 |
| | 3.9 | 22 | 97 | 21 | 277 |
| VPO | 0.8 | 22 | 90 | 20 | 255 |
| Comp. B | 1.7 | 22 | 90 | 19 | 253 |
| | 2.7 | 22 | 87 | 19 | 251 |
| | 3.9 | 22 | 90 | 19 | 254 |

Acetic acid conversion, acrylate selectivity, acrylate yield, and acrylate space time yield were measured for Catalysts 1 and 2 and Comp. A and Comp. B at various time points of the reaction. Comp. A is a commercially available VPO catalyst and Comp. B was prepared using citric acid, ethylene glycol, silica and 10% methyl cellulose. Catalysts 1 and 2, both of which contain vanadium, tungsten and titanium, unexpectedly outperform Comp. A and Comp. B, which are conventional tungsten-free and titanium-free commercially available vanadium catalysts. Catalysts 1 and 2 show better acetic acid conversions, acrylate yield, and acrylate STY than Comp. A and Comp. B. Specifically, Catalysts 1 and 2 show initial acetic acid conversion of 47% and 35%, respectively. In comparison, Comp. A and Comp. B show an initial acetic acid conversion of 27% and 22%, respectively.

Furthermore, surprisingly Catalysts 1 and 2 maintained steady acetic acid conversion, acrylate selectivity, acrylate yield, and acrylate space time yield over long time periods, i.e., Catalysts 1 and 2 showed little if any catalyst deactivation. For example, Catalyst 1 has an initial acetic acid conversion of 47% and reduces to 40% over the course of 20.3 hours; and Catalyst 2 has an initial acetic acid conversion of 35% and slightly reduces to 33% over 20.3 hours. In comparison, Comp. A has an initial acetic acid conversion of 27% and decreases to 22% over only 3.9 hours. Therefore, as shown by the data, Catalysts 1 and 2 outperform commercially available VPO catalyst.

### Example 25

Table 25 shows Catalysts 3-8, each of which are VWTiPO catalysts prepared via the preparation method of Example 23.

TABLE 25
Catalyst Compositions

| Catalyst | Catalyst Formula | Preparation Details |
|---|---|---|
| 3 | V₁₀W_{1.0}Ti₄P_{20.2}O₈₄ | oxalic acid, ethylene glycol, 8.3% SiO₂ |
| 4 | V₁₀W_{0.16}Ti₆P_{24.4}O₉₆ | oxalic acid, ethylene glycol, 8.0% SiO₂ |
| 5 | V₁₀W_{1.0}Ti₄P_{20.2}O₈₄ | oxalic acid, ethylene glycol, |
| | | 8.5% SiO₂, 10% MC |
| 6 | V₁₀W_{1.0}Ti₄P_{20.2}O₈₄ | citric acid, ethylene glycol, |
| | | 5.8% SiO₂, 10% MC |
| 7 | V₁₀W_{0.16}Ti₄P_{20.1}O₈₁ | citric acid, ethylene glycol, |
| | | 5.8% SiO₂, 10% MC |
| 8 | V₁₀W_{0.16}Ti₁₀P₃₃O₁₂₈ | citric acid, ethylene glycol, |
| | | 5.8% SiO₂, 10% MC |

Catalysts 3-5 were made using oxalic acid and between 8.0 to 8.5% SiO₂. Catalysts 6-8 were made using citric acid and 5.8% SiO₂. Catalysts 5-8 also included 10% methyl cellulose whereas catalysts 3 and 4 were free of methyl cellulose. Catalysts 3, 5, and 6 have V:W:Ti ratio of 10:1.0:4. Catalyst 4 has a V:W:Ti ratio of 10:0.16:6. Catalyst 7 has a V:W:Ti ratio of 10:0.16:4. Catalyst 8 has a V:W:Ti ratio of 10:0.16:10.

### Example 26

A reaction feed comprising acetic acid, formaldehyde, methanol, water, oxygen, and nitrogen was passed through a fixed bed reactor comprising Catalysts 3-8 shown in Table 25. The reactions for Catalysts 3-8 were conducted at a reactor temperature of 375° C. and a GHSV of 2000 Hr⁻¹, total organics of 32 mole %, acetic acid and formaldehyde ratio of 1.5, O₂ of 4.8%, H₂O of 7.2 mole %, total N₂ of 56 mole %, and formalin was fed as trioxane. Acrylic acid and methyl acrylate (collectively, “acrylate product”) were produced. Acetic acid conversion, acrylate selectivity, acrylate yield, and acrylate space time yield were measured for Catalysts 3-8 at various time points of the reaction. A commercial VPO catalyst Comp. A was also tested under the same condition. The results are shown in Table 26.

TABLE 26
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 3 | 0.5 | 27 | 81 | 22 | 287 |
| | 1.4 | 28 | 81 | 22 | 296 |
| | 2.0 | 28 | 82 | 23 | 303 |
| | 16.3 | 28 | 81 | 23 | 303 |
| | 17.3 | 28 | 81 | 23 | 305 |
| | 18.0 | 28 | 81 | 23 | 304 |
| | Avg. | 28 | 81 | 23 | 300 |
| 4 | 0.5 | 30 | 88 | 26 | 348 |
| | 1.4 | 32 | 89 | 28 | 369 |
| | 2.0 | 32 | 89 | 28 | 373 |
| | 16.3 | 34 | 87 | 29 | 387 |
| | 17.3 | 34 | 88 | 30 | 396 |
| | 18.0 | 34 | 88 | 30 | 393 |
| | Avg. | 33 | 88 | 29 | 378 |
| 5 | 0.5 | 35 | 90 | 31 | 409 |
| | 1.5 | 35 | 90 | 31 | 409 |
| | 2.7 | 35 | 90 | 31 | 410 |
| | 3.8 | 35 | 89 | 31 | 408 |
| | 22.4 | 36 | 90 | 32 | 422 |
| | 23.6 | 35 | 93 | 33 | 430 |
| | 24.7 | 35 | 95 | 33 | 435 |
| | 25.7 | 37 | 87 | 32 | 421 |
| | Avg. | 35 | 90 | 32 | 418 |
| 6 | 0.5 | 37 | 84 | 31 | 406 |
| | 1.5 | 35 | 84 | 30 | 394 |
| | 2.3 | 35 | 84 | 29 | 388 |
| | 17.1 | 36 | 83 | 30 | 391 |
| | 18.3 | 35 | 84 | 30 | 392 |
| | 19.3 | 35 | 83 | 29 | 389 |
| | Avg. | 36 | 84 | 30 | 393 |
| 7 | 2.3 | 30 | 82 | 24 | 323 |
| | 3.5 | 30 | 83 | 25 | 328 |
| | 17.1 | 30 | 82 | 24 | 323 |
| | 18.4 | 29 | 83 | 25 | 326 |
| | 19.6 | 29 | 82 | 24 | 320 |
| | Avg. | 30 | 83 | 24 | 324 |
| 8 | 0.7 | 26 | 86 | 22 | 297 |
| | 1.6 | 27 | 85 | 23 | 300 |
| | 2.4 | 27 | 86 | 24 | 311 |
| | 17.5 | 30 | 83 | 25 | 336 |
| | 18.6 | 31 | 85 | 26 | 345 |
| | 19.8 | 31 | 83 | 26 | 340 |
| | Avg. | 29 | 85 | 24 | 321 |
| VPO | 0.8 | 27 | 85 | 23 | 304 |
| Comp. A | 1.7 | 24 | 94 | 22 | 289 |
| | 2.7 | 23 | 95 | 22 | 280 |
| | 3.9 | 22 | 97 | 21 | 277 |
| | Avg. | 24 | 93 | 22 | 287 |

Catalysts 3-8, all of which contain vanadium, tungsten and titanium, unexpectedly outperform Comp. A, which is conventional tungsten-free and titanium-free commercially available catalysts. For example, Catalysts 3-8 demonstrate average acetic acid conversions of 28%, 33%, 35%, 36%, 30%, and 39%, respectively, while Comp. A demonstrates an average acetic acid conversion of only 24%. Also, Catalysts 3-8 demonstrate average acrylate STY of 300 g/hr/L, 378 g/hr/L, 418 g/hr/L, 393 g/hr/L, 324 g/hr/L, and 321 g/hr/L, respectively, while Comp. A demonstrates an average STY of only 287 g/hr/L. In addition, Catalysts 3-8 demonstrate average acrylate yields of 23%, 29%, 32%, 30%, 24% and 24%, respectively, while Comp. A demonstrates an average yield of only 22%.

Surprisingly and unexpectedly, as shown, all catalysts maintained steady or increase acetic acid conversion, acrylate selectivity, acrylate yield, and acrylate space time yield over long time periods, i.e., Catalysts 3-8 showed little if any catalyst deactivation. For example, over a 18.0 hour period, the acetic acid conversion for Catalyst 3 remains between 27% and 28%. Over a 18.0 hour period, the acetic acid conversion for Catalyst 4 increases from 30% to 34%. Similarly, over a 25.7 hour period, the acetic acid conversion for Catalyst 5 remains between 35% and 37%. In addition, over a 18.0 hour period, the acetic acid conversion for Catalyst 6 remains between 35% to 37%. Similarly, over a 19.6 hour period, the acetic acid conversion for Catalyst 7 remains between 29% to 30%. Over a 19.8 hour period, the acetic acid conversion for Catalyst 8 increases from 26% to 31%.

In comparison, acetic acid conversion for Comp. A decreased significantly from 27% to 22% within only 3.9 hours

As shown in Table 26, Catalysts 3-8 have steady acrylate yield and acrylate space time yield. For example, Catalyst 3 has an acrylate yield between 22% to 23% and acrylate space time yield between 287 g/hr/L and 305 g/hr/L. Catalyst 4 has an acrylate yield between 26% to 30% and acrylate space time yield increase from 348 g/hr/L to 396 g/hr/L. Catalyst 5 has an acrylate yield between 31% to 33% and acrylate space time yield between 408 g/hr/L and 435 g/hr/L. Catalyst 6 has an acrylate yield between 29% to 31% and acrylate space time yield between 388 g/hr/L and 406 g/hr/L. Catalyst 7 has an acrylate yield between 24% to 25% and acrylate space time yield between 320 g/hr/L and 328 g/hr/L. Catalyst 8 has an acrylate yield between 22% to 26% and acrylate space time yield between 297 g/hr/L and 345 g/hr/L.

Therefore, as shown by the data, Catalysts 3-8, which comprise vanadium, tungsten, and titanium outperform commercially available VPO catalyst.

### Example 27

Table 27 shows Catalysts 9-12, each of which are VWTiPO catalysts prepared via the preparation method of Example 23.

TABLE 27
Catalyst Compositions

| Catalyst | Catalyst Formula | Preparation Details |
|---|---|---|
| 9 | V₁₀W_{1.0}Ti_{0.16}P_{11.9}O₅₅ | oxalic acid, ethylene glycol, 8.2% SiO₂ |
| 10 | V₁₀W_{1.0}Ti_{0.16}P_{11.9}O₅₅ | oxalic acid, ethylene glycol, |
| | | 8.6% SiO₂, 10% MC |
| 11 | V₁₀W_{1.0}Ti_{0.16}P_{11.9}O₅₅ | citric acid, ethylene glycol, |
| | | 5.8% SiO₂, 10% MC |
| 12 | V₁₀W_{0.16}Ti_{0.16}P_{11.6}O₅₂ | citric acid, ethylene glycol, |
| | | 5.8% SiO₂, 10% MC |

Catalysts 9 and 10 were made using oxalic acid, ethylene glycol, and 8.2% and 8.5% SiO₂, respectively. Catalysts 11 and 12 were made using citric acid, ethylene glycol, and 5.8% SiO₂. Catalysts 10-12 also included 10% methyl cellulose whereas Catalyst 9 was free of methyl cellulose. Catalysts 9, 10, and 11 have V:W:Ti ratio of 10:1.0:0.16. Catalyst 12 has a V:W:Ti ratio of 10:0.16:0.16.

### Example 28

A reaction feed comprising acetic acid, formaldehyde, methanol, water, oxygen, and nitrogen was passed through a fixed bed reactor comprising Catalysts 9-12 shown in Table 27. The reactions for Catalysts 9-12 were conducted at a reactor temperature of 375° C. and a GHSV of 2000 Hr⁻¹, total organics of 32 mole %, acetic acid and formaldehyde ratio of 1.5, O₂ of 4.8%, H₂O of 7.2 mole %, total N₂ of 56 mole %, and formalin was fed as trioxane. Acrylic acid and methyl acrylate (collectively, “acrylate product”) were produced. Acetic acid conversion, acrylate selectivity, acrylate yield, and acrylate space time yield were measured for Catalysts 9-12 at various time points of the reaction. A commercial VPO catalyst Comp. A was also tested under the same condition. The results are shown in Table 28.

TABLE 28
Acrylate Production

| | | HOAc | | | Acrylate |
|---|---|---|---|---|---|
| | Runtime | Conv. | Acrylate | Acrylate | STY |
| Catalyst | (h) | (%) | Selectivity (%) | Yield (%) | (g/hr/L) |
| 9 | 0.5 | 20 | 88 | 18 | 234 |
| | 1.5 | 21 | 88 | 18 | 238 |
| | 2.3 | 21 | 87 | 18 | 240 |
| | 17.1 | 21 | 86 | 18 | 239 |
| | 18.3 | 21 | 87 | 19 | 244 |
| | 19.3 | 21 | 87 | 18 | 241 |
| | Avg. | 21 | 87 | 18 | 239 |
| 10 | 0.5 | 33 | 87 | 29 | 383 |
| | 1.5 | 33 | 87 | 29 | 386 |
| | 2.7 | 34 | 87 | 29 | 388 |
| | 3.8 | 34 | 87 | 29 | 390 |
| | 22.4 | 34 | 85 | 29 | 387 |
| | 23.6 | 34 | 86 | 29 | 389 |
| | 24.7 | 35 | 85 | 29 | 390 |
| | 25.7 | 34 | 85 | 29 | 387 |
| | Avg. | 34 | 86 | 29 | 388 |
| 11 | 2.3 | 44 | 80 | 35 | 454 |
| | 3.5 | 44 | 81 | 35 | 455 |
| | 17.1 | 43 | 80 | 34 | 442 |
| | 18.4 | 43 | 81 | 35 | 451 |
| | 19.6 | 43 | 81 | 35 | 450 |
| | Avg. | 43 | 81 | 35 | 450 |
| 12 | 0.7 | 28 | 86 | 24 | 318 |
| | 1.6 | 28 | 87 | 25 | 324 |
| | 2.4 | 28 | 87 | 25 | 325 |
| | 17.5 | 28 | 85 | 24 | 312 |
| | 18.6 | 28 | 86 | 24 | 314 |
| | 19.8 | 28 | 86 | 24 | 313 |
| | Avg. | 28 | 86 | 24 | 318 |
| VPO | 0.8 | 27 | 85 | 23 | 304 |
| Comp. A | 1.7 | 24 | 94 | 22 | 289 |
| | 2.7 | 23 | 95 | 22 | 280 |
| | 3.9 | 22 | 97 | 21 | 277 |
| | Avg. | 24 | 93 | 22 | 287 |

Catalysts 9-12, all of which contain vanadium, tungsten and titanium, unexpectedly outperform Comp. A, which is conventional tungsten-free and titanium-free VPO catalysts. For example, Catalysts 10-13 demonstrate average acetic acid conversions of 21%, 34%, 43%, and 28%, respectively, while Comp. A demonstrates average acetic acid conversion of only 24%. Also, Catalysts 9-12 demonstrate average acrylate STY of 239 g/hr/L, 388 g/hr/L, 450 g/hr/L, and 318 g/hr/L, respectively, while Comp. A demonstrates an average yield of only 287 g/hr/L. In addition, Catalysts 9-12 demonstrate average acrylate yields of 18%, 29%, 33%, and 24%, respectively, while Comp. A demonstrates average yield of only 22%.

Surprisingly and unexpectedly, as shown, all catalysts maintained steady or increase acetic acid conversion, acrylate selectivity, acrylate yield, and acrylate space time yield over long time periods, i.e., Catalysts 9-12 showed little if any catalyst deactivation. For example, over a 19.3 hour period, the acetic acid conversion for Catalyst 9 remains between 20% and 21%. Over a 25.7 hour period, the acetic acid conversion for Catalyst 10 remains between 33% and 34%. Similarly, over a 19.6 hour period, the acetic acid conversion for Catalyst 11 remains between 44% and 43%. In addition, over a 19.8 hour period, the acetic acid conversion for Catalyst 12 remains at 28%. In comparison, acetic acid conversion of Comp. A decreases significantly from 27% to 22% within only 3.9 hours.

It appears that catalysts with a low level of titanium have similar or better acetic acid conversion and acrylate STY than commercially available VPO catalysts. For example, Catalysts 9-11 have steady acrylate yield and acrylate space time yield. For example, Catalyst 9 has a steady acrylate yield of 18% and acrylate space time yield between 234 g/hr/L and 244 g/hr/L. Catalyst 10 has a steady acrylate yield of 29% and acrylate space time yield between 383 g/hr/L and 390 g/hr/L. Catalyst 11 has an acrylate yield between 34% to 35% and acrylate space time yield between 442 g/hr/L and 455 g/hr/L.

Surprisingly and unexpectedly, it appears that catalyst with low level of titanium and tungsten has similar or better acetic acid conversion and acrylate STY than commercially available VPO catalysts. For example, Catalyst 12 has a steady acrylate yield between 24% and 25% and acrylate space time yield between 312 g/hr/L and 325 g/hr/L.

Therefore, as shown by the data, Catalysts 9-12, which comprise vanadium, tungsten, and titanium outperform commercially available VPO catalyst.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing an acrylate product, the process comprising the steps of:
(a) reacting, over at least one oxidation catalyst, in a first reaction zone, a reaction gas mixture A comprising methanol, oxygen, and at least one diluent gas other than steam to form a product gas mixture A comprising formaldehyde, steam, and at least one inert diluent gas other than steam;
(b) combining at least a portion of the product gas mixture A and acetic acid to form a reaction gas mixture B comprising acetic acid, formaldehyde, steam, and at least one diluent gas other than steam to form a product gas comprising formaldehyde, steam, and at least one inert diluent gas other than steam;
(c) reacting, over at least one aldol condensation catalyst, in a second reaction zone, at least a portion of the acetic acid in the reaction gas input mixture B with at least a portion of the formaldehyde in the reaction gas input mixture B to form a product gas mixture B comprising acrylic acid, acetic acid, steam, and at least one inert diluent gas other than steam; and
wherein the at least one aldol condensation catalyst comprises an active phase comprising vanadium and bismuth.

2. The process of claim 1, wherein the molar ratio of vanadium to bismuth in the active phase of the at least one aldol condensation catalyst composition is at least 0.02:1.

3. The process of claim 1, wherein the active phase of the aldol condensation catalyst comprises from 0.3 wt. % to 32 wt. % vanadium; and/or from 0.1 wt. % to 75 wt. % bismuth.

4. The process of claim 1, wherein the aldol condensation catalyst corresponds to the formula
VₐBi_{b}P_{c}O_{d},
wherein:
a is 1 to 100,
b is from 0.1 to 50,
c is from 1 to 165, and
d is from 4 to 670.

5. The process of claim 1, wherein the active phase further comprises titanium.

6. The process of claim 5, wherein the active phase of the aldol condensation catalyst comprises from 0.015 wt. % to 22 wt. % titanium.

7. The process of claim 5, wherein the catalyst corresponds to the formula
VₐBi_{b}Ti_{c}P_{d}Oₑ,
wherein:
a is 1 to 100,
b is from 0.1 to 50,
c is from 0.1 to 50,
d is from 1.5 to 270,
e is from 6 to 1045.

8. The process of claim 1, wherein the active phase further comprises tungsten.

9. The process of claim 8, wherein the active phase comprises from 0.1 wt. % to 61 wt. % tungsten.

10. The process of claim 8, wherein a molar ratio of vanadium to tungsten in the active phase of the catalyst composition is at least 0.033:1, and wherein a molar ratio of bismuth to tungsten in the active phase of the catalyst composition is at least 0.0033:1.

11. The process of claim 8, wherein a molar ratio of vanadium to bismuth in the active phase of the catalyst composition is at least 0.033:1, and wherein a molar ratio of vanadium to tungsten in the active phase of the catalyst composition is at least 0.033:1.

12. The process of claim 8, wherein the catalyst corresponds to the formula
VₐBi_{b}W_{c}P_{d}Oₑ,
wherein
a is from 1 to 100,
b is from 0.1 to 30,
c is from 0.1 to 30,
d is from 1.0 to 175, and
e is from 5 to 710.

13. The process of claim 1, wherein the at least one oxidation catalyst comprises a catalytically active material which is a mixed oxide of the general formula I
[Fe₂(MoO₄)₃]₁[M¹ₘOₙ]_{q} (I)
in which the variables are each defined as follows:
M¹ is
Mo and/or Fe or
Mo and/or Fe and, based on the total molar amount of Mo and Fe, a total molar amount of up to 10 mol % of one or more elements from the group consisting of Ti, Sb, Sn, Ni, Cr, Ce, Al, Ca, Mg, V, Nb, Ag, Mn, Cu, Co, Si, Na, K, Tl, Zr, W, Ir, Ta, As, P and B,
q is 0 to 5,
m is 1 to 3,
n is 1 to 6.

14. A process for producing an acrylate product, the process comprising the steps of:
(a) reacting, over at least one oxidation catalyst, in a first reaction zone, a reaction gas mixture A comprising methanol, oxygen, and at least one diluent gas other than steam to form a product gas mixture A comprising formaldehyde, steam, and at least one inert diluent gas other than steam;
(b) combining at least a portion of the product gas mixture A and acetic acid to form a reaction gas mixture B comprising acetic acid, formaldehyde, steam, and at least one diluent gas other than steam to form a product gas comprising formaldehyde, steam, and at least one inert diluent gas other than steam;
(c) reacting, over at least one aldol condensation catalyst, in a second reaction zone, at least a portion of the acetic acid in the reaction gas input mixture B with at least a portion of the formaldehyde in the reaction gas input mixture B to form a product gas mixture B comprising acrylic acid, acetic acid, steam, and at least one inert diluent gas other than steam; and
wherein the at least one aldol condensation catalyst comprises an active phase comprising vanadium and one or more of titanium and tungsten.

15. The process of claim 14 wherein the at least one aldol condensation catalyst comprises an active phase comprising vanadium, titanium and tungsten and wherein a molar ratio of vanadium to tungsten in the active phase of the catalyst composition is at least 0.02:1.

16. The process of claim 15, wherein the active phase comprises from 0.2 wt. % to 30 wt. % vanadium; and/or from 0.016 wt. % to 20 wt. % titanium; and/or from 0.11 wt. % to 65 wt. % tungsten.

17. The process of claim 14, wherein the catalyst corresponds to the formula
VₐTi_{b}W_{c}P_{d}Oₑ,
wherein:
a is from 1 to 100,
b is from 0.1 to 50,
c is from 0.1 to 50,
d is from 1 to 270,
e is from 6 to 1040.

18. The process of claim 14 wherein the at least one aldol condensation catalyst comprises an active phase comprising vanadium and titanium and wherein a molar ratio of vanadium to titanium in an active phase of the catalyst composition is greater than 0.5:1.

19. The process of claim 14, wherein the at least one aldol condensation catalyst comprises vanadium and titanium;
wherein the at least one aldol condensation catalyst further comprises at least one oxide additive in an amount of at least 0.1 wt % based on the total weight of the aldol condensation catalyst; and
wherein the molar ratio of oxide additive to titanium in an active phase of the at least one aldol condensation catalyst is at least 0.05:1.

20. The process of claim 14, wherein the at least one aldol condensation catalyst corresponds to the formula
VₐTi_{b}P_{c}O_{d}(oxide additive)ₑ
wherein
a is from 1 to 8;
b is from 4 to 8;
c is from 10 to 30
d is from 30 to 70 and
e is from 0.01 to 500.
